SEQUENCE LISTING (140 sequences)

SEQ ID NO: 1 - protein, 343 aa, unidentified
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 2 - protein, 343 aa, synthetic construct
  REGION 1..343: note = mutated protein
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTRTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 3 - protein, 343 aa, synthetic construct
  REGION 1..343: note = mutated protein
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQEYLAWSGHSARVGAARDMARAGVSIPEIMQAGGWTNVNIVMNYIRNLDSETGAMVRLLEDGD

SEQ ID NO: 4 - protein, 343 aa, synthetic construct
  REGION 1..343: note = mutated protein
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMKAGGWTNVNIVMNYIRNLDSETGAMVRLLEDGD

SEQ ID NO: 5 - protein, 343 aa, synthetic construct
  REGION 1..343: note = mutated protein
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMRAGGWTNVNIVMNYIRNLDSETGAMVRLLEDGD

SEQ ID NO: 6 - protein, 343 aa, synthetic construct
  REGION 1..343: note = recombinant protein
MSNLQTLHQNLSALLANATSDEARKNLMDVFRDRRAFSEATWKTLLSVCRTWAAWCKLNNRKWFPAEPEDVRDYLLHLQVRGLAVNTIQRHLALLNMLHRRSGLPRPGDSSAVSLVMRRIRKENVDAGERVRQALAFERTDFDKVRSLMGNSDRCQDIRNLAFLGVAYNTLLRISEIARIRIKDISRTDGGRMLIHIGRTKTLVSTAGVEKALSLGVTRLVGRWISVSGVAGDPNNYLFCRVRKNGVAAPSATSQLSTDVLRGVFAAAHRLVYGTKDDSGQGYLTWSGHSARVGAARDMARAGVSIAEIMQAGGWTTVESVMSYLRNLDSETGAMVRLLEDGD

SEQ ID NO: 7 - protein, 343 aa, synthetic construct
  REGION 1..343: note = mutated protein
MSNLQTLHQNLSALLANATSDEARKNLMDVFRDRRAFSEATWKTLLSVCRTWAAWCKLNNRKWFPAEPEDVRDYLLHLQVRGLAVNTIQRHLALLNMLHRRSGLPRPGDSSAVSLVMRRIRKENVDAGERVRQALAFERTDFDKVRSLMGNSDRCQDIRNLAFLGVAYNTLLRISEIARIRIKDISRTDGGRMLIHIGRTRTLVSTAGVEKALSLGVTRLVGRWISVSGVAGDPNNYLFCRVRKNGVAAPSATSQLSTDVLRGVFAAAHRLVYGTKDDSGQGYLTWSGHSARVGAARDMARAGVSIAEIMQAGGWTTVESVMSYLRNLDSETGAMVRLLEDGD

SEQ ID NO: 8 - protein, 343 aa, synthetic construct
  REGION 1..343: note = mutated protein
MSNLQTLHQNLSALLANATSDEARKNLMDVFRDRRAFSEATWKTLLSVCRTWAAWCKLNNRKWFPAEPEDVRDYLLHLQVRGLAVNTIQRHLALLNMLHRRSGLPRPGDSSAVSLVMRRIRKENVDAGERVRQALAFERTDFDKVRSLMGNSDRCQDIRNLAFLGVAYNTLLRISEIARIRIKDISRTDGGRMLIHIGRTKTLVSTAGVEKALSLGVTRLVGRWISVSGVAGDPNNYLFCRVRKNGVAAPSATSQLSTDVLRGVFAAAHRLVYGTKDDSGQEYLTWSGHSARVGAARDMARAGVSIAEIMQAGGWTTVESVMSYLRNLDSETGAMVRLLEDGD

SEQ ID NO: 9 - protein, 343 aa, synthetic construct
  REGION 1..343: note = rceombinant protein
MSNLQTLHQNLSALLANATSDEARRNLMRVFELRRAFSEATWKTLLSVCRTWAAWCKLNNRKWFPAEPEDVRDYLLHLQVRGLAVNTIQRHLALLNMLHRRSGLPRPGDSSAVSLVMRRIRKLNVDAGERVRQALAFERTDFDKVRSLMGNSDRCQDIRNLAFLGVAYNTLLRISEIARIRIKDISRTDGGRMLIHIGRTKTLVSTAGVEKALSLGVTRLVGRWISVSGVAGDPNNYLFCRVRKNGVAAPSATSQLSTDVLRGVFAAAHRLVYGTKDDSGQGYLTWSGHSARVGAARDMARAGVSIAEIMQAGGWTTVESVMSYLRNLDSETGAMVELLEDGD

SEQ ID NO: 10 - protein, 343 aa, synthetic construct
  REGION 1..343: note = rcemombinant protein
MSNIQTPHQSLSALLTDATSDVTRKNLADMFRDSQAFSEHTWKMLLSVCRSWAAWCELNNRKWLPVEPEDVRDYLLHLQTRGLAVKTIQHHLGSLNMLHRRAGLPRPGDSNAVSLVMRRIRRENVDAGERAQQALAFERTDFDQVRSLVENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFGAAHRLVYGAKGDSGQKYLAWSGHSARVGAARDMARAGVPIPEIMQAGGWTTVNSVMNYIRNLDSETGAMVRLLEDSD

SEQ ID NO: 11 - protein, 343 aa, synthetic construct
  REGION 1..343: note = mutated protein
MSNIQTPHQSLSALLTDATSDVTRKNLADMFRDSQAFSEHTWKMLLSVCRSWAAWCELNNRKWLPVEPEDVRDYLLHLQTRGLAVKTIQHHLGSLNMLHRRAGLPRPGDSNAVSLVMRRIRRENVDAGERAQQALAFERTDFDQVRSLVENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFGAAHRLVYGAKGDSGQKYLAWSGHSARVGAARDMARAGVPIPEIMRAGGWTTVNSVMNYIRNLDSETGAMVRLLEDSD

SEQ ID NO: 12 - protein, 343 aa, synthetic construct
  REGION 1..343: note = mutated protein
MSNIQTPHQSLSALLTDATSDVTRKNLADMFRDSQAFSEHTWKMLLSVCRSWAAWCELNNRKWLPVEPEDVRDYLLHLQTRGLAVKTIQHHLGSLNMLHRRAGLPRPGDSNAVSLVMRRIRRENVDAGERAQQALAFERTDFDQVRSLVENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFGAAHRLVYGAKGDSGQKYLAWSGHSARVGAARDMARAGVPIPEIMKAGGWTTVNSVMNYIRNLDSETGAMVRLLEDSD

SEQ ID NO: 13 - protein, 343 aa, synthetic construct
  REGION 1..343: note = recombinant protein
MSNIQTPHQSLSALLTDATSDVTRKNLADMFRDSQAFSEHTWKMLLSVCRSWAAWCELNNRKWLPVEPDDVKDYLEHLQTRGLAVKTIQHHLGSLNMLHRRAGLPRPGDSNAVSLVMRRIRRENVDAGERAQQALAFERTDFDQVRSLVENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFGAAHRLVYGAKGDSGQKYLAWSGHSARVGAARDMARAGVPIPRIMQAGGWTTVNSVMNYIRNLDSETGAMVRLLEDSD

SEQ ID NO: 14 - protein, 361 aa, unidentified
MTDLTPFPPLEHLEPDEFADLVRKAIKRDPQAGAHPAIQSAISHFQDEFVRRQGEWQPATLQRLRNAWNVFVRWCTHQGIPALPARHQDVERYLIERRNELHRNTLKVHLWAIGKTHVISGLPNPCAHRYVKAQMAQITHQKVRERERIEQAPAFRESDLDRLTELWSATRSVTQQRDLMIVSLAYETLLRKNNLEQMKVGDIEFCQDGSALITIPFSKTNHSGRDDVRWISPQVANQVHAYLQLPNIDADPQCFLLQRVKRSGKALNPESHNTLNGHHPVSEKLISRVFERAWRALNHETGPRYTGHSARVGAAQDLLQEGYSTLQVMQAGGWSSEKMVLRYGRHLHAHTSAMAQKRRQR

SEQ ID NO: 15 - protein, 361 aa, synthetic construct
  REGION 1..361: note = mutated protein
MTDLTPFPPLEHLEPDEFADLVRKAIKRDPQAGAHPAIQSAISHFQDEFVRRQGEWQPATLQRLRNAWNVFVRWCTHQGIPALPARHQDVERYLIERRNELHRNTLKVHLWAIGKTHVISGLPNPCAHRYVKAQMAQITHQKVRERERIEQAPAFRESDLDRLTELWSATRSVTQQRDLMIVSLAYETLLRKNNLEQMKVGDIEFCQDGSALITIPFSRTNHSGRDDVRWISPQVANQVHAYLQLPNIDADPQCFLLQRVKRSGKALNPESHNTLNGHHPVSEKLISRVFERAWRALNHETGPRYTGHSARVGAAQDLLQEGYSTLQVMQAGGWSSEKMVLRYGRHLHAHTSAMAQKRRQR

SEQ ID NO: 16 - protein, 361 aa, synthetic construct
  REGION 1..361: note = mutated protein
MTDLTPFPPLEHLEPDEFADLVRKAIKRDPQAGAHPAIQSAISHFQDEFVRRQGEWQPATLQRLRNAWNVFVRWCTHQGIPALPARHQDVERYLIERRNELHRNTLKVHLWAIGKTHVISGLPNPCAHRYVKAQMAQITHQKVRERERIEQAPAFRESDLDRLTELWSATRSVTQQRDLMIVSLAYETLLRKNNLEQMKVGDIEFCQDGSALITIPFSKTNHSGRDDVRWISPQVANQVHAYLQLPNIDADPQCFLLQRVKRSGKALNPESHNTLNGHHPVSEKLISRVFERAWRALNHETGPRYTGHSARVGAAQDLLQEGYSTLQVMRAGGWSSEKMVLRYGRHLHAHTSAMAQKRRQR

SEQ ID NO: 17 - protein, 343 aa, unidentified
MKHLAQIKTGALPAELTQLTPEDIANNLRRFIADKAAYSENTFRDLLSVIRRWAFWCNERDVGYLPIDPELAREYFLQMAESGLASSTIDKHYAMMNMLCRESGLPDLRGSVDLKRSMKRIRREAVLQGERTGQAVPFRLPDLQLLSHLMGRSDRLTDQRNLAFLFVAYNTLCRMSELSRIRVRDLDISDSGHVVINLSHTKTMVTAAGVIKHLSRAAAGHLMHWLELSGLIHHPDAMVFGPVRHNNTAGVSEKPMSAPATEKIFKDAWDLLGKEPVQDNKGRYAKWSGHSARVGAAMDMAERDATITQIMQEGTWQDPKTVMRYLRRSESQKGKMSGILDGE

SEQ ID NO: 18 - protein, 343 aa, synthetic construct
  REGION 1..343: note = mutated protein
MKHLAQIKTGALPAELTQLTPEDIANNLRRFIADKAAYSENTFRDLLSVIRRWAFWCNERDVGYLPIDPELAREYFLQMAESGLASSTIDKHYAMMNMLCRESGLPDLRGSVDLKRSMKRIRREAVLQGERTGQAVPFRLPDLQLLSHLMGRSDRLTDQRNLAFLFVAYNTLCRMSELSRIRVRDLDISDSGHVVINLSHTRTMVTAAGVIKHLSRAAAGHLMHWLELSGLIHHPDAMVFGPVRHNNTAGVSEKPMSAPATEKIFKDAWDLLGKEPVQDNKGRYAKWSGHSARVGAAMDMAERDATITQIMQEGTWQDPKTVMRYLRRSESQKGKMSGILDGE

SEQ ID NO: 19 - protein, 343 aa, synthetic construct
  REGION 1..343: note = mutated protein
MKHLAQIKTGALPAELTQLTPEDIANNLRRFIADKAAYSENTFRDLLSVIRRWAFWCNERDVGYLPIDPELAREYFLQMAESGLASSTIDKHYAMMNMLCRESGLPDLRGSVDLKRSMKRIRREAVLQGERTGQAVPFRLPDLQLLSHLMGRSDRLTDQRNLAFLFVAYNTLCRMSELSRIRVRDLDISDSGHVVINLSHTKTMVTAAGVIKHLSRAAAGHLMHWLELSGLIHHPDAMVFGPVRHNNTAGVSEKPMSAPATEKIFKDAWDLLGKEPVQDNKGRYAKWSGHSARVGAAMDMAERDATITQIMREGTWQDPKTVMRYLRRSESQKGKMSGILDGE

SEQ ID NO: 20 - protein, 342 aa, unidentified
MSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLSRSERLVDLRNRAFLFVAYNTLMRMSEISRIRVGDLDQTGDTVTLHISHTKTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMQEGTWKKPETLMRYLRRGGVSVGANSRLMDS

SEQ ID NO: 21 - protein, 342 aa, synthetic construct
  REGION 1..342: note = mutated protein
MSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLSRSERLVDLRNRAFLFVAYNTLMRMSEISRIRVGDLDQTGDTVTLHISHTRTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMQEGTWKKPETLMRYLRRGGVSVGANSRLMDS

SEQ ID NO: 22 - protein, 342 aa, synthetic construct
  REGION 1..342: note = mutated protein
MSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLSRSERLVDLRNRAFLFVAYNTLMRMSEISRIRVGDLDQTGDTVTLHISHTKTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMREGTWKKPETLMRYLRRGGVSVGANSRLMDS

SEQ ID NO: 23 - protein, 380 aa, unidentified
MIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCIKPKKDKQTGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKD

SEQ ID NO: 24 - protein, 380 aa, synthetic construct
  REGION 1..380: note = mutated protein
MIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITRTNHSGEPDTCILSQDVVSLLMDYTEAGKLDMSSDGFLFVGVSKHNTCIKPKKDKQTGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKD

SEQ ID NO: 25 - protein, 380 aa, synthetic construct
  REGION 1..380: note = mutated protein
MIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCIKPKKDKQTGEVLHKPITTKTVEGVFYSAWETLDLGRQGVKPFTAHSARVGAAQDLLKKGYNTLQIQRSGRWSSGAMVARYGRAILARDGAMAHSRVKTRSAPMQWGKDEKD

SEQ ID NO: 26 - DNA, 1032 bp, unidentified
  source 1..1032: mol_type = other DNA
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 27 - DNA, 1032 bp, synthetic construct
  misc_feature 1..1032: note = mutated sequence
  source 1..1032: mol_type = other DNA
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgcgcacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 28 - DNA, 1032 bp, synthetic construct
  misc_feature 1..1032: note = mutated sequence
  source 1..1032: mol_type = other DNA
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcaggaatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 29 - DNA, 1032 bp, synthetic construct
  misc_feature 1..1032: note = mutated sequence
  source 1..1032: mol_type = other DNA
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgaaagctggtggctggaccaatgtaaatattgtcatgaactatatccgtaacctggatagtgaaacaggggcaatggtgcgcctgctggaagatggcgattag

SEQ ID NO: 30 - DNA, 1032 bp, synthetic construct
  misc_feature 1..1032: note = muttaed sequence
  source 1..1032: mol_type = other DNA
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcgcgctggtggctggaccaatgtaaatattgtcatgaactatatccgtaacctggatagtgaaacaggggcaatggtgcgcctgctggaagatggcgattag

SEQ ID NO: 31 - DNA, 1032 bp, synthetic construct
  misc_feature 1..1032: note = recombinant sequence
  source 1..1032: mol_type = other DNA
atgtccaatctacagaccctacaccagaatttgtccgcactactggccaatgcaacaagtgatgaggctcgcaagaacctgatggacgtcttcagggaccgccgggcgttttctgaagctacctggaaaacgcttctgtccgtttgccggacgtgggcggcatggtgcaagctgaataaccggaaatggtttcccgcagaacctgaagatgtacgcgattatcttctacatctccaggtacgcggtctggcggtgaacacaatccagcgacacttggccctgctaaacatgcttcaccgtcggtccgggctgccgcgaccaggtgacagcagcgctgtttcactggttatgcggcgtatccgaaaggaaaacgttgatgccggtgaacgcgtaaggcaggccctggcgttcgagcgcactgatttcgacaaggttcgttcactcatgggaaacagcgatcgctgccaggatatacgtaatctggcatttctgggtgttgcatataacaccctgctacgcatatccgaaattgccaggattcggataaaagatatctcacgtaccgacggtgggaggatgctaatccatatcggcagaacgaaaacgctggttagcaccgcaggcgtagagaaggcacttagccttggggtaaccagactggtcgggcggtggatttctgtctctggtgtagctggcgacccgaataactacctgttctgccgggtcagaaaaaatggtgttgccgcgccatctgccaccagccagctatcgaccgacgtcctgagaggggtttttgcagccgcccaccgtctggtctacggtaccaaggatgactctggtcaggggtacctgacctggtctggacacagtgcccgcgtcggagccgcgcgggatatggcccgcgctggggtttcaatagcggagattatgcaggctggtggctggaccaccgtagagagcgttatgagctatctccgtaacctggatagtgagacaggtgcaatggtgcgcctgctggaggatggcgattag

SEQ ID NO: 32 - DNA, 1032 bp, synthetic construct
  misc_feature 1..1032: note = mutaed sequence
  source 1..1032: mol_type = other DNA
atgtccaatctacagaccctacaccagaatttgtccgcactactggccaatgcaacaagtgatgaggctcgcaagaacctgatggacgtcttcagggaccgccgggcgttttctgaagctacctggaaaacgcttctgtccgtttgccggacgtgggcggcatggtgcaagctgaataaccggaaatggtttcccgcagaacctgaagatgtacgcgattatcttctacatctccaggtacgcggtctggcggtgaacacaatccagcgacacttggccctgctaaacatgcttcaccgtcggtccgggctgccgcgaccaggtgacagcagcgctgtttcactggttatgcggcgtatccgaaaggaaaacgttgatgccggtgaacgcgtaaggcaggccctggcgttcgagcgcactgatttcgacaaggttcgttcactcatgggaaacagcgatcgctgccaggatatacgtaatctggcatttctgggtgttgcatataacaccctgctacgcatatccgaaattgccaggattcggataaaagatatctcacgtaccgacggtgggaggatgctaatccatatcggcagaacgcgcacgctggttagcaccgcaggcgtagagaaggcacttagccttggggtaaccagactggtcgggcggtggatttctgtctctggtgtagctggcgacccgaataactacctgttctgccgggtcagaaaaaatggtgttgccgcgccatctgccaccagccagctatcgaccgacgtcctgagaggggtttttgcagccgcccaccgtctggtctacggtaccaaggatgactctggtcaggggtacctgacctggtctggacacagtgcccgcgtcggagccgcgcgggatatggcccgcgctggggtttcaatagcggagattatgcaggctggtggctggaccaccgtagagagcgttatgagctatctccgtaacctggatagtgagacaggtgcaatggtgcgcctgctggaggatggcgattag

SEQ ID NO: 33 - DNA, 1032 bp, synthetic construct
  misc_feature 1..1032: note = mutated sequence
  source 1..1032: mol_type = other DNA
atgtccaatctacagaccctacaccagaatttgtccgcactactggccaatgcaacaagtgatgaggctcgcaagaacctgatggacgtcttcagggaccgccgggcgttttctgaagctacctggaaaacgcttctgtccgtttgccggacgtgggcggcatggtgcaagctgaataaccggaaatggtttcccgcagaacctgaagatgtacgcgattatcttctacatctccaggtacgcggtctggcggtgaacacaatccagcgacacttggccctgctaaacatgcttcaccgtcggtccgggctgccgcgaccaggtgacagcagcgctgtttcactggttatgcggcgtatccgaaaggaaaacgttgatgccggtgaacgcgtaaggcaggccctggcgttcgagcgcactgatttcgacaaggttcgttcactcatgggaaacagcgatcgctgccaggatatacgtaatctggcatttctgggtgttgcatataacaccctgctacgcatatccgaaattgccaggattcggataaaagatatctcacgtaccgacggtgggaggatgctaatccatatcggcagaacgaaaacgctggttagcaccgcaggcgtagagaaggcacttagccttggggtaaccagactggtcgggcggtggatttctgtctctggtgtagctggcgacccgaataactacctgttctgccgggtcagaaaaaatggtgttgccgcgccatctgccaccagccagctatcgaccgacgtcctgagaggggtttttgcagccgcccaccgtctggtctacggtaccaaggatgactctggtcaggaatacctgacctggtctggacacagtgcccgcgtcggagccgcgcgggatatggcccgcgctggggtttcaatagcggagattatgcaggctggtggctggaccaccgtagagagcgttatgagctatctccgtaacctggatagtgagacaggtgcaatggtgcgcctgctggaggatggcgattag

SEQ ID NO: 34 - DNA, 1032 bp, synthetic construct
  misc_feature 1..1032: note = recombinant sequence
  source 1..1032: mol_type = other DNA
atgtcaaacctgcaaactttacatcaaaacctgtcagccttgttagcaaacgcgacttctgacgaagcccgtcgtaatttgatgcgtgtgtttgagctgcgtcgtgcattctcagaggccacttggaagactctgcttagcgtgtgtcgcacctgggccgcgtggtgtaaattgaacaatcgcaagtggttcccggctgagccagaggacgtccgtgactacttgttgcacttacaagttcgtggcctcgcagttaataccattcaacgccatttagcattgctgaatatgctgcatcgccgcagcggtttgcctcgcccgggcgattcaagtgcggtgagtttggtgatgcgccgcattcgtaaactgaatgtggacgcaggggagcgtgtccgccaagcgctcgcttttgaacgtaccgactttgataaagtgcgcagtctgatgggcaattcggaccgttgtcaagacattcgcaacttagcgttcttgggcgtcgcgtacaatacactcctgcgtattagcgagatcgcacgcatccgtattaaggacattagccgcacagatggcggtcgtatgctgattcacattgggcgtaccaagaccttagtgtcgacggctggtgttgagaaagcgttaagtctgggtgtcacacgcttagtgggacgctggatctccgttagcggagttgcaggtgatcctaacaattatttattttgtcgtgttcgcaagaacggggtcgcagcaccgagcgcgacttcacaactcagcactgatgtacttcgcggtgtgttcgcggcggctcatcgcttagtttatggaacgaaagacgatagtggccaaggttatttaacttggagtggccatagcgcacgtgttggcgcggcccgcgacatggcgcgtgcaggtgtgagcattgctgaaatcatgcaagcgggcggatggactacagtggaatcggtgatgagttacctgcgcaatttagactcggaaactggggcgatggtagagcttttagaagacggtgactga

SEQ ID NO: 35 - DNA, 1032 bp, synthetic construct
  misc_feature 1..1032: note = rceombinant sequence
  source 1..1032: mol_type = other DNA
atgtccaatatacagaccccacaccaaagtttgtctgcattactgactgatgcaacgagtgatgtgactcgcaagaacctggcggacatgttcagggatagccaggcgttttcagagcatacctggaaaatgctcctgtccgtttgccggtcgtgggcggcatggtgcgagctgaataaccggaaatggcttcccgtagaacctgaggatgttcgcgattacctcctgcaccttcagacgcgtggtctggcggtaaaaactatccagcatcatctgggctcgctaaacatgcttcaccgtcgggctgggctgccacgaccgggtgacagcaatgctgtttcactggttatgcggcgtatccgaagagaaaacgttgatgccggtgaacgcgcacaacaggccctggcgttcgaacgcaccgatttcgaccaggttcggtcactcgtggaaaacagcgaccgctgccaggatatacgtaatctggcatttctgggggtcgcttataacaccctgttgcgtatatccgaaattgccaggattcgggttaaggatatctcacgtactgacggtgggaggatgttaatccatattggcagaacgaaaacgctggttagtaccgcaggtgtagagaaggcactcagcctgggggtgaccaaactggtcgagcgatggatttccgtctccggtgtagccgatgacccaaataactacctgttttgccgggtcagaagatatggcgttgccaagccgtctgccaccagccagctgtcaacttacgccctgcaagggattttcggagccgcccaccgcctggtctacggtgccaagggtgactctggccagaagtacctggcctggtcgggacacagtgcccgtgtcggagccgcacgagacatggcccgcgctggagttccaataccggagattatgcaggctggtggctggaccaccgtgaatagtgtcatgaactatatccgtaacctggatagcgagacaggagcaatggtgcgcctgctggaagacagcgattag

SEQ ID NO: 36 - DNA, 1032 bp, synthetic construct
  misc_feature 1..1032: note = mutated sequence
  source 1..1032: mol_type = other DNA
atgtccaatatacagaccccacaccaaagtttgtctgcattactgactgatgcaacgagtgatgtgactcgcaagaacctggcggacatgttcagggatagccaggcgttttcagagcatacctggaaaatgctcctgtccgtttgccggtcgtgggcggcatggtgcgagctgaataaccggaaatggcttcccgtagaacctgaggatgttcgcgattacctcctgcaccttcagacgcgtggtctggcggtaaaaactatccagcatcatctgggctcgctaaacatgcttcaccgtcgggctgggctgccacgaccgggtgacagcaatgctgtttcactggttatgcggcgtatccgaagagaaaacgttgatgccggtgaacgcgcacaacaggccctggcgttcgaacgcaccgatttcgaccaggttcggtcactcgtggaaaacagcgaccgctgccaggatatacgtaatctggcatttctgggggtcgcttataacaccctgttgcgtatatccgaaattgccaggattcgggttaaggatatctcacgtactgacggtgggaggatgttaatccatattggcagaacgaaaacgctggttagtaccgcaggtgtagagaaggcactcagcctgggggtgaccaaactggtcgagcgatggatttccgtctccggtgtagccgatgacccaaataactacctgttttgccgggtcagaagatatggcgttgccaagccgtctgccaccagccagctgtcaacttacgccctgcaagggattttcggagccgcccaccgcctggtctacggtgccaagggtgactctggccagaagtacctggcctggtcgggacacagtgcccgtgtcggagccgcacgagacatggcccgcgctggagttccaataccggagattatgcgcgctggtggctggaccaccgtgaatagtgtcatgaactatatccgtaacctggatagcgagacaggagcaatggtgcgcctgctggaagacagcgattag

SEQ ID NO: 37 - DNA, 1032 bp, synthetic construct
  misc_feature 1..1032: note = mutated sequence
  source 1..1032: mol_type = other DNA
atgtccaatatacagaccccacaccaaagtttgtctgcattactgactgatgcaacgagtgatgtgactcgcaagaacctggcggacatgttcagggatagccaggcgttttcagagcatacctggaaaatgctcctgtccgtttgccggtcgtgggcggcatggtgcgagctgaataaccggaaatggcttcccgtagaacctgaggatgttcgcgattacctcctgcaccttcagacgcgtggtctggcggtaaaaactatccagcatcatctgggctcgctaaacatgcttcaccgtcgggctgggctgccacgaccgggtgacagcaatgctgtttcactggttatgcggcgtatccgaagagaaaacgttgatgccggtgaacgcgcacaacaggccctggcgttcgaacgcaccgatttcgaccaggttcggtcactcgtggaaaacagcgaccgctgccaggatatacgtaatctggcatttctgggggtcgcttataacaccctgttgcgtatatccgaaattgccaggattcgggttaaggatatctcacgtactgacggtgggaggatgttaatccatattggcagaacgaaaacgctggttagtaccgcaggtgtagagaaggcactcagcctgggggtgaccaaactggtcgagcgatggatttccgtctccggtgtagccgatgacccaaataactacctgttttgccgggtcagaagatatggcgttgccaagccgtctgccaccagccagctgtcaacttacgccctgcaagggattttcggagccgcccaccgcctggtctacggtgccaagggtgactctggccagaagtacctggcctggtcgggacacagtgcccgtgtcggagccgcacgagacatggcccgcgctggagttccaataccggagattatgaaagctggtggctggaccaccgtgaatagtgtcatgaactatatccgtaacctggatagcgagacaggagcaatggtgcgcctgctggaagacagcgattag

SEQ ID NO: 38 - DNA, 1032 bp, synthetic construct
  misc_feature 1..1032: note = recombinant sequence
  source 1..1032: mol_type = other DNA
atgtcaaacatccaaacaccgcatcagtcgcttagcgcgctgttgacggacgccacttcagacgtcacgcgtaagaatttagctgatatgtttcgcgactctcaagcattcagcgaacacacttggaagatgctgctctcggtgtgtcgctcctgggccgcgtggtgtgaacttaacaatcgcaagtggttgccggttgagccggacgacgtgaaggactatttagagcatctgcaaacacgcggcttggcagtgaagacgattcaacaccacttaggatcactgaatatgttacatcgccgtgcaggtcttccgcgccccggagattcgaacgccgtgtccctcgttatgcgtcgcattcgtcgtgagaatgtagacgcgggtgagcgtgcccagcaagcgcttgcctttgagcgtactgactttgatcaagtgcgttccctggttgagaatagtgatcgttgtcaagacattcgcaacttggcgttcttgggcgttgcctacaatacgctcctgcgcattagcgagatcgcgcgcatccgtgtaaaagacattagtcgcaccgatggcggtcgcatgttgattcacatcgggcgcaccaagacccttgtctcgactgcgggcgtggagaaagcgttatctttaggcgtcacgaagttagttgaacgttggatctcggttagcggagtcgcagacgatccgaacaattatttattctgtcgcgtgcgccgctacggggtcgctaaaccttcggcgacgagtcaactttctacctatgcgttacagggaatctttggcgctgctcatcgtttagtgtatggcgcgaaaggggattccgggcaaaagtatcttgcatggagtgggcattcagctcgcgtgggtgcagcccgtgatatggctcgtgcgggtgtgcctatccctcgcatcatgcaagcgggcggttggacgacggtcaacagcgtgatgaattacattcgcaatctcgactcggaaaccggcgccatggtccgtctcttagaggattctgactaa

SEQ ID NO: 39 - DNA, 1086 bp, Vibrio coralliilyticus
  source 1..1086: mol_type = other DNA
atgaccgatctgaccccgtttccgcctctggaacatctggaaccggatgaatttgccgatctggttcgtaaagcaattaaacgtgatccgcaggcaggcgcacatccggcaattcagagcgcaattagccattttcaggatgaatttgttcgtcgtcagggtgaatggcagccagcaaccctgcagcgtctgcgtaatgcatggaatgtttttgttcgttggtgtacccatcagggtattccggcactgcctgcacgtcatcaggatgttgaacgttatctgattgaacgtcgcaatgaactgcatcgtaataccctgaaagttcatctgtgggcaattggtaaaacccatgttattagcggtctgccgaatccgtgtgcacatcgttatgttaaagcacagatggcacagattacccatcagaaagttcgtgaacgtgaacgtattgaacaggcaccggcatttcgtgaaagcgatctggatcgtctgaccgaactgtggtcagcaacccgtagcgttacccagcagcgtgatctgatgattgttagcctggcttacgaaaccctgctgcgtaaaaacaacctggaacaaatgaaagtgggcgatattgaattttgtcaggatggtagcgcactgattaccattccgtttagcaaaaccaatcatagcggtcgtgatgatgttcgttggattagtccgcaggttgcaaatcaggttcatgcatatctgcagctgccgaatattgatgcagatccgcagtgttttctgctgcagcgtgttaaacgtagcggtaaagcactgaatccggaaagccataacaccctgaatggtcatcatccggttagcgaaaaactgattagccgtgtttttgaacgtgcatggcgtgcactgaatcatgaaaccggtccgcgttataccggtcatagcgcacgtgttggtgcagcacaggatctgctgcaagaaggttatagcaccctgcaggttatgcaggcaggcggttggagcagcgaaaaaatggttctgcgttatggtcgtcatctgcatgcacataccagcgcaatggcacagaaacgtcgtcagcgttaa

SEQ ID NO: 40 - DNA, 1086 bp, synthetic construct
  misc_feature 1..1086: note = mutated sequence
  source 1..1086: mol_type = other DNA
atgaccgatctgaccccgtttccgcctctggaacatctggaaccggatgaatttgccgatctggttcgtaaagcaattaaacgtgatccgcaggcaggcgcacatccggcaattcagagcgcaattagccattttcaggatgaatttgttcgtcgtcagggtgaatggcagccagcaaccctgcagcgtctgcgtaatgcatggaatgtttttgttcgttggtgtacccatcagggtattccggcactgcctgcacgtcatcaggatgttgaacgttatctgattgaacgtcgcaatgaactgcatcgtaataccctgaaagttcatctgtgggcaattggtaaaacccatgttattagcggtctgccgaatccgtgtgcacatcgttatgttaaagcacagatggcacagattacccatcagaaagttcgtgaacgtgaacgtattgaacaggcaccggcatttcgtgaaagcgatctggatcgtctgaccgaactgtggtcagcaacccgtagcgttacccagcagcgtgatctgatgattgttagcctggcttacgaaaccctgctgcgtaaaaacaacctggaacaaatgaaagtgggcgatattgaattttgtcaggatggtagcgcactgattaccattccgtttagccgcaccaatcatagcggtcgtgatgatgttcgttggattagtccgcaggttgcaaatcaggttcatgcatatctgcagctgccgaatattgatgcagatccgcagtgttttctgctgcagcgtgttaaacgtagcggtaaagcactgaatccggaaagccataacaccctgaatggtcatcatccggttagcgaaaaactgattagccgtgtttttgaacgtgcatggcgtgcactgaatcatgaaaccggtccgcgttataccggtcatagcgcacgtgttggtgcagcacaggatctgctgcaagaaggttatagcaccctgcaggttatgcaggcaggcggttggagcagcgaaaaaatggttctgcgttatggtcgtcatctgcatgcacataccagcgcaatggcacagaaacgtcgtcagcgttaa

SEQ ID NO: 41 - DNA, 1086 bp, synthetic construct
  misc_feature 1..1086: note = mutaed sequence
  source 1..1086: mol_type = other DNA
atgaccgatctgaccccgtttccgcctctggaacatctggaaccggatgaatttgccgatctggttcgtaaagcaattaaacgtgatccgcaggcaggcgcacatccggcaattcagagcgcaattagccattttcaggatgaatttgttcgtcgtcagggtgaatggcagccagcaaccctgcagcgtctgcgtaatgcatggaatgtttttgttcgttggtgtacccatcagggtattccggcactgcctgcacgtcatcaggatgttgaacgttatctgattgaacgtcgcaatgaactgcatcgtaataccctgaaagttcatctgtgggcaattggtaaaacccatgttattagcggtctgccgaatccgtgtgcacatcgttatgttaaagcacagatggcacagattacccatcagaaagttcgtgaacgtgaacgtattgaacaggcaccggcatttcgtgaaagcgatctggatcgtctgaccgaactgtggtcagcaacccgtagcgttacccagcagcgtgatctgatgattgttagcctggcttacgaaaccctgctgcgtaaaaacaacctggaacaaatgaaagtgggcgatattgaattttgtcaggatggtagcgcactgattaccattccgtttagcaaaaccaatcatagcggtcgtgatgatgttcgttggattagtccgcaggttgcaaatcaggttcatgcatatctgcagctgccgaatattgatgcagatccgcagtgttttctgctgcagcgtgttaaacgtagcggtaaagcactgaatccggaaagccataacaccctgaatggtcatcatccggttagcgaaaaactgattagccgtgtttttgaacgtgcatggcgtgcactgaatcatgaaaccggtccgcgttataccggtcatagcgcacgtgttggtgcagcacaggatctgctgcaagaaggttatagcaccctgcaggttatgcgcgcaggcggttggagcagcgaaaaaatggttctgcgttatggtcgtcatctgcatgcacataccagcgcaatggcacagaaacgtcgtcagcgttaa

SEQ ID NO: 42 - DNA, 1032 bp, unidentified
  source 1..1032: mol_type = other DNA
atgaaacatctggcgcagattaaaaccggcgcgctgccggcggaactgacccagctgaccccggaagatattgcgaacaacctgcgccgctttattgcggataaagcggcgtatagcgaaaacacctttcgcgatctgctgagcgtgattcgccgctgggcgttttggtgcaacgaacgcgatgtgggctatctgccgattgatccggaactggcgcgcgaatattttctgcagatggcggaaagcggcctggcgagcagcaccattgataaacattatgcgatgatgaacatgctgtgccgcgaaagcggcctgccggatctgcgcggcagcgtggatctgaaacgcagcatgaaacgcattcgccgcgaagcggtgctgcagggcgaacgcaccggccaggcggtgccgtttcgcctgccggatctgcagctgctgagccatctgatgggccgcagcgatcgcctgaccgatcagcgcaacctggcgtttctgtttgtggcgtataacaccctgtgccgcatgagcgaactgagccgcattcgcgtgcgcgatctggatattagcgatagcggccatgtggtgattaacctgagccataccaaaaccatggtgaccgcggcgggcgtgattaaacatctgagccgcgcggcggcgggccatctgatgcattggctggaactgagcggcctgattcatcatccggatgcgatggtgtttggcccggtgcgccataacaacaccgcgggcgtgagcgaaaaaccgatgagcgcgccggcgaccgaaaaaatttttaaagatgcgtgggatctgctgggcaaagaaccggtgcaggataacaaaggccgctatgcgaaatggagcggccatagcgcgcgcgtgggcgcggcgatggatatggcggaacgcgatgcgaccattacccagattatgcaggaaggcacctggcaggatccgaaaaccgtgatgcgctatctgcgccgcagcgaaagccagaaaggcaaaatgagcggcattctggatggcgaatag

SEQ ID NO: 43 - DNA, 1032 bp, synthetic construct
  misc_feature 1..1032: note = mutated sequence
  source 1..1032: mol_type = other DNA
atgaaacatctggcgcagattaaaaccggcgcgctgccggcggaactgacccagctgaccccggaagatattgcgaacaacctgcgccgctttattgcggataaagcggcgtatagcgaaaacacctttcgcgatctgctgagcgtgattcgccgctgggcgttttggtgcaacgaacgcgatgtgggctatctgccgattgatccggaactggcgcgcgaatattttctgcagatggcggaaagcggcctggcgagcagcaccattgataaacattatgcgatgatgaacatgctgtgccgcgaaagcggcctgccggatctgcgcggcagcgtggatctgaaacgcagcatgaaacgcattcgccgcgaagcggtgctgcagggcgaacgcaccggccaggcggtgccgtttcgcctgccggatctgcagctgctgagccatctgatgggccgcagcgatcgcctgaccgatcagcgcaacctggcgtttctgtttgtggcgtataacaccctgtgccgcatgagcgaactgagccgcattcgcgtgcgcgatctggatattagcgatagcggccatgtggtgattaacctgagccatacccgcaccatggtgaccgcggcgggcgtgattaaacatctgagccgcgcggcggcgggccatctgatgcattggctggaactgagcggcctgattcatcatccggatgcgatggtgtttggcccggtgcgccataacaacaccgcgggcgtgagcgaaaaaccgatgagcgcgccggcgaccgaaaaaatttttaaagatgcgtgggatctgctgggcaaagaaccggtgcaggataacaaaggccgctatgcgaaatggagcggccatagcgcgcgcgtgggcgcggcgatggatatggcggaacgcgatgcgaccattacccagattatgcaggaaggcacctggcaggatccgaaaaccgtgatgcgctatctgcgccgcagcgaaagccagaaaggcaaaatgagcggcattctggatggcgaatag

SEQ ID NO: 44 - DNA, 1032 bp, synthetic construct
  misc_feature 1..1032: note = mutated sequence
  source 1..1032: mol_type = other DNA
atgaaacatctggcgcagattaaaaccggcgcgctgccggcggaactgacccagctgaccccggaagatattgcgaacaacctgcgccgctttattgcggataaagcggcgtatagcgaaaacacctttcgcgatctgctgagcgtgattcgccgctgggcgttttggtgcaacgaacgcgatgtgggctatctgccgattgatccggaactggcgcgcgaatattttctgcagatggcggaaagcggcctggcgagcagcaccattgataaacattatgcgatgatgaacatgctgtgccgcgaaagcggcctgccggatctgcgcggcagcgtggatctgaaacgcagcatgaaacgcattcgccgcgaagcggtgctgcagggcgaacgcaccggccaggcggtgccgtttcgcctgccggatctgcagctgctgagccatctgatgggccgcagcgatcgcctgaccgatcagcgcaacctggcgtttctgtttgtggcgtataacaccctgtgccgcatgagcgaactgagccgcattcgcgtgcgcgatctggatattagcgatagcggccatgtggtgattaacctgagccataccaaaaccatggtgaccgcggcgggcgtgattaaacatctgagccgcgcggcggcgggccatctgatgcattggctggaactgagcggcctgattcatcatccggatgcgatggtgtttggcccggtgcgccataacaacaccgcgggcgtgagcgaaaaaccgatgagcgcgccggcgaccgaaaaaatttttaaagatgcgtgggatctgctgggcaaagaaccggtgcaggataacaaaggccgctatgcgaaatggagcggccatagcgcgcgcgtgggcgcggcgatggatatggcggaacgcgatgcgaccattacccagattatgcgcgaaggcacctggcaggatccgaaaaccgtgatgcgctatctgcgccgcagcgaaagccagaaaggcaaaatgagcggcattctggatggcgaatag

SEQ ID NO: 45 - DNA, 1029 bp, unidentified
  source 1..1029: mol_type = other DNA
atgtctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtgtctatggtcgagattatgcaggaagggacatggaagaagcctgaaacactgatgcggtatctcagaaggggcggagtgtccgtgggagccaattctcgactgatggatagctag

SEQ ID NO: 46 - DNA, 1029 bp, synthetic construct
  misc_feature 1..1029: note = mutated sequence
  source 1..1029: mol_type = other DNA
atgtctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactcgcacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtgtctatggtcgagattatgcaggaagggacatggaagaagcctgaaacactgatgcggtatctcagaaggggcggagtgtccgtgggagccaattctcgactgatggatagctag

SEQ ID NO: 47 - DNA, 1029 bp, synthetic construct
  misc_feature 1..1029: note = mutated sequence
  source 1..1029: mol_type = other DNA
atgtctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtgtctatggtcgagattatgcgcgaagggacatggaagaagcctgaaacactgatgcggtatctcagaaggggcggagtgtccgtgggagccaattctcgactgatggatagctag

SEQ ID NO: 48 - DNA, 1143 bp, unidentified
  source 1..1143: mol_type = other DNA
atgatagagaatcagctaagcttactgggcgatttttctggagtacgccctgacgatgtaaaaacggctatccaggctgcgcagaaaaaaggaattaatgtcgccgaaaatgaacagtttaaagccgcgtttgagcacctgctgaatgagttcaagaaacgcgaggaacgttatagtccaaatactctcaggcggttagagtcggcgtggacttgctttgtggattggtgtcttgccaaccatcgacattctctgccggcaactccagatacagtagaagcgttcttcattgagagagcagaggagcttcatcgtaacaccttatcagtttatcgttgggcaatttcgcgggtgcaccgcgtagccggttgccctgatccgtgtctcgatatatatgtagaagatcgtctaaaggctatagctcggaagaaagtgcgcgaaggtgaagctgtcaaacaggctagtccctttaacgagcagcaccttctgaagctgactagtctgtggtatcgaagtgataaactcctgcttaggcgcaatttagcattgctggccgtggcttacgagtctatgttacgggcatcggaactggcgaacatccgagtgtccgatatggaacttgctggagatggtacagcgatccttaccattcctattacgaaaacaaatcactccggtgaacccgacacatgtattctctcacaggacgttgtctctctgctgatggactatacggaggcaggcaaactcgatatgtctagcgacggttttctatttgttggggtcagtaaacataatacgtgtatcaaaccgaagaaggataaacaaacaggcgaagtgcttcacaaacctattacgacaaagacggtagaaggagtattctattcggcatgggaaaccttagacttgggaaggcaaggggtgaaaccttttactgcacactctgcccgtgtgggagccgcacaggatctgctgaagaagggctataacaccttacagatccagcagagcgggagatggagttctggcgcaatggtggcccgttacggtcgtgctattctggcccgagatggcgccatggcacattctcgggtaaaaacacgttccgcgccaatgcaatggggcaaagatgaaaaggactag

SEQ ID NO: 49 - DNA, 1143 bp, synthetic construct
  misc_feature 1..1143: note = mutated sequence
  source 1..1143: mol_type = other DNA
atgatagagaatcagctaagcttactgggcgatttttctggagtacgccctgacgatgtaaaaacggctatccaggctgcgcagaaaaaaggaattaatgtcgccgaaaatgaacagtttaaagccgcgtttgagcacctgctgaatgagttcaagaaacgcgaggaacgttatagtccaaatactctcaggcggttagagtcggcgtggacttgctttgtggattggtgtcttgccaaccatcgacattctctgccggcaactccagatacagtagaagcgttcttcattgagagagcagaggagcttcatcgtaacaccttatcagtttatcgttgggcaatttcgcgggtgcaccgcgtagccggttgccctgatccgtgtctcgatatatatgtagaagatcgtctaaaggctatagctcggaagaaagtgcgcgaaggtgaagctgtcaaacaggctagtccctttaacgagcagcaccttctgaagctgactagtctgtggtatcgaagtgataaactcctgcttaggcgcaatttagcattgctggccgtggcttacgagtctatgttacgggcatcggaactggcgaacatccgagtgtccgatatggaacttgctggagatggtacagcgatccttaccattcctattacgcgcacaaatcactccggtgaacccgacacatgtattctctcacaggacgttgtctctctgctgatggactatacggaggcaggcaaactcgatatgtctagcgacggttttctatttgttggggtcagtaaacataatacgtgtatcaaaccgaagaaggataaacaaacaggcgaagtgcttcacaaacctattacgacaaagacggtagaaggagtattctattcggcatgggaaaccttagacttgggaaggcaaggggtgaaaccttttactgcacactctgcccgtgtgggagccgcacaggatctgctgaagaagggctataacaccttacagatccagcagagcgggagatggagttctggcgcaatggtggcccgttacggtcgtgctattctggcccgagatggcgccatggcacattctcgggtaaaaacacgttccgcgccaatgcaatggggcaaagatgaaaaggactag

SEQ ID NO: 50 - DNA, 1143 bp, synthetic construct
  misc_feature 1..1143: note = mutated sequence
  source 1..1143: mol_type = other DNA
atgatagagaatcagctaagcttactgggcgatttttctggagtacgccctgacgatgtaaaaacggctatccaggctgcgcagaaaaaaggaattaatgtcgccgaaaatgaacagtttaaagccgcgtttgagcacctgctgaatgagttcaagaaacgcgaggaacgttatagtccaaatactctcaggcggttagagtcggcgtggacttgctttgtggattggtgtcttgccaaccatcgacattctctgccggcaactccagatacagtagaagcgttcttcattgagagagcagaggagcttcatcgtaacaccttatcagtttatcgttgggcaatttcgcgggtgcaccgcgtagccggttgccctgatccgtgtctcgatatatatgtagaagatcgtctaaaggctatagctcggaagaaagtgcgcgaaggtgaagctgtcaaacaggctagtccctttaacgagcagcaccttctgaagctgactagtctgtggtatcgaagtgataaactcctgcttaggcgcaatttagcattgctggccgtggcttacgagtctatgttacgggcatcggaactggcgaacatccgagtgtccgatatggaacttgctggagatggtacagcgatccttaccattcctattacgaaaacaaatcactccggtgaacccgacacatgtattctctcacaggacgttgtctctctgctgatggactatacggaggcaggcaaactcgatatgtctagcgacggttttctatttgttggggtcagtaaacataatacgtgtatcaaaccgaagaaggataaacaaacaggcgaagtgcttcacaaacctattacgacaaagacggtagaaggagtattctattcggcatgggaaaccttagacttgggaaggcaaggggtgaaaccttttactgcacactctgcccgtgtgggagccgcacaggatctgctgaagaagggctataacaccttacagatccagcgcagcgggagatggagttctggcgcaatggtggcccgttacggtcgtgctattctggcccgagatggcgccatggcacattctcgggtaaaaacacgttccgcgccaatgcaatggggcaaagatgaaaaggactag

SEQ ID NO: 51 - DNA, 34 bp, unidentified
  source 1..34: mol_type = other DNA
ataacttcgtataatgtatgctatacgaagttat

SEQ ID NO: 52 - DNA, 32 bp, unidentified
  source 1..32: mol_type = other DNA
taactttaaataatgccaattatttaaagtta

SEQ ID NO: 53 - DNA, 34 bp, unidentified
  source 1..34: mol_type = other DNA
tcaatttctgagaatgacagttctcggaaattga

SEQ ID NO: 54 - DNA, 34 bp, unidentified
  source 1..34: mol_type = other DNA
ctcgtgtccgataactgtaattatcggacatgat

SEQ ID NO: 55 - DNA, 34 bp, unidentified
  source 1..34: mol_type = other DNA
aataggtctgagaatgggcgttctcagacgtatt

SEQ ID NO: 56 - DNA, 21 bp, unidentified
  source 1..21: mol_type = other DNA
cagcttttttatactaagttg

SEQ ID NO: 57 - DNA, 34 bp, unidentified
  source 1..34: mol_type = other DNA
gaagttcctatactttctagagaataggaacttc

SEQ ID NO: 58 - DNA, 31 bp, unidentified
  source 1..31: mol_type = other DNA
ttgatgaaagaataacgtattctttcatcaa

SEQ ID NO: 59 - DNA, 43 bp, unidentified
  source 1..43: mol_type = other DNA
acgaaaaatggtaaggaatagaccattccttaccatttttcgt

SEQ ID NO: 60 - DNA, 61 bp, unidentified
  source 1..61: mol_type = other DNA
aaacgatatcagacatttgtctgataatgcttcattatcagacaaatgtctgatatcgttt

SEQ ID NO: 61 - DNA, 39 bp, unidentified
  source 1..39: mol_type = other DNA
gagtttcattaaggaataactaattccctaatgaaactc

SEQ ID NO: 62 - DNA, 33 bp, unidentified
  source 1..33: mol_type = other DNA
ggttgcttaagaataagtaattcttaagcaacc

SEQ ID NO: 63 - DNA, 34 bp, unidentified
  source 1..34: mol_type = other DNA
tgaatgtcctataattacacttataggacattca

SEQ ID NO: 64 - DNA, 34 bp, unidentified
  source 1..34: mol_type = other DNA
gaaactttaaataataagtcttatttaaagtttc

SEQ ID NO: 65 - DNA, 34 bp, unidentified
  source 1..34: mol_type = other DNA
ataaatctgtggaaacgctgccacacaatcttag

SEQ ID NO: 66 - DNA, 34 bp, unidentified
  source 1..34: mol_type = other DNA
ctaagattgtgtggcagcgtttccacagatttat

SEQ ID NO: 67 - DNA, 34 bp, unidentified
  source 1..34: mol_type = other DNA
ataaatctgtggaaacgctgctccacagatttat

SEQ ID NO: 68 - DNA, 34 bp, unidentified
  source 1..34: mol_type = other DNA
ctaagattgtgtgaacgctgccacacaatcttag

SEQ ID NO: 69 - DNA, 34 bp, unidentified
  source 1..34: mol_type = other DNA
ataaatttgtggaaattaaacaacacactcttaa

SEQ ID NO: 70 - DNA, 34 bp, unidentified
  source 1..34: mol_type = other DNA
acaaaattgtggaaattaaacaacacaatcttaa

SEQ ID NO: 71 - DNA, 34 bp, unidentified
  source 1..34: mol_type = other DNA
acaaatatgtggaaattaaacaacacactcttga

SEQ ID NO: 72 - DNA, 34 bp, unidentified; identical to SEQ ID NO: 69
  source 1..34: mol_type = other DNA

SEQ ID NO: 73 - DNA, 34 bp, unidentified
  source 1..34: mol_type = other DNA
ttaagagtgtgttttttaatttccacatatttgt

SEQ ID NO: 74 - DNA, 34 bp, unidentified
  source 1..34: mol_type = other DNA
ataaatatgtgaatatacatattcacatatttat

SEQ ID NO: 75 - DNA, 34 bp, unidentified
  source 1..34: mol_type = other DNA
atatatctatagatatagatatccacagatatat

SEQ ID NO: 76 - DNA, 34 bp, unidentified
  source 1..34: mol_type = other DNA
ctatgtttttgaggtcttattcacaaaatctttt

SEQ ID NO: 77 - DNA, 34 bp, unidentified
  source 1..34: mol_type = other DNA
ctattattgtgtaacaaattacccccaaacttag

SEQ ID NO: 78 - DNA, 34 bp, unidentified
  source 1..34: mol_type = other DNA
ataaatatgtgtgtatatatacacacaaacatat

SEQ ID NO: 79 - DNA, 34 bp, unidentified
  source 1..34: mol_type = other DNA
atatatctgtgtatatatatacacacacacatat

SEQ ID NO: 80 - DNA, 34 bp, unidentified
  source 1..34: mol_type = other DNA
atatatatgtgtatatatatacacacatacatat

SEQ ID NO: 81 - DNA, 34 bp, unidentified
  source 1..34: mol_type = other DNA
atatatgtgtgtatatatatacacacacacatac

SEQ ID NO: 82 - DNA, 34 bp, unidentified
  source 1..34: mol_type = other DNA
ataaatatgtgtaaactaaacaacacactcttaa

SEQ ID NO: 83 - DNA, 34 bp, unidentified
  source 1..34: mol_type = other DNA
acaaatatgtggaaactaaacaacacattcttga

SEQ ID NO: 84 - DNA, 34 bp, unidentified; identical to SEQ ID NO: 51
  source 1..34: mol_type = other DNA

SEQ ID NO: 85 - DNA, 20 bp, synthetic construct
  misc_feature 1..20: note = Primer
  source 1..20: mol_type = other DNA
tgcatcagacattgccgtca

SEQ ID NO: 86 - DNA, 20 bp, synthetic construct
  misc_feature 1..20: note = Primer
  source 1..20: mol_type = other DNA
agaccgcttctgcgttctga

SEQ ID NO: 87 - DNA, 20 bp, synthetic construct
  misc_feature 1..20: note = Primer
  source 1..20: mol_type = other DNA
aagggaataagggcgacacg

SEQ ID NO: 88 - DNA, 21 bp, synthetic construct
  misc_feature 1..21: note = Primer
  source 1..21: mol_type = other DNA
ctaactgacacacattccaca

SEQ ID NO: 89 - DNA, 27 bp, synthetic construct
  misc_feature 1..27: note = Primer
  misc_feature 17: note = n is a, c, g, or t
  source 1..27: mol_type = other DNA
acgcgtctgaaggtgsnbgaggtaatc

SEQ ID NO: 90 - DNA, 27 bp, synthetic construct
  misc_feature 1..27: note = Primer
  misc_feature 17: note = n is a, c, g, or t
  misc_feature 20: note = n is a, c, g, or t
  source 1..27: mol_type = other DNA
agaaaacgcccggcgsnbsnbgaagac

SEQ ID NO: 91 - DNA, 36 bp, synthetic construct
  misc_feature 1..36: note = Primer
  misc_feature 17: note = n is a, c, g, or t
  misc_feature 29: note = n is a, c, g, or t
  source 1..36: mol_type = other DNA
agtgatgaggctcgcvnsaacctgatgvnsgtcttc

SEQ ID NO: 92 - DNA, 30 bp, synthetic construct
  misc_feature 1..30: note = Primer
  misc_feature 11: note = n is a, c, g, or t
  misc_feature 20: note = n is a, c, g, or t
  source 1..30: mol_type = other DNA
gtagaacctvnsgatgttvnsgattacctc

SEQ ID NO: 93 - DNA, 33 bp, synthetic construct
  misc_feature 1..33: note = Primer
  source 1..33: mol_type = other DNA
atgtccaatctacagaccctacaccagaatttg

SEQ ID NO: 94 - DNA, 36 bp, synthetic construct
  misc_feature 1..36: note = Primer
  source 1..36: mol_type = other DNA
gaagacwdccatcaggttwdcgcgagcctcatcact

SEQ ID NO: 95 - DNA, 36 bp, synthetic construct
  misc_feature 1..36: note = Primer
  source 1..36: mol_type = other DNA
gaagacchkcatcaggttchkgcgagcctcatcact

SEQ ID NO: 96 - DNA, 33 bp, synthetic construct
  misc_feature 1..33: note = Primer
  source 1..33: mol_type = other DNA
gtcttcghwghwcgcghwgcgttttctgaagct

SEQ ID NO: 97 - DNA, 33 bp, synthetic construct
  misc_feature 1..33: note = Primer
  source 1..33: mol_type = other DNA
gtcttcmdgmdgcgcmdggcgttttctgaagct

SEQ ID NO: 98 - DNA, 33 bp, synthetic construct
  misc_feature 1..33: note = Primer
  source 1..33: mol_type = other DNA
attcagcttgcaccatgccgcccacgtccggca

SEQ ID NO: 99 - DNA, 33 bp, synthetic construct
  misc_feature 1..33: note = Primer
  source 1..33: mol_type = other DNA
ctgtccgtttgccggtcgtgggcggcatggtgc

SEQ ID NO: 100 - DNA, 33 bp, synthetic construct
  misc_feature 1..33: note = Primer
  source 1..33: mol_type = other DNA
gaggtaatcwdcaacatcwdcaggttctacggg

SEQ ID NO: 101 - DNA, 33 bp, synthetic construct
  misc_feature 1..33: note = Primer
  source 1..33: mol_type = other DNA
gaggtaatcchkaacatcchkaggttctacggg

SEQ ID NO: 102 - DNA, 33 bp, synthetic construct
  misc_feature 1..33: note = Primer
  source 1..33: mol_type = other DNA
gattacctcghwcaccttcagacgcgtggtctg

SEQ ID NO: 103 - DNA, 33 bp, synthetic construct
  misc_feature 1..33: note = Primer
  source 1..33: mol_type = other DNA
gattacctcmdgcaccttcagacgcgtggtctg

SEQ ID NO: 104 - DNA, 33 bp, synthetic construct
  misc_feature 1..33: note = Primer
  source 1..33: mol_type = other DNA
agcccgacggtgaagcatgtttagcgagcccag

SEQ ID NO: 105 - DNA, 84 bp, synthetic construct
  misc_feature 1..84: note = Primer
  source 1..84: mol_type = other DNA
gctaatacgactcactatagggagagccgccaccatgccaaaaaagaagagaaaggtaatgtccaatttactgaccgtacacca

SEQ ID NO: 106 - DNA, 46 bp, synthetic construct
  misc_feature 1..46: note = Primer
  source 1..46: mol_type = other DNA
ttttttttttttttttggtttattcctaatcgccatcttccagcag

SEQ ID NO: 107 - DNA, 20 bp, synthetic construct
  source 1..20: mol_type = other DNA
tgtatccgctcatgagacaa

SEQ ID NO: 108 - DNA, 19 bp, synthetic construct
  source 1..19: mol_type = other DNA
ttaaacgcctggttgctac

SEQ ID NO: 109 - protein, 8 aa, synthetic construct
ERAKQALA

SEQ ID NO: 110 - protein, 19 aa, synthetic construct
FLGIAYNTLLRIAEIARIR

SEQ ID NO: 111 - protein, 13 aa, synthetic construct
RTKTLVSTAGVEK

SEQ ID NO: 112 - protein, 13 aa, synthetic construct
HSARVGAARDMAR

SEQ ID NO: 113 - protein, 7 aa, synthetic construct
MQAGGWT

SEQ ID NO: 114 - protein, 4 aa, synthetic construct
VMNY

SEQ ID NO: 115 - protein, 8 aa, synthetic construct
ERIEQAPA

SEQ ID NO: 116 - protein, 19 aa, synthetic construct
IVSLAYETLLRKNNLEQMK

SEQ ID NO: 117 - protein, 13 aa, synthetic construct
FSKTNHSGRDDVR

SEQ ID NO: 118 - protein, 13 aa, synthetic construct
HSARVGAAQDLLQ

SEQ ID NO: 119 - protein, 7 aa, synthetic construct
MQAGGWS

SEQ ID NO: 120 - protein, 4 aa, synthetic construct
VLRY

SEQ ID NO: 121 - protein, 8 aa, synthetic construct
ERTGQAIP

SEQ ID NO: 122 - protein, 19 aa, synthetic construct
FLFVAYNTLMRMSEISRIR

SEQ ID NO: 123 - protein, 13 aa, synthetic construct
HTKTITTAAGLDK

SEQ ID NO: 124 - protein, 13 aa, synthetic construct
HSARVGAAIDMAE

SEQ ID NO: 125 - protein, 7 aa, synthetic construct
MQEGTWK

SEQ ID NO: 126 - protein, 4 aa, synthetic construct
LMRY

SEQ ID NO: 127 - protein, 8 aa, synthetic construct
ERTGQAVP

SEQ ID NO: 128 - protein, 19 aa, synthetic construct
FLFVAYNTLCRMSELSRIR

SEQ ID NO: 129 - protein, 13 aa, synthetic construct
HTKTMVTAAGVIK

SEQ ID NO: 130 - protein, 13 aa, synthetic construct
HSARVGAAMDMAE

SEQ ID NO: 131 - protein, 7 aa, synthetic construct
MQEGTWQ

SEQ ID NO: 132 - protein, 4 aa, synthetic construct
VMRY

SEQ ID NO: 133 - protein, 8 aa, synthetic construct
  VARIANT 3: note = X is A or I or T
  VARIANT 4: note = X is K or E or G
  VARIANT 7: note = X is L or P or I or V
  VARIANT 8: note = X is A or P
ERXXQAXX

SEQ ID NO: 134 - protein, 19 aa, synthetic construct
  VARIANT 1: note = X is F or I
  VARIANT 2: note = X is L or V
  VARIANT 3: note = X is G or S or F
  VARIANT 4: note = X is I or L or V
  VARIANT 10: note = X is L or M or C
  VARIANT 12: note = X is I or K or M
  VARIANT 13: note = X is A or N or S
  VARIANT 14: note = X is E or N
  VARIANT 15: note = X is I or L
  VARIANT 16: note = X is A or E or S
  VARIANT 17: note = X is R or Q
  VARIANT 18: note = X is I or M
  VARIANT 19: note = X is R or K
  VARIANT 7: note = X is N or E
XXXXAYXTLXRXXXXXXXX

SEQ ID NO: 135 - skipped

SEQ ID NO: 136 - protein, 13 aa, synthetic construct
  VARIANT 9: note = X is R or Q or I or M
  VARIANT 11: note = X is M or L
  VARIANT 12: note = X is R or Q or E or A
  VARIANT 13: note = X is R or Q or E
HSARVGAAXDXXX

SEQ ID NO: 137 - protein, 7 aa, synthetic construct
  VARIANT 3: note = X is A or E
  VARIANT 5: note = X is G or T
  VARIANT 7: note = X is T or S or K or Q
MQXGXWX

SEQ ID NO: 138 - skipped

SEQ ID NO: 139 - DNA, 34 bp, unidentified
  source 1..34: mol_type = other DNA
ataacttcgtatagcatacattatacgaagttat

SEQ ID NO: 140 - protein, 460 aa, unidentified
MDKHHLLSTRTTAPTDGSDFNSDNVTKNHHAILIERALASALARPGLLLETHFQPIYQRADRLGVDLSDCPSFFVATQRLLAQHHQHGAGLSPRSLEQFHSAVRVFTQWCHVNRRTALPASADTFVLFARSNAPHITLSTLHIYVWAIRKLHLITGLVDPTDSQAVKQHLSRIKKQKIAQFDTAQDQAVPLSDEDYRTAMTLLMADDHPISWRDATLLGLAYHTMLRQSELVRIQLTHIQPRSDGDWTLEIPYTKTNKTGRSEYVTLPHYLMPILQRYLSLCGDRTLTDPGYLFVPLTRSGVPRRQRMHVTSTPRAPSGVTPRRKTRGGIQFAPVAPDAVTDSIAAPPDPAPISEEVQPVAPKLVARTLKKIGERLATHTSSPQADRAYSGHSARVGRAIHLLEIGARKEDIVKAGRWKSDIMFERYTRQYDVNDGYLAQIRQAEDRHWHAQTEPPDSTG

(19) United States
(12) Patent Application Publication
(10) Pub. No.: US 2023/0183713 A1
(43) Pub. Date: Jun. 15, 2023

(54) SITE-SPECIFIC RECOMBINASES FOR EFFICIENT AND SPECIFIC GENOME EDITING

(71) Applicant: TECHNISCHE UNIVERSITÄT DRESDEN, Dresden (DE)

(72) Inventors: Jenna HOERSTEN, Dresden (DE); Felix LANSING, Dresden (DE); Frank BUCHHOLZ, Dresden (DE)

(21) Appl. No.: 18/055,545

(22) Filed: Nov. 15, 2022

(30) Foreign Application Priority Data
Nov. 15, 2021 (EP) 21208214.3

(51) Int. Cl. C12N 15/52; C12N 15/63

(52) U.S. Cl. CPC C12N 15/52; C12N 15/63

(57) ABSTRACT

The invention relates to the field of genome editing and provides a method of generating DNA recombinases or provides DNA recombinases, which efficiently and specifically recombine genomic target sequences as obligate DNA recombinase enzymes. The invention provides a genetically engineered DNA recombining enzyme comprising a complex of a first and a second recombinase enzyme, wherein said first recombinase enzyme and said second recombinase enzyme specifically recognize a first half-site and a second half-site of an upstream target site and/or a downstream target site of a DNA recombinase, wherein said first recombinase enzyme and said second recombinase enzyme each comprises at least one mutation in its catalytic site, wherein said first recombinase enzyme and said second recombinase enzyme carrying said at last one mutation in their catalytic site, when expressed in isolation, do not show the catalytic activity of a DNA recombinase, and wherein said first DNA recombinase enzyme and said second DNA recombinase enzyme carrying said at least one mutation in their catalytic site when co-expressed and forming a complex show the catalytic activity of a DNA recombinase. The invention further relates to nucleic acid molecules encoding said genetically engineered DNA recombinases and complexes, as well as to pharmaceutical compositions comprising the same. The invention further provides the use of said genetically engineered DNA recombinases, complexes, nucleic acid molecules and pharmaceutical compositions in medicine and specifically for treating genetic disorders such as hemophilia A.

### RELATED APPLICATIONS

This application claims priority to European Patent Application No. 21208214.3, filed Nov. 15, 2021, the entire disclosure of which is hereby incorporated herein by reference.

### SEQUENCE LISTING

The instant application contains a Sequence Listing which has been submitted electronically in XML format and is hereby incorporated by reference in its entirety. Said XML copy, created Nov. 14, 2022, is named 734829_TUD9-003_ST26.xml, and is 192, 921 bytes in size.

### FIELD OF THE INVENTION

The invention relates generally to the field of genome editing and provides DNA recombinases, which efficiently and specifically recombine genomic target sequences with obligate DNA recombinase enzymes. More specifically, the invention provides genetically engineered DNA recombinases for efficient and specific genome editing, comprising an obligate complex of recombinases, wherein said complex comprises at least a first recombinase enzyme and at least a second recombinase enzyme, wherein said first recombinase enzyme and said second recombinase enzyme specifically recognize a first half-site and a second half-site of an upstream target site and/or a downstream target site of a DNA recognition site of a DNA recombinase; wherein said first recombinase enzyme and said second recombinase enzyme each comprises at least one mutation in a catalytic site, wherein said first recombinase enzyme and said second recombinase enzyme carrying said at last one mutation in a catalytic site, when expressed in isolation, do not show the same catalytic activity of a DNA recombinase; and wherein the catalytic activity as a DNA recombinase in said obligate complex of recombinases is complemented. The invention also discloses obligate complexes of recombinases, which catalyze the recombination of a DNA sequence present in the int1h regions on the human X chromosome. The invention further relates to nucleic acid molecules encoding said genetically engineered DNA recombinases and obligate complexes, as well as to the use of said genetically engineered DNA recombinases and obligate complexes and nucleic acid molecules in genome editing. Moreover, the invention provides a method for generating said obligate DNA recombinases.

### BACKGROUND OF THE INVENTION

Genome engineering is becoming an increasingly important technology in biomedical research. The main approach in the field of gene editing nowadays is the nuclease-mediated introduction of double-strand breaks (DSB) at the locus of interest that are subsequently corrected by the cellular repair pathways. There are four types of programmable nucleases that can be divided into two groups based on their mode of target DNA sequence recognition. Meganucleases, zinc finger nucleases (ZFNs) and transcription activator-like nucleases (TALENs) guide the nuclease to the specific locus using protein-DNA interactions, while clustered, regularly interspaced, short-palindromic repeat-associated (CRISPR) endonucleases direct it using RNA-DNA interactions (34, 35). Programmable nucleases are candidates for therapeutic application, and several of them are already in clinical trials (36, 37, 38).

Yet, one of the main challenges of programmable nucleases is the risk of unpredictable sequence rearrangements. The introduced DSBs are repaired by the cells primarily using non-homologous end-joining (NHEJ) or homology-directed repair (HDR). The repair by HDR is precise and maintains genomic stability, as the sequence is copied from the second allele or a donor sequence that matches the target. However, HDR is mainly active during DNA replication, and in most cells NHEJ events outnumber HDR. NHEJ is an error-prone repair mechanism, which leads to insertions and deletions (indels) in the repaired DNA fragment. This may result in adverse events due to alteration of gene sequence (34; 39, 40).

Alternative tools that are widely used for genome engineering include site-specific recombinases (SSRs) from the tyrosine recombinase family. Tyrosine SSRs have considerable advantages over programmable nucleases, as they are not dependent on the cellular DNA repair pathways because they perform the full recombination reaction without any accessory factors (2). This leads to highly specific, predictable and precise genome editing events, which makes them attractive for therapeutic applications.

One of the most commonly used SSRs is Cre, a tyrosine site-specific recombinase (SSR), which forms a homotetramer that stringently catalyzes recombination of DNA between loxP target sites (1). The loxP sequence is composed of two 13 bp palindromic half-sites flanking an 8 bp spacer region where recombination occurs (FIG. 1A). The Cre/loxP system is commonly used for genomic modifications because of its precision and robustness in orthologous hosts (2). However, application of Cre/loxP across organisms requires pre-introduction of loxP target sequences into the host genome, which can be time-consuming and cumbersome (3, 4). To overcome this limitation and to broaden the use of SSRs, directed molecular evolution has been utilized to produce Cre variants with altered DNA specificities for predefined (pseudo)-symmetric DNA target sites naturally occurring within host genomes (5-10). The practicality of this approach was first demonstrated where Tre, an evolved Cre-type enzyme, was generated to specifically recognize and excise HIV-1 proviral DNA from the human genome (7). Tre and other evolved SSRs represent the potential for site-specific recombinases to be adapted for a broad range of therapeutic applications (8, 11, 12).

Significant progress has been made to engineer novel SSRs capable of recombination on a range of DNA substrates (13-45), including non--symmetric sites (5, 9, 10). The recombination of non-symmetric target sites was first demonstrated by co-expression of wild-type Cre and mutant Cre molecules that together catalyzed recombination between artificial asymmetric loxP-loxM7 sites, demonstrating proof of concept that engineered heterospecific SSRs can be generated (5). This general principle has recently been extended to achieve recombination between asymmetric target sequences naturally occurring in the human genome by combining two evolved Cre variants (9). The Cre-type molecules, each with unique haft-site specificities, were first generated through directed evolution on their respective symmetric sites. The distinct variants were then expressed together forming a functional heterotetramer capable of specifically excising a DNA fragment flanked by the desired asymmetric human target sites (9). More recently, this approach has been demonstrated to be applicable to correct a chromosomal inversion causing a genetic human disorder (10). By combining two designer-recombinases targeting the asymmetric loxF8 sequence located on the human X-chromosome, Lansing et al. showed that the heterotetramer (D7) could efficiently correct the genomic int1h inversion to reestablish factor VIII expression in patient-derived cells (10). The D7 SSR is composed of two unique Cre-type subunits, D7L and D7R, each evolved to bind to their corresponding half site, loxF8L and loxF8R, respectively.

However, using more than one recombinase with different target specificities inherently carries risks. By using several Cre-derived recombinases with different specificities, there is an increased possibility of subunit assembly into undesired functional complexes, including homotetramers that could cause recombination events at non-target sites. To mitigate these potential off-target effects, approaches to assure only the formation of heterotetramers are critical to increase their safety in therapeutic applications (10). Previously, prevention of homotetramer formation was achieved through structure-guided redesign of several residues implicated in the protein-protein interaction interface between the different recombinase monomers (16). Hence, this approach to generate obligate SSR systems is limited to enzymes with available crystal structures and is therefore not easily adaptable to engineered or distantly related recombinases.

WO 2021./110846 discloses fusion proteins for efficient and specific genome editing, comprising a complex of recombinases comprising at least a first recombinase enzyme and a second recombinase enzyme, wherein said first recombinase enzyme and said second recombinase enzyme are interconnected via an oligopeptide linker. The efficiency of these fusion proteins depends on the properties of the linker.

U.S. Pat. No. 10, 017, 832 B2 discloses DNA recombinases that have been produced by introducing several mutations in the protein-protein interface between the DNA recombinase monomers to form so called obligate heterotetrameric complexes. This approach, however, has the disadvantage that it requires extensive efforts to introduce several mutations into each of the recombinase enzymes, which also leads to a decrease of their recombinase activity compared to the wild type enzymes not comprising the mutations, for allowing formation of the obligate heterotetrameric complex. In addition, because the targeted residues are not well conserved in related recombinases, it is not straight forward to make obligate versions of other naturally occurring enzymes.

### SUMMARY OF THE INVENTION

It is therefore the objective problem of the present invention to overcome the disadvantages of the prior art and to provide a complex of recombinases having improved properties, in particular a catalytic activity that allows efficient recombination events, increased specificity and/or diminished activity at non-target or off-target sites.

This problem is solved by the provision of genetically engineered DNA recombinases for efficient and specific genome editing, comprising an obligate complex of recombinases, wherein said complex comprises at least a first recombinase enzyme and at least a second recombinase enzyme, wherein said first recombinase enzyme and said second recombinase enzyme specifically recognize a first half-site and a second half-site of an upstream target site and/or a downstream target site of a DNA recombinase; wherein said first recombinase enzyme and said second recombinase enzyme each comprises at least one mutation in their catalytic region; wherein said first recombinase enzyme and said second recombinase enzyme carrying said at last one mutation, when expressed in isolation, do not show the catalytic activity of a DNA recombinase; and wherein the catalytic activity as a DNA recombinase in said obligate complex of recombinases is complemented.

The present invention is not based on the redesign of the protein-protein interface between DNA recombinase monomers, as disclosed e.g. in U.S. Pat. No. 10, 017, 832 B2. In contrast to the prior art, said at least one mutation in the catalytic region of the first recombinase enzyme and in the catalytic region of the second recombinase enzyme are not present in the region that is responsible for the protein-protein-interaction between the recombinase enzymes or in the DNA binding regions, thereby avoiding any undesired interference with protein-protein complex formation and with target recognition. Advantageously, the recombinase complex of the present invention is only active on the desired target sites when the correct subunits come together, mutually complement their catalytic activity and thereby form an active complex.

Preferably, said at least one mutation is a point mutation in form of an amino acid substitution, which leads to the replacement of an amino acid in the protein sequence of the first recombinase enzyme and to the replacement of an amino acid in the amino acid sequence of the second recombinase enzyme. Most preferably, the at least one point mutation is a single amino acid substitution in the catalytic region of the respective recombinase. According to a preferred embodiment, the first recombinase enzyme and the second recombinase enzyme do not have a point mutation at the same position or of the same type. Not having a point mutation of the same type means that if the mutation is at the same position in the first and the second recombinase, a first amino acid that is substituted for the amino acid at this position in the first recombinase is different from a second amino acid that is substituted for the amino acid at this position in the second recombinase.

Tyrosine site-specific recombinases represent a versatile genome editing tool with considerable therapeutic potential. Recent developments to engineer and evolve SSRs into heterotetramers to improve target site flexibility signified a critical step towards their broad utility in genome editing. However, monomers of tyrosine site-specific recombinases tend to form combinations of different homo- and heterotetramers in cells, increasing their off-target potential. Therefore, the present invention provides two paired mutations targeting residues implicated in catalysis, leading to simple obligate systems of tyrosine site-specific recombinases. Only when the paired mutations are applied as single mutations on each recombinase subunit (e.g. first and second recombinase enzyme), the engineered tyrosine site-specific recombinases can efficiently recombine the intended target sequence, while the monomers carrying the point mutations expressed in isolation are inactive. The utility of the obligate system of tyrosine site-specific recombinases to improve recombination specificity of a designer-recombinase for a therapeutic target in human cells is demonstrated herein. Furthermore, it is shown that the mutations render certain naturally occurring tyrosine site-specific recombinases, such as Cre, Vika, Panto, Dre, etc. obligate, providing a straight-forward approach to improve their applied properties. The present invention contributes to the development of safe and effective therapeutic designer-recombinases and advance the mechanistic understanding of the catalysis by tyrosine site-specific recombinases. Undesired side reactions, i.e. off-target recombination events can thereby be effectively avoided. The target specificity of the genetically engineered DNA recombinases is further increased compared to approaches known in the prior art.

A disadvantage of monomer—monomer interface mutations (as disclosed e.g. in U.S. Pat. No. 10, 017, 832 B2) is the reduced recombination activity of the engineered recombinases compared to the wild-type enzymes. In contrast thereto, the engineered recombinases of the present invention, which comprise at least a single mutation in a catalytic site, do not show such a loss of recombination activity. It is demonstrated herein that the recombination activity of the engineered recombinases of the present invention is comparable to the recombination activity of the wild-type enzymes, accompanied by a drastically improved target specificity as aforementioned.

According to one aspect, the present invention provides a method for generating obligate DNA recombinases for genome editing, wherein said method comprises the steps of:

i. providing a nucleic acid molecule encoding a first recombinase enzyme and a nucleic acid molecule encoding a second recombinase enzyme, wherein said first recombinase enzyme binds to a first half site of an asymmetric recombinase target site and said second recombinase enzyme binds to a second half site of an asymmetric recombinase target site, wherein said first recombinase enzyme and said second recombinase enzyme form a heterodimer, which is capable to induce a site-specific DNA recombination of a sequence of interest at said asymmetric recombinase target site in a DNA sequence, wherein said asymmetric recombinase target site comprises a first half site and a second half site of an upstream target site and/or a downstream target site of a DNA recombinase wherein said first half site and a second half site are not identical and which are not palindromic;

ii. mutagenesis to create libraries of nucleic acid molecules encoding mutant first recombinase enzymes and of nucleic acid molecules encoding mutant second recombinase enzymes, wherein mutations are introduced in said first recombinase enzyme and said second recombinase enzyme;

iii. creating expression vectors, by cloning the library of the nucleic acid molecules encoding a first mutant recombinase enzyme and the library of nucleic acid molecules encoding a second mutant recombinase enzyme into expression vectors, wherein said expression vectors carry a DNA sequence of interest, which is to be recombined;

iv. transfecting a cell with the expression vectors of step iii) and expressing the libraries of said mutant first recombinase enzyme and said mutant second recombinase enzyme in the same cell resulting in the formation of recombinase heterodimers comprising a mutant first recombinase enzyme and a mutant second recombinase enzyme;

v. positive selection screen for heterodimers obtained in step iv. that are capable to induce a site-specific DNA recombination of a sequence of interest at an asymmetric recombinase target site in a DNA;

vi. negative selection screen for heterodimers obtained in step iv. or v. that are not capable to induce a site-specific DNA recombination of a sequence of interest at an off-target, preferably symmetric recombinase target site in a DNA; and

vii. selecting an obligate DNA recombinase which is capable of recombining a DNA sequence of interest at a recombinase target site in a DNA comprising a first half site and a second half site of an upstream target site and/or a downstream target site of a DNA recombinase, wherein said first half site and said second half site are not identical and are not palindromic; and which is not capable of recombining a DNA sequence of interest at an off-target, preferably symmetric recombinase target site in a DNA;

wherein in said obligate DNA recombinase obtained in step vii., said first mutant recombinase enzyme and said second mutant recombinase enzyme each comprise at least one mutation in a catalytic site, which render said first recombinase enzyme and said second recombinase enzyme catalytically inactive when expressed in isolation.

According to a preferred embodiment, the obligate DNA recombinases are for recombination of DNA sequences.

According to one embodiment, the first recombinase enzyme and the second recombinase enzyme according to steps ii. to vi. are evolved by substrate linked directed evolution (SLiDE) or directed evolution.

According to a further embodiment, the selection according to steps v. and vi. iterates between the selection for obligate heterodimers that are catalytically active on the asymmetric target sites (positive selection), and between heterodimers that are not catalytically active on off-target sites, preferably symmetric target sites.

According to a further aspect, the present invention provides a genetically engineered DNA recombining enzyme for genome editing, wherein said DNA recombining enzyme comprises an obligate complex of recombinases, wherein said complex comprises at least a first recombinase enzyme and at least a second recombinase enzyme, wherein said first recombinase enzyme and said second recombinase enzyme specifically recognize a first half-site and a second half-site of an upstream target site and/or a downstream target site of a DNA recombinase, wherein said first recombinase enzyme and said second recombinase enzyme each comprises at least one mutation in a catalytic site; wherein said first recombinase enzyme and said second recombinase enzyme carrying said at last one mutation in a catalytic site, when expressed in isolation, do not show the catalytic activity of a DNA recombinase; and wherein the catalytic activity as a DNA recombinase in said obligate complex of recombinases is complemented.

According to a preferred embodiment, said first half site and said second half site are not identical and are not palindromic.

According to a further preferred embodiment, the genetically engineered DNA recombining enzyme is obtained by the method for generating obligate DNA recombinases for genome editing according to the present invention.

According to one embodiment, the at least one first recombinase and the at least one second recombinase are of the same type. Preferably, the at least one first recombinase and the at least one second recombinase are both Cre-, Dre-, VCre-, SCre-, Vika-, lambda-Int-, Flp-, R-, Kw-, Kd-, B2-, B3-, Nigri- or Panto-recombinases.

According to a further embodiment, the DNA recombining enzyme is a complex of recombinases in form of a heterotetramer.

According to another embodiment, the at least one mutation is a single amino acid substitution in the catalytic region of the recombinase. Preferably, said single amino acid substitution in the at least one first and in the at least one second recombinase enzyme is at a position of a conserved amino acid in the catalytic region.

According to a preferred embodiment, the present invention provides a genetically engineered DNA recombining enzyme comprising a complex of at least a first DNA recombinase enzyme and at least a second DNA recombinase enzyme, wherein said first DNA recombinase enzyme and said second DNA recombinase enzyme specifically recognize a first half-site and a second half-site of an upstream target site and/or a downstream target site of a DNA recombinase, wherein said first DNA recombinase enzyme and said second DNA recombinase enzyme each comprises a single amino acid substitution in their catalytic region, wherein said first DNA recombinase enzyme and said second DNA recombinase enzyme carrying said single amino acid substitution when expressed in isolation do not show the catalytic activity of a DNA recombinase, and wherein said first DNA recombinase enzyme and said second DNA recombinase enzyme carrying said single amino acid substitution when co-expressed and forming a complex show the catalytic activity of a DNA recombinase.

According to one embodiment, the at least one mutation or the single mutation in the catalytic region of the first DNA recombinase is different from the least one mutation or the single mutation in the catalytic region of the second DNA recombinase.

According to another embodiment, the present invention provides a DNA recombining enzyme of the invention, wherein
(i) the first recombinase enzyme comprises a Cre recombinase and the second recombinase enzyme comprises a Cre recombinase, wherein each Cre recombinase comprises a single amino acid substitution within an amino acid region selected from the group consisting of SEQ ID NO: 109 to SEQ ID NO: 114;
(ii) the first recombinase enzyme comprises a Vika recombinase and the second recombinase enzyme comprises a Vika recombinase, wherein each Vika recombinase comprises a single amino acid substitution within an amino acid region selected from the group consisting of SEQ ID NO: 115 to SEQ ID NO: 120;
(iii) the first recombinase enzyme comprises a Dre recombinase and the second recombinase enzyme comprises a Dre recombinase, wherein each Dre recombinase comprises a single amino acid substitution within an amino acid region selected from the group consisting of SEQ ID NO: 121 to SEQ ID NO: 126; or
(iv) the first recombinase enzyme comprises a Panto recombinase and the second recombinase enzyme comprises a Panto recombinase, wherein each Panto recombinase comprises a single amino acid substitution within an amino acid region selected from the group consisting of SEQ ID NO: 127 to SEQ ID NO: 132.

According to one embodiment, said first recombinase enzyme and said second recombinase enzyme do not comprise monomer-monomer-interface mutations.

According to another embodiment, said genetically engineered DNA recombining enzyme is a mutant of a naturally occurring site-specific recombinase or a mutant of designer DNA recombinase.

According to a preferred embodiment, the at least one mutation or the single amino acid substitution in the at least one first and in the at least one second recombinase enzyme is selected from the group consisting of: E129R, Q133H, R173A, R173C, R173D, R173E, R173F, R173G, R173I, R173K, R173L, R173M, R173N, R173P, R173Q, R173S, R173T, R173V, R173W, R173Y, E176H, E176I, E176L, E176M, E176V, E176W, E176Y, K201A, K201C, K201C, K201D, K201F, K201G, K201H, K201I, K201L, K201M, K201N, K201P, K201Q, K201R, K201S, K201T, K201V, K201W, K201Y, H289D, H289E, H289I, H289K, H289R, H289W, R292A, R292C, R292E, R292F, R292G, R292H, R292I, R292L, R292M, R292N, R292P, R292Q, R292S, R292T, R292V, R292W, R292Y, Q311R, W315C, W315E, W315G, W315I, W315K, W315L, W315M, W315N, W315Q, W315R, W315S, W315T, W315V, Y324A, Y324C, Y324E, Y324F, Y324H, Y324I, Y324K, Y324L, Y324M, Y324N, Y324Q, Y324R, Y324S, Y324T, Y324V, and Y324W of SEQ ID NO: 1, or in a corresponding amino acid position of another recombinase, preferably in a corresponding position of SEQ ID NO: 14, SEQ ID NO: 17 or SEQ ID NO: 20.

According to a further preferred embodiment, the at least one mutation or the single amino acid substitution in the at least one first and in the at least one second recombinase enzyme is at a position selected from the group consisting of:

(i) E129, Q133, R173, E176, K201, H289, R292, Q311, W315, and Y324 of SEQ ID NO: 1;

(ii) E146, Q151, R191, N194, K219, H308, R311, Q330, W334, and Y343 of SEQ ID NO: 14;

(iii) E130, Q134, R174, E177, K202, H290, R293, Q312, W316, and Y325 of SEQ ID NO: 17;

(iv) E131, Q135, R175, E178, K202, H290, R293, Q312, W316, and Y325 of SEQ ID NO: 20; or

(v) at an amino acid position in another recombinase, wherein said amino acid position in the other recombinase corresponds to position E129, Q133, R173, E176, K201, H289, R292, Q311, W315, or Y324 of SEQ ID NO: 1.

According to one embodiment, said first recombinase enzyme and said second recombinase enzyme do not comprise monomer-monomer-interface mutations.

According to another embodiment, said genetically engineered DNA recombining enzyme is a mutant of a naturally occurring site-specific recombinase or a mutant of designer DNA recombinase.

According to a preferred embodiment, said first recombinase enzyme comprises the mutation selected from the group consisting of mutation K201R of SEQ ID NO: 1, 2 or 7; K202R of SEQ ID NO: 18 or 21, mutation K219R of SEQ ID NO: 15, and mutation K221R of SEQ ID NO: 24; and said second recombinase enzyme comprises the mutation selected from the group consisting of mutation Q311K of SEQ ID NO: 4 or 11, mutation Q311R of SEQ ID NO: 5 or 12, mutation Q312R of SEQ ID NO: 19 or 22, mutation Q330R of SEQ ID NO: 16, and mutation Q336R of SEQ ID NO: 25.

According to one embodiment, said recombinase target site is a target site of a tyrosine site-specific recombinase such as Cre-, Dre-, VCre-, SCre-, Vika-, lambda-Int-, Flp-, R-, Kw-, Kd-, B2-, B3-, Nigri- and Panto-recombinase, and said genetically engineered DNA recombining enzyme is selected from the group consisting of:

a genetically engineered DNA recombining enzyme, comprising a first recombinase enzyme and a second recombinase enzyme, wherein the first recombinase enzyme is a polypeptide which has an amino acid sequence with at least 70%, preferably 80%, more preferably 90% sequence identity with a sequence according to SEQ ID NO: 7 and comprises the single mutation K201R in the catalytic site; and wherein the second recombinase enzyme is a polypeptide which has an amino acid sequence with at least 70%, preferably 80%, more preferably 90% sequence identity with a sequence according to SEQ ID NO: 11 and comprises the single mutation at position Q311R in the catalytic site;

a genetically engineered DNA recombining enzyme, comprising a first recombinase enzyme and a second recombinase enzyme, wherein the first recombinase enzyme is a polypeptide which has an amino acid sequence with at least 70%, preferably 80%, more preferably 90% sequence identity with a sequence according to SEQ ID NO: 7 and comprises the single mutation K201R in the catalytic site; and wherein the second recombinase enzyme is a polypeptide which has an amino acid sequence with at least 70%, preferably 80%, more preferably 90% sequence identity with a sequence according to SEQ ID NO: 12 and comprises the single mutation Q311K in the catalytic site;

Cre recombinase, comprising a first recombinase enzyme having the single mutation K201R in its catalytic site and having an amino acid sequence which is at least 70%, preferably 80%, more preferably 90% identical with a polypeptide having the sequence according to SEQ ID NO: 2; and comprising a second recombinase enzyme having the single mutation Q311R in its catalytic site and having an amino acid sequence which is at least 70%, preferably 80%, more preferably 90% identical with a polypeptide having the sequence according to SEQ ID NO: 5;

Cre recombinase, comprising a first recombinase enzyme and having the single mutation K201R in its catalytic site and having an amino acid sequence which is at least 70%, preferably 80%, more preferably 90% identical with a polypeptide having the sequence according to SEQ ID NO: 2; and comprising a second recombinase enzyme having the single mutation Q311K in its catalytic site and having an amino acid sequence which is at least 70%, preferably 80%, more preferably 90% identical with a polypeptide having the sequence according to SEQ ID NO: 4;

Vika recombinase, comprising a first recombinase enzyme having the single mutation K219R in its catalytic site and having an amino acid sequence which is at least 70%, preferably 80%, more preferably 90% identical with a polypeptide having the sequence according to SEQ ID NO: 15; and a second recombinase enzyme having the single mutation Q330R in its catalytic site and having an amino acid sequence which is at least 70%, preferably 80%, more preferably 90% identical with a polypeptide having the sequence according to SEQ ID NO: 16;

Panto recombinase, comprising a first recombinase enzyme having the single mutation K202R in its catalytic site and having an amino acid sequence which is at least 70%, preferably 80%, more preferably 90% identical with a polypeptide having the sequence according to SEQ ID NO: 18; and a second recombinase enzyme having the single mutation Q312R in its catalytic site and having an amino acid sequence which is at least 70%, preferably 80%, more preferably 90% identical with a polypeptide having the sequence according to SEQ ID NO: 19;

Dre recombinase, comprising a first recombinase enzyme having the single mutation K202R in its catalytic site and having an amino acid sequence which is at least 70%, preferably 80%, more preferably 90% identical with a polypeptide having the sequence according to SEQ ID NO: 21; and a second recombinase enzyme having the single mutation Q312R in its catalytic site and having an amino acid sequence which is at least 70%, preferably 80%, more preferably 90% identical with a polypeptide having the sequence according to SEQ ID NO: 22; and

Vcre recombinase, comprising a first recombinase enzyme having the single mutation K221R in its catalytic site and having an amino acid sequence which is at least 70%, preferably 80%, more preferably 90% identical with a polypeptide having the sequence according to SEQ ID NO: 24; and a second recombinase enzyme having the single mutation Q336R in its catalytic site and having an amino acid sequence which is at least 70%, preferably 80%, more preferably 90% identical with a polypeptide having the sequence according to SEQ ID NO.: 25.

According to another embodiment, said genetically engineered DNA recombining enzyme specifically recognizes the upstream recombinase target sequence of the loxF8 target site, which has the nucleic acid sequence ATAAATCTGTGGAAACGCTGCCACACAATCTTAG (SEQ ID NO: 65) or a reverse complement sequence thereof; and recognizes the downstream recombinase target sequence of the loxF8 target site, which has the nucleic acid sequence CTAAGATTGTGTGGCAGCGTTTCCACAGATTTAT (SEQ ID NO: 66) or a reverse complement sequence thereof; and which catalyzes the recombination of a gene sequence between the upstream recombinase target site sequence of SEQ ID NO: 65 and the downstream recombinase target site sequence of SEQ ID NO: 66 of the loxF8 recombinase target site; and wherein the capability to catalyze the recombination of a gene sequence between the upstream recombinase target sequence of SEQ ID NO: 65 and the downstream recombinase target sequence of SEQ ID NO: 66 of the loxF8 recombinase target site is tested by a method comprising the steps of:
a) expressing the genetically engineered DNA recombining enzyme comprising a first recombinase enzyme and a second recombinase enzyme in a cell; and
b) analyzing, whether the genetically engineered DNA recombining enzyme expressed in step a) is capable of recombining of a DNA sequence on a human chromosome in said cell.

According to a further aspect, the present invention provides a nucleic acid molecule or a plurality of nucleic acid molecules each comprising or consisting of a nucleic acid sequence encoding a genetically engineered DNA recombining enzyme or a subunit thereof according to the present invention.

According to a yet another aspect, the present invention provides an expression vector comprising the nucleic acid molecule or the plurality of nucleic acid molecules according to the invention and expression-controlling elements operably linked with said nucleic acid to drive expression thereof.

According to a further aspect, the present invention provides a mammalian, insect, plant or bacterial host cell comprising the nucleic acid molecule or the plurality of nucleic acid molecules according to the invention or the expression vector comprising the nucleic acid molecule according to the invention.

According to a yet another aspect, the present invention provides a pharmaceutical composition comprising the genetically engineered DNA recombining enzyme according to the invention or the nucleic acid molecule or the plurality of nucleic acid molecules of the invention, or the expression vector of the invention, or the cell of the invention. Optionally, the pharmaceutical composition further comprises one or more therapeutically acceptable diluents or carriers.

According to a further aspect, the present invention provides the genetically engineered DNA recombining enzyme according to the invention, or the nucleic acid molecule or the plurality of nucleic acid molecules according to the invention, or the expression vector according to the invention, or the pharmaceutical composition according to the invention, for use in medicine.

According to a yet another aspect, the present invention provides the genetically engineered DNA recombining enzyme according to the invention, or the nucleic acid molecule or the plurality of nucleic acid molecules according to the invention, or the expression vector according to the invention, or the pharmaceutical composition according to the invention, for use in the treatment of hemophilia A. According to a preferred embodiment, the treatment is treatment of severe hemophilia A.

According to a further aspect, the present invention provides a method for inversion of a DNA sequence on genomic level in a cell in vitro, comprising a genetically engineered DNA recombining enzyme according to the invention, wherein said method comprises the steps of:

i. providing a nucleic acid molecule encoding a first recombinase enzyme of the present invention, wherein said first recombinase enzyme comprises at least one mutation that inactivates the catalytic activity as a DNA recombinase of said first recombinase enzyme and wherein said first recombinase monomer specifically recognizes a first half-site of a recombinase target site;

ii. providing a nucleic acid molecule encoding a second recombinase enzyme of the present invention, wherein said second recombinase enzyme comprises at least one mutation that inactivates the catalytic activity as a DNA recombinase of said second recombinase enzyme and wherein said second recombinase monomer specifically recognizes a second half-site of a recombinase target site;

iii. creating an expression vector by cloning the nucleic acid molecule encoding a first recombinase enzyme and the nucleic acid molecule encoding a second recombinase enzyme into an expression vector;

iv. delivering said expression vector to a cell, which comprises a DNA sequence, which is to be inverted, an expression vector of step iii), or a RNA molecule encoding a genetically engineered DNA recombining enzyme of the present invention;

v. expressing a genetically engineered DNA recombining enzyme of the present invention;

vi. inversion of a DNA sequence, which is to be inverted, on a human chromosome in said cell with said genetically engineered DNA recombining enzyme of the present invention expressed in said cell.

According to a preferred embodiment, said cell is not a human germ cell.

According to a further aspect, the present invention provides a method for treating or preventing a disease, comprising administering to a subject in need thereof the genetically engineered DNA recombining enzyme according to the present invention, or the nucleic acid molecule or the plurality of nucleic acid molecules according to the present invention, or the expression vector according to the present invention, or the host cell according to the present invention, or the pharmaceutical composition according to the present invention.

According to a further aspect, the present invention provides a method for treating or preventing hemophilia A, comprising administering to a subject in need thereof the genetically engineered DNA recombining enzyme according to the present invention, or the nucleic acid molecule or the plurality of nucleic acid molecules acc according to the present invention, or the expression vector according to the present invention, or the host cell according to the present invention, or the pharmaceutical composition according to the present invention, optionally wherein the hemophilia A is severe hemophilia A.

According to a further aspect, the present invention provides a method for recombination of a target DNA sequence in a cell, comprising introducing into the cell:

(a) a nucleic acid molecule encoding the first recombinase enzyme and a nucleic acid molecule encoding the second recombinase enzyme according to the present invention; and/or

(b) the first recombinase enzyme and the second recombinase enzyme according to the present invention,

thereby recombining the target DNA sequence in the cell.

According to a preferred embodiment, the nucleic acid molecule encoding the first recombinase enzyme and/or the nucleic acid molecule encoding the second recombinase enzyme is an mRNA.

According to an embodiment, the nucleic acid molecule encoding the first recombinase enzyme and/or the nucleic acid molecule encoding the second recombinase enzyme is an expression vector.

Further aspects and embodiments of the present invention will become apparent from the accompanying claims and the following detailed description.

### BRIEF DESCRIPTION OF THE DRAWINGS

FIG. 1 shows the protein-protein interface alteration of the heterospecific D7 recombinase. (A) Nucleotide sequences of relevant SSR target sites. loxF8, loxF8L and loxF8R represent target sequences for the D7L and D7R recombinases, respectively. Highlight are some features (bold—differences to the loxP site, underlined—asymmetric residues of loxF8 sites) (B) Bacterial assay showing activity of co-expression of D7L and D7R (recombinase enzyme) on target sites loxF8, loxF8L and loxF8R (substrate). Recombination is signified by the band aligned with the single triangle and non-recombined events are indicated by two triangles. The concentration of L-Arabinose (ug/mL) used for induction of recombinase expression is shown on the bottom. (C) Schematic representation of the desired obligate D7 recombinase activity. D7LA3 and D7RB2 harbor mutations that should only allow heterodimer formation on the loxF8 target site. The mutations should prevent D7LA3 and D7RB2 homodimer formation on the symmetric loxF8R and loxF8L sites. (D) Bacterial assay of recombination activity of co-expression of D7LA3 and D7RB2 on indicated target sites. The concentration of L-Arabinose (ug/mL) used for induction of recombinase expression is shown on the bottom.

FIG. 2 shows the selection method, comparing the activity of the starting library and the activity of the end library and subsequent analysis of acquired mutations. (A) Selection scheme where positive selection pressure was placed on the library to select recombinases active on the loxF8 site and negative selection pressure to select for the variants that are not active on the symmetric loxF8L or loxF8R sites (indicated by an X). (B) Bacterial assay indicating activity of start library (targeted library generated by applying ISOR method to the D7 recombinase sequence) and end library (library after 26 rounds of evolution and selection pressure) on target sites (Substrate) loxF8, loxF8L and loxF8R. The concentration of L-Arabinose (μg/mL) used for induction of recombinase expression is shown on the bottom. (C) Mutation frequency calculated as the amount of times the amino acid position was mutated compared to the D7 sequence divided by the total amount of sequences. The amino acid position for D7L and D7R is plotted along the x-axis and the mutation frequency as a percent of all sequences is plotted along the y-axis. Positions with a mutation frequency over 20% are labeled. Plotted positions for D7R begin at the 6th amino acid position due to high background of sequencing reads. (D) Activity of the isolated D7 monomers on asymmetric site loxF8 and symmetric sites loxF8L and loxF8R. (E) Activity of D7 compared to heterodimer D7LK201R+D7RQ311R on asymmetric site loxF8 and symmetric sites loxF8L and loxF8R. Induction level of L-Arabinose (ug/ml) listed along the bottom.

FIG. 3 shows the obligate D7 activity in mammalian cells. (A) Schematic presentation of mRNA expression constructs and reporter HEK293T cell line. Co-transfection of HEK293T cells with mRNAs expressing an NLS with the recombinase, or BFP. Schematic presentation of loxF8 integrated reporter construct in HEK293T cells. loxF8 sites flank a puromycin selection gene. After successful recombination (excision), mCherry is expressed from the SFFV promoter. (B) Representative brightfield and mCherry fluorescent images of reporter HEK293T cells transfected with indicated recombinases. The 100 “m scale bar is indicated. (C) Recombination efficiency analyzed by FACS and displayed by percent of cells expressing mCherry of the total cells containing BFP. *Normalized to BFP. (D) Scheme of genomic inversion detection PCR. Two PCR programs ran in parallel on extracted genomic DNA of HEK293T cells 48 hours post transfection of recombinase mRNA. The WT orientation is indicated by the amplicon produced by primers. Inverted orientation is also indicated by primers. (E) Resulting PCR with untreated cells across the top and cells transfected with mRNA in the bottom row. Controls consist of HEK293T cells that are transfected with BFP and not recombinase mRNA (non-treated), patient iPSCs carrying the inversion of exon1 (inverted-Ctrl), non-inverted amplicon (WT-Ctrl) and water. (F) Sequences of predicted off-target sites in the human genome with high sequence similarity to loxF8L (HG1L and HG2L) and loxF8R (HE1R and HG2R). (G) Recombination activity of D7 compared to the activity of D7K201R+D7RQ311R on the predicted human off-target sites.

FIG. 4 shows the broad application of obligate mutations to additional recombinase systems. (A) Recombination activity in bacteria of Cre compared to Cre with the obligate mutations expressed as a heterodimer composed of both mutated subunits, CreK201R and CreQ311R on loxP, or expressed as a single mutated subunit of CreK201R or CreQ311R in isolation on loxP. (B) Recombination activity in bacteria of Vika compared to Vika with the obligate correlating mutations of K291R in one subunit and Q330R in the second mutated subunit. First both Vika mutants are expressed together on vox or expressed as a mutated subunit of VikaK219R or VikaQ33OR in isolation on vox. (C) mRNA expression construct and HEK293T loxP reporter cell line. (D) HEK293T bright field and fluorescent mCherry cell images. (E) Recombination efficiency in HEK293T cells of Cre/loxP and combinations of obligate Cre subunites. CreK201R and CreQ311R together and then the subunit CreK201R and subunit Q311R expressed in isolation of the other mutant. Recombination percent normalized to BFP.

FIG. 5 shows structural details of Cre and the studied mutants. (A) MD-refined structure of the Cre complex (PDBID 3C29). One DNA loxP molecule is shown in complex with two Cre monomers (active (A) and inactive (I), respectively). Residues K201 and Q311 are depicted as spheres. (B) LoxP sequence with top strand (TS, SEQ ID NO: 51) and bottom strand (BS, SEQ ID NO: 139) labelled. Arrows indicate attack points by the catalytic tyrosine (Y324) in the respective DNA strands, the first cut indicated by the upper arrow. Bases in the spacer region are labelled. (C)-(F) Superimposition of the MD-refined structures (taken from last 50 ns of simulation) of Cre and mutants Cre_{K201R(A)-K201R(I)}/loxP (C), Cre_{Q311R(A)-Q311R(I)}/loxP (D), Cre_{K201R(A)-Q311R(I)}/loxP (E) and Cre_{Q311R(A)-K201R(I)}/loxP (F). In the mutant structures, the active (A) and inactive (I) monomers are depicted at the left and right hand sides of the figures, respectively, and the DNA is indicated. Side chains of relevant residues are shown in balls and sticks and numbered. Intermolecular H-bonds are depicted with dashed lines. The lack of H-bonds between 1289 in the active monomer of Cre_{Q311R(A)-Q311R(I)} and the phosphate group of A4′_{TS} in loxP is highlighted with two arrows pointing at those atoms (panel D). For clarity, cartoon representations are shown with transparency. The tick and cross symbols indicate active versus inactive mutants. (G) Results of recombination testing of dimers comprising monomers with mutations K201 and Q311.

FIG. 6 shows a plasmid-based assay to visualize recombination activity. The vector carrying the recombinase or recombinase library of interest and the target sites. Upon recombination a 750 bp fragment flanked by the lox-sites is excised. Protein coding genes are depicted as arrows, the origin of replication (oriP15A) and the pBAD promoter as rectangles and the target site as triangles. The protein coding genes include the chloramphenicol resistance gene (cmR), the arabinose regulatory protein (araC) and the genes encoding for the recombinase(s) or libraries of interest. The restriction enzyme sites are shown as dotted lines. Ndel and Avrll are used for selecting vectors that have been recombined. Sacl and Sbfl are used to visualize if plasmid is non-recombined or recombined. The enzymes excise the 2.2 KB fragment containing the recombinase and either a 5 KB or 4.2 KB fragment if the plasmid is non-recombined or recombined, respectively.

FIG. 7 shows the selection strategy. (A) Modified positive selection scheme for substrate-linked directed evolution. The variants that are not active on the loxF8 target site are removed from the library by digesting the purified plasmid with Ndel/Avrll linearizing any plasmid that has not undergone recombination. The active variants are then amplified with primers F and R1 and error-prone Tag Polymerase to add mutations and carry the variants to the next cycle of evolution. (B) Modified negative selection scheme to remove variants capable of recombining the symmetric sites of loxF8L or loxF8R. Here only loxF8L site is depicted due to the selection method remaining the same for both sites. The variants are amplified with primer F and R2 that binds between the symmetric sites, amplifying only those that are not recombined. Tag polymerase is used to add mutations and the variants are carried on to the next cycle of evolution.

FIG. 8 shows a blue-white screen of the library and activity test of a sample of selected colonies. (A) The selection plasmid contains transcriptional terminators flanked by the symmetric sites upstream of LacZa. Upon the symmetric site recombination, the transcriptional terminators are excised allowing for the transcription of LacZa driven by the constitutive cat promoter. Inactive variants are removed via Ndel and Avrll digest. (B) The blue colonies contain mutants that are active on the symmetric site, therefore the white colonies are selected, containing mutants that do not recombine the symmetric site and recombine the loxF8 site. (C) A sample of the 75 white colonies where a colony PCR showed the colonies with the desired activity profile with a 1.7 KB band.

FIG. 9 shows the gating strategy to evaluate recombination efficiencies of SSRs for the HEK293T reporter cell line. (A) FACS gating strategy. Single cells were cells gated from live cells. Dead cells were gated out. From single cells, BFP+ cells, and finally mCherry+ cells were gated. (B) Schematic presentation of mRNA expression constructs and the reporter HEK293T cell line. Employed mRNA with indicated features (5′cap and polyA tail) expressing a nuclear localization signal (NLS) fused to the recombinase and the tagBFP mRNA are shown. The stable reporter cell line harbours two loxP sites (triangles) that flank a puromycin selection gene (puro). Once successfully excised by recombination, mCherry is expressed from the SFFV promoter (arrow). (C) Representative microscope images with bright field, BFP, mCherry and as overlay showing distribution of recombination. (D) FACS plots of representative mRNA transfected HEK293T reporter cell line where BFP fluorescence indicates the cells transfected and mCherry fluorescence indicates the gated BFP+ cells with a recombination event.

FIG. 10 shows an MD-based analysis of hydrogen bond and van der Waals contacts in Cre and the investigated CreK201R and CreQ311R mutants. (A) Hydrogen bond and van der Waals occupancy in the last 100 ns of MD simulations. Top panel shows loxP sequence with top (TS) (SEQ ID NO: 51) and bottom (BS) (SEQ ID NO: 139) strand labelled. (B-F) Hydrogen bond and van der Waals contacts profile from last 100 ns of MD simulations for (B) Cre, (C) CreK201R(A)-K201R(I), (D) CreQ311R(A)-Q311R(I), (E) CreK201R(A)-Q311R(I) and (F) CreQ311R(A)-K201R(I), LoxP bases in black correspond to the spacer. LoxP bases in light grey and dark grey correspond to those interacting with the protein monomer active (A) and inactive (I), respectively. Interacting protein residues from the active (A) and inactive (I) monomers are listed in light grey and dark grey, respectively. Residues involved in H-bond formation are highlighted in bold, residues involved in van der Waals contacts are highlighted in bold and underlined, and residues involved in H-bond and van der Waals contacts simultaneously are highlighted in bold with an asterisk. Phosphate groups involved in Y324 recognition are highlighted with a circle. Active versus inactive mutants are highlighted with tick and cross symbols, respectively.

FIG. 11 shows a scheme of Cre molecules binding its target loxP site. The loxP site consists of two inverted repeats (13 bp) flanking a non-palindromic spacer (8 bp). Two Cre molecules bind to the loxP site, each to one of the half-sites.

FIG. 12 represents a scheme of a typical tyrosine SSR recombination reaction. (A) Schematic representation of the stepwise recombination mechanism. Four recombinase molecules bind two DNA substrates, forming a tetrameric complex (1). Recombinases in the “cleaving competent” conformation are shown in light grey, the once in the “noncleaving” conformation are shown in dark grey. The nucleophilic tyrosine is indicated as a Yin a light grey circle and shown only for the active monomers. The activated nucleophilic tyrosine attacks the scissile phosphate (indicated as a P), forms a 3′-phosphotyrosine linkage and leads to release of free 5′OH (2). The released 5′OH attacks the neighboring phosphotyrosine, forming a HolidayJunction Intermediate and leading to strand exchange (3). The complex is isomerized and the active monomers change the conformation to inactive and vice versa (4). The cleavage and strand exchange steps are repeated (5, 6). (B) Possible outcomes of the recombination reaction. Recombination reactions can lead to excision/integration or inversion of the DNA fragment, depending on the orientation of target sites (spacer sequence). The target sites are indicated as black triangles, and their directionality indicates the orientation of the target sites. (Meinke et al., 2016).

FIG. 13 shows the mutational combinations resulting in a D7 heterotetramer with obligate catalytic activity by a bacterial recombination assay on substrates loxF8, loxF8L and loxF8R. Recombination activity of recombinases D7L+−D7R, D7L^{K201R}+D7R^{Q311R}, and D7L^{K201R}+D7R^{Q311K} are shown.

FIG. 14 shows the obligate Dre activity in bacteria visualized with a PCR-based detection method (left plasmids). Recombination activity of recombinases Dre Dre^{K202R}+Dre^{Q312R}, Dre^{K202R} and Dre^{Q312R} are shown on the rox site.

FIG. 15 shows the evolution and selection scheme: The library is created by targeting diversification of amino add positions 25, 29, 32 and 33 in the D7L recombinase and positions 69, 72 and 76 in the D7R recombinase. The two libraries are cloned together into an expression vector carrying the target site of interest, then transformed into E. coli and the recombinase is expressed overnight. The resulting recombined and non-recombined plasmids are purified to start selection. (A.) Selection against recombination on the symmetric site is carried out by selecting for inactive variants. Primer p4 binds between the target sites, amplifying only the variants inactive on the site. (B.) Selection for variants active on the site occurs in two steps. First the purified plasmid is digested with a restriction enzyme that cuts between the two target sites. Amplification will only result in a product from the active recombinases. The isolated variants with the desired activity are then cloned back into a vector carrying the target site of interest to move on to the next cycle. The selection for each new cycle was rotated between selection for active variants on loxF8 and inactive variants on loxF8I and loxF8R. (Ex: The first cycle selected for active variants on loxF8, the second cycle selected for inactive variants on loxF8L, the third cycle selected for inactive variants on loxF8R.

FIG. 16 shows an alignment of the amino add sequences of the wild-type DNA recombinases used for preparing mutated DNA recombinases according to the invention.

FIG. 17 shows the 3D structure of a wildtype Cre recombinase-loxP synapse, Pre-cleavage dimeric complex is shown in A), black highlight indicates regions of high mutational frequency in obligate Cre-like recombinases. Regions include positions 129-136, 163-181, 289-301, 310-316 and 321-324. B) shows exemplary positions in Cre dimeric complex where mutations yield obligate Cre-like recombinases (indicated with spheres), which include positions 129, 133, 173, 176, 201, 289, 292, 311, 315 and 324. Positions on the right monomeric subunit are italicized.

FIG. 18 is an overview showing single point mutations and their effect on recombinase activity of a single recombinase monomer and of a recombinase obligate dimer complex.

FIG. 19 is a sequence alignment for Cre, Vika and Dre recombinases, showing the catalytic region for each recombinase, which consists of six catalytic sites (indicated as boxes). Conserved amino acids among different recombinases are highlighted in grey,

### SEQUENCES OF EXEMPLARY RECOMBINASES

The following Table 1 provides an exemplary overview of the sequences of recombinases used in the present invention. SEQ ID NOs denoting nucleic acid sequences are cited in parentheses, e.g. SEQ ID NO: 1 denotes the amino acid sequence of wt Cre recombinase monomer, while SEQ ID NO: 26 (cited in parentheses) denotes the respective nucleic acid sequence encoding the Cre recombinase protein of SEQ ID NO: 1.

TABLE 1
Amino acid and nucleic acid sequences of recombinases

| SEQIDNO: | Name: |
|---|---|
| 1 (26) | wild type Cre recombinase monomer |
| 2 (27) | Cre recombinase monomer with K201R mutation |
| 3 (28) | Cre recombinase monomer with R282E mutation |
| 4 (29) | Cre recombinase monomer with Q311K mutation |
| 5 (30) | Cre recombinase monomer with Q311R mutation |
| 6 (31) | evolved D7L DNA recombinase monomer |
| 7 (32) | evolved D7L recombinase monomer with K201R mutation |
| 8 (33) | evolved D7L recombinase monomer with G282E mutation |
| 9 (34) | evolved D7L recombinase monomer with K25R, D29R, |
| | R32E, D33L, Q35R, E123L and R337E mutations |
| 10 (35) | evolved D7R DNA recombinase monomer |
| 11 (36) | evolved D7R recombinase monomer with Q311K mutation |
| 12 (37) | evolved D7R recombinase monomer with Q311R mutation |
| 13 (38) | evolved D7R recombinase monomer with E69D, |
| | R72K, L76E and E308R mutations |
| 14 (39) | wild type Vika DNA recombinase monomer |
| 15 (40) | Vika recombinase monomer with K219R mutation |
| 16 (41) | Vika recombinase monomer with Q330R mutation |
| 17 (42) | wild type Panto DNA recombinase monomer |
| 18 (43) | Panto recombinase monomer with K202R mutation |
| 19 (44) | Panto recombinase monomer with Q312R mutation |
| 20 (45) | wild type Dre DNA recombinase monomer |
| 21 (46) | Dre recombinase monomer with K202R mutation |
| 22 (47) | Dre recombinase monomer with Q312R mutation |
| 23 (48) | wild type Vere DNA recombinase monomer |
| 24 (49) | Vere recombinase monomer with K221R mutation |
| 25 (50) | Vere recombinase monomer with Q336R mutation |

General Definitions

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the expressions “cell”, “cell line, ” and “cell culture” are used interchangeably and all such designations include progeny. Thus, the words “transformants” and “transformed cells” include the primary subject cell and culture derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, this will be clear from the context.

The terms “polypeptide”, “peptide”, and “protein”, as used herein, are interchangeable and are defined to mean a biomolecule composed of amino acids linked by a peptide bond.

If peptide or amino acid sequences are mentioned herein, each amino acid residue is represented by a one-letter or a three-letter designation, corresponding to the trivial name of the amino acid, in accordance with the following conventional list:

| Amino Acid | One-Letter Symbol | Three-Letter Symbol |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

The terms “a”, “an” and “the” as used herein are defined to mean “one or more” and include the plural unless the context is inappropriate.

The term “about” when used in connection with a numerical value is meant to encompass numerical values within a range having a lower limit that is 5% smaller than the indicated numerical value and having an upper limit that is 5% larger than the indicated numerical value.

As used herein, the term “and/or” means that it refers to either one or both/all of the options cited in the context of this term.

The term “at least one” as used herein refers to one or more of the respective item, such as two, three, four, five, six, seven, eight, nine, ten or more than ten. According to a preferred embodiment, the term “at least one” refers to just one. In a particularly preferred embodiment relating to the number of mutations in the catalytic region of the recombinases, the term “at least one mutation” preferably means a single mutation (particularly preferably a single amino acid substitution) and no further mutations in the catalytic region.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human being sought by a researcher, veterinarian, medical doctor or other clinician, which includes alleviation of the symptoms of the disease or disorder being treated.

The term “pharmaceutical composition” as used herein refers to a substance and/or a combination of substances being used for the identification, prevention or treatment of a disease or tissue status. The pharmaceutical composition is formulated to be suitable for administration to a patient in order to prevent and/or treat a disease. Further a pharmaceutical composition refers to the combination of an active agent with a carrier, inert or active, making the composition suitable for therapeutic use. Such a carrier is also referred to as being pharmaceutically acceptable. Pharmaceutical compositions can be formulated for oral, parenteral, topical, inhalative, rectal, sublingual, transdermal, subcutaneous or vaginal application routes according to their chemical and physical properties. Pharmaceutical compositions comprise solid, semisolid, liquid, transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the carrier systems of the present invention. Semisolid compositions that can be used in the context of the invention comprise emulsion, suspension, creams, lotions, gels, globules, buccal tablets and suppositories.

As used herein, the term “pharmaceutically acceptable” embraces both human and veterinary use: For example, the term “pharmaceutically acceptable” embraces a veterinarily acceptable compound or a compound acceptable in human medicine and health care.

The “percentage of sequences identity” is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” is used herein in the context of two or more nucleic acids or polypeptide sequences, to refer to two or more sequences or subsequences that are the same, i.e. comprise the same sequence of nucleotides or amino acids. Sequences are “identical” to each other if they have a specified percentage of nucleotides or amino acid residues that are the same. According to the present invention, at least 70% identical includes at least 75%, at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity over the specified sequence, when compared and aligned for maximum correspondence over a comparison window, or designated region as measured using one of the following sequence comparison algorithms or by manual alignment and visual inspection. These definitions also refer to the complement of a test sequence. Accordingly, the term “at least 70% sequence identity” is used throughout the specification with regard to polypeptide and polynucleotide sequence comparisons. This expression preferably refers to a sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

The sequence identities disclosed herein preferably refer to the amino acid sequence or the amino acids or their positions outside the catalytic region of the recombinase and do not encompass the amino acids of or their positions in the catalytic region as identified herein.

The term “sequence comparison” is used herein to refer to the process wherein one sequence acts as a reference sequence, to which test sequences are compared. When using a sequence comparison algorithm, test and reference sequences are entered into a computer, if necessary subsequence coordinates are designated, and sequence algorithm program parameters are designated. Default program parameters are commonly used, or alternative parameters can be designated. The sequence comparison algorithm then calculates the percent sequence identities for the test sequences relative to the reference sequence, based on the program parameters. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise.

In a sequence alignment, the term “comparison window” refers to those stretches of contiguous positions of a sequence which are compared to a reference stretch of contiguous positions of a sequence having the same number of positions. Typically, the number of contiguous positions ranges from about 20 to 100 contiguous positions, from about 25 to 90 contiguous positions, from about 30 to 80 contiguous positions, from about 40 to about 70 contiguous positions, from about 50 to about 60 contiguous positions. According to the present invention, when comparing a sequence with a sequence of the present invention, such as SEQ ID NO:1, for percentage identity, preferably the whole length of the SEQ ID NO, such as the 106 amino acids of SEQ ID NO: 1, is to be compared with a reference sequence, if the reference sequence has the same length or is longer than the SEQ ID NO of the present invention. If the reference sequence is shorter than the SEQ ID NO of the present invention, the entire length of the reference sequence must be compared with the whole length of the SEQ ID NO of the present invention.

Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

A nucleic acid in the context of the present invention may be comprised in one nucleic acid molecule or may be separated into two or more nucleic acid molecules, wherein each nucleic acid molecule comprises at least one of the one or more sequences encoding the polypeptide or protein of the invention. In some embodiments, one nucleic acid molecule encodes one part or monomer of an DNA-recombining enzyme of the invention, and another nucleic acid molecule encodes another part or monomer of the DNA-recombining enzyme of the invention. In some embodiments, the nucleic acid encodes two or more DNA recombinase polypeptides. Nucleic acids encoding multiple DNA recombinase of the invention can include a nucleic acid cleavage site between two sequences encoding a DNA recombinase polypeptide, can include a transcription start site or a translation start site, such as an internal ribosomal entry site (IRES) between two sequences encoding a DNA recombinase polypeptide, and/or can encode a proteolytic target site between two or more DNA recombinase polypeptides. If two or more DNA recombinase polypeptides are encoded on one nucleic acid molecule, the two or more DNA recombinase polypeptides can be under the control of the same promoter or under the control of separate promoters.

The term “nucleic acid” refers in the context of this invention to single or double-stranded oligo- or polymers of deoxyribonucleotide or ribonucleotide bases or both. Nucleotide monomers are composed of a nucleobase, a five-carbon sugar (such as but not limited to ribose or 2′-deoxyribose), and one to three phosphate groups. Typically, a nucleic acid is formed through phosphodiester bonds between the individual nucleotide monomers, In the context of the present invention, the term nucleic acid includes but is not limited to ribonucleic acid (RNA) and deoxyribonucleic acid (DNA) molecules but also includes synthetic forms of nucleic acids comprising other linkages (e.g., peptide nucleic acids as described in Nielsen et al. (Science 254:1497-1500, 1991). Typically, nucleic acids are single- or double-stranded molecules and are composed of naturally occurring nucleotides. The depiction of a single strand of a nucleic acid also defines (at least partially) the sequence of the complementary strand. The nucleic acid may be single or double stranded or may contain portions of both double and single stranded sequences. Exemplified, double-stranded nucleic acid molecules can have 3′ or 5′ overhangs and as such are not required or assumed to be completely double-stranded over their entire length. The term nucleic acid comprises chromosomes or chromosomal segments, vectors (e.g., expression vectors), expression cassettes, naked DNA or RNA polymer, primers, probes, cDNA, genomic DNA, recombinant DNA, cRNA, mRNA, tRNA, microRNA (miRNA) or small interfering RNA (siRNA). A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated. A nucleic acid can be an isolated nucleic acid or a recombinant nucleic acid.

A nucleic acid may be present in whole cells, in a cell lysate, or may be nucleic acids in a partially purified or substantially pure form. A nucleic acid is “isolated” or “rendered substantially pure” when purified away from other cellular components or other contaminants, such as other cellular nucleic acids or proteins, by standard techniques.

The terms “vector”, “cloning vector” and “expression vector” refer to a vehicle by which a DNA or RNA sequence (e.g. a foreign gene) can be introduced into a host cell, so as to transform the host and promote expression (e.g. transcription and translation) of the introduced sequence. Various expression vectors can be employed to express the polynucleotides encoding the DNA recombinase and the DNA recombining enzyme of the present invention. Both viral-based and non-viral expression vectors can be used to produce DNA recombinase and the DNA recombining enzyme described herein e.g. in a mammalian host cell. Non-viral vectors and systems include plasmids, plasmid, cosmid, episome, artificial chromosome, phage or a viral vector. Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of antibody heavy chain and the like. For example, non-viral vectors useful for expression of polynucleotides and polypeptides described herein in mammalian (e.g. human or non-human) cells include all suitable vectors known in the art for expressing proteins. Other examples of plasmids and include replicating plasmids comprising an origin of replication, or integrative plasmids, such as for instance pUC, pcDNA, pBR, and the like.

The term “viral vector” refers to a nucleic acid vector construct that includes at least one element of viral origin and has the capacity to be packaged into a viral vector particle and encodes at least an exogenous nucleic acid. The vector and/or particle can be utilized for the purpose of transferring a nucleic acid of interest into cells either in vitro or in vivo. Numerous forms of viral vectors are known in the art. Useful viral vectors include vectors based on retroviruses, lentiviruses, adenoviruses, adeno-associated viruses, herpes viruses, vectors based on SV40, papilloma virus, Epstein Barr virus, vaccinia virus vectors, and Semliki Forest virus (SFV). Recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5, 882, 877, 6, 013, 516, 4, 861, 719, 5, 278, 056 and WO 94/19478.

The terms “recombinase”, “DNA recombinase”, “DNA recombinase enzyme” and “recombinase enzyme” are used interchangeably herein and each refers to what is understood in the field as a monomer of a protein complex allowing genetic recombination. The term includes monomeric subunits of site-specific recombinases (SSRs), in particular those derived from the tyrosine recombinase family. The functional complex comprising at least two DNA recombinases or monomers is also referred to as a “DNA recombining enzyme”. Naturally occurring DNA recombining enzymes, in particular site-specific recombinase (SSR) systems (such as tyrosine-type SSRs), usually consist of four identical monomeric subunits or monomers. Such a complex is referred to as a homotetramer.

The term “complex” of DNA recombinases or a “recombinase complex” as used herein refers to a combination of at least two monomeric recombinase subunits, also termed as recombinase enzymes. A complex of two subunits is referred to as a dimer and a complex of four subunits is referred to as a tetramer. Naturally occurring recombinase complexes consist of identical recombinase monomers or subunits, in cases of two identical subunits the complex is referred to as a homodimer, in cases of four identical subunits the complex is referred to as a homotetramer). According to a preferred embodiment of the invention, a recombinase complex comprises at least two different recombinase subunits and is therefore present as a heterodimer or as a heterotetramer.

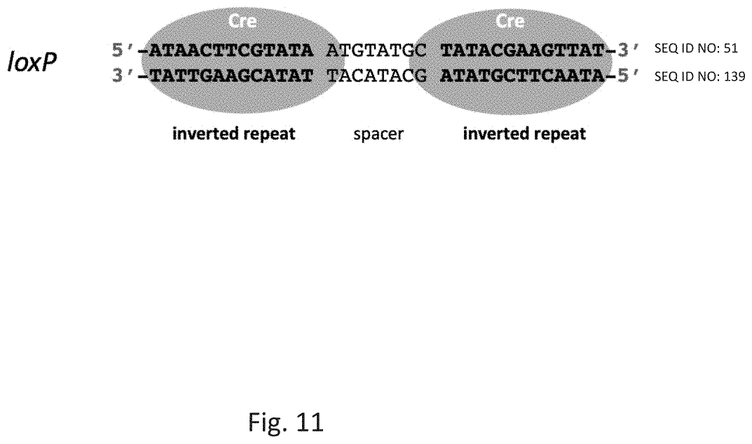

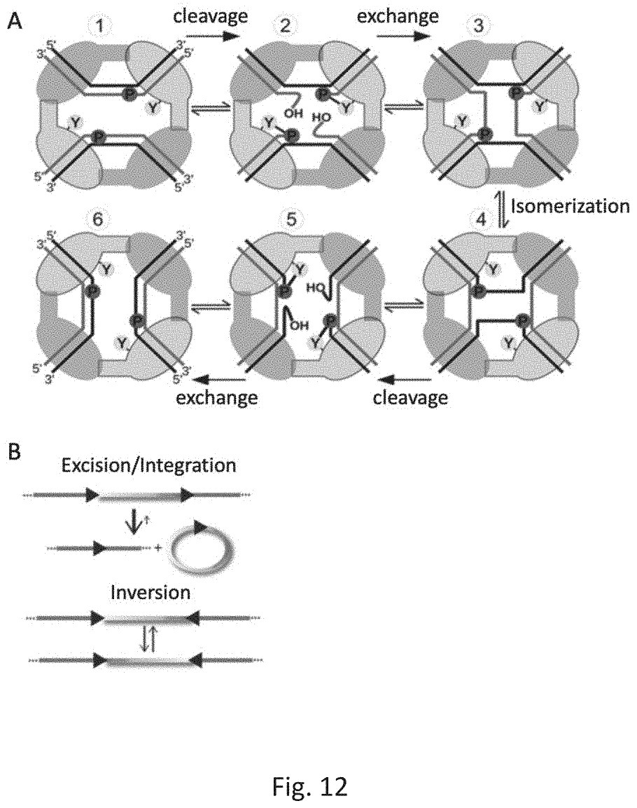

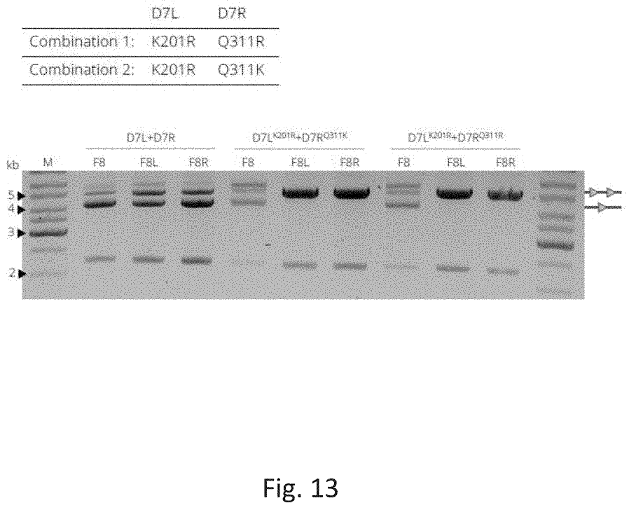

In general, such a recombinase complex modifies DNA between two specific target sequences. These sequences typically range between 30 and 200 base pairs in length and are comprised of two inversely repeated recombinase binding regions flanking a central spacer sequence where DNA breakage and replication occur (Meinke et al., 2016). An example of such a target sequence—also referred to as a target site herein—is shown in FIG. 11, which depicts the SSR Cre/loxP binding complex, where the Cre recombinase is bound to the the 34 base pair loxP target sequence. The loxP site is composed of two 13 base pair inverted repeat Cre binding elements flanking an 8 base pair spacer region. Binding elements are differentiated by their position in reference to the spacer sequence. The left half-site is the 13 base pair binding element to the left of the spacer and the right half-site is the 13 base pair binding element to the right of the spacer. Depending on the number and relative orientation of the target sites and their spacers, the DNA recombining enzyme either performs an excision, an integration, an inversion or a replacement of genetic content (FIG. 12; reviewed in Meinke et al., 2016). For a recombination event to occur, the recombinase complex recognizes a first target site and a second target site on a DNA double strand. The target sites are also referred to as upstream and downstream recognition sites, depending on their location on the DNA double strand. Accordingly, the term “half-site” as referred to herein denotes the left and right section of a DNA sequence, respectively, which are separated by a spacer sequence, and to which a recombinase enzyme binds. The half-sites are also referred to as binding regions or binding elements. The term “target site” or “target sequence” as used herein refers to a sequence element comprising a left half-site, a spacer sequence, and a right half-site.

The term “type” as used in the context of recombinases denotes a specific recombinase subunit. Preferably, said specific recombinase subunit is selected from the group consisting of Cre-, Dre-, VCre-, SCre-, Vika-, lambda-Int-, Flp-, R-, Kw-, Kd-, B2-, B3-, Nigri- and Panto-recombinases.

As used herein, the term “single mutation in the catalytic region” excludes any additional mutations in the catalytic region but does not exclude any mutations (substitutions, deletions, insertions) in the remainder of the amino acid sequence of the respective recombinase enzyme. Thus, for a recombinase having a certain percentage of identity with a given SEQ ID NO and at the same time a single amino acid substitution in its catalytic region, the percentage identity refers to the regions outside the catalytic region and does not allow any addititional mutations in the catalytic region.

As used herein, “upstream” means the 5′ target site of a recombinase in a DNA, comprising a first half-site, such as a left half-site, and a second half-site, such as a right half-site, wherein said first half-site and said second half-site are separated by a spacer sequence.

As used herein, “downstream” means the 3′ target site of a recombinase in DNA, a first half-site, such as a left half-site, and a second half-site, such as a right half-site, wherein said first half-site and said second half-site are separated by a spacer sequence.

In symmetric target sites, a first half-site, such as a left half-site, and a second half-site, such as a right half-site, are either identical or palindromic (reverse complement). In asymmetric target sites, a first half-site, such as a left half-site, and a second half-site, such as a right half-site, are not identical and not palindromic, i.e. they differ from each other in at least one nucleotide or nucleic acid.

An “obligate” protein is a complex that is composed of multiple subunits. These subunits cannot function alone and are catalytically inactive in their isolated form. When the subunits come together, they form a functional complex. Therefore, the presence of all obligate protein subunits is obligatory to the protein's functionality, i.e. the recombinase activity of the recombinase complex of the present invention.

As used herein, the term “do(es) not show the catalytic activity of a DNA recombinase” or similar terms such as “do(es) not have catalytic activity” as used in the context of a DNA recombinase enzyme having a single point mutation in its catalytic region means that said mutated DNA recombinase enzyme shows a catalytic activity of less than about 90% of the activity of the same DNA recombinase enzyme that does not have the mutation. Activity of a recombinase enzyme is preferably determined using a plasmid-based assay in E. coli. The plasmid DNA used in this assay contains two target sites of the given DNA recombinase enzyme (e.g. loxP target sites for Cre recombinase). If the DNA recombinase enzyme is active upon expression, the DNA substrate (plasmid DNA) will be recombined by the DNA recombinase enzyme. The recombination activity is calculated based on the ratio of recombined and non-recombined substrate using the following formula: Recombination activity (%)=100×(recombined substrate/(recombined+non-recombined substrates)) as the recombined and non-recombined substrate differ in size and sequence. These non-recombined and recombined DNA fragments can either be distinguished by gel electrophoresis or by sequencing.

The practice of the present invention will employ, unless otherwise indicated, conventional methods of biochemistry, cell biology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following passages, different aspects of the invention are defined in more detail. Each aspect so defined may be combined with any other aspect or aspects unless clearly indicated to the contrary. In particular, any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The invention provides a genetically engineered DNA recombining enzyme for efficient and specific genome editing, comprising an obligate complex of recombinases, wherein said complex comprises at least a first recombinase enzyme and at least a second recombinase enzyme, wherein said first recombinase enzyme and said second recombinase enzyme specifically recognize a first half-site and a second half-site of an upstream target site and/or a downstream target site of a DNA recombinase; wherein said first recombinase enzyme and said second recombinase enzyme each comprises at least one mutation in a catalytic site; wherein said first recombinase enzyme and said second recombinase enzyme carrying said at last one mutation in a catalytic site, when expressed in isolation, do not show the catalytic activity of a DNA recombinase; and wherein the catalytic activity as a DNA recombinase in said obligate complex of recombinases is complemented.

Preferably, said first half site and said second half site of the upstream target site and/or downstream target site of a DNA recombinase are not identical and are not palindromic.

The present invention further provides a genetically engineered DNA recombining enzyme comprising a complex of at least a first DNA recombinase enzyme and at least a second DNA recombinase enzyme. Said first DNA recombinase enzyme and said second DNA recombinase enzyme specifically recognize a first half-site and a second half-site of an upstream target site and/or a downstream target site of a DNA recombinase. Further, said first DNA recombinase enzyme and said second DNA recombinase enzyme each comprises a single amino acid substitution in their catalytic region, wherein said first DNA recombinase enzyme and said second DNA recombinase enzyme carrying said single amino acid substitution when expressed in isolation do not show the catalytic activity of a DNA recombinase, and wherein said first DNA recombinase enzyme and said second DNA recombinase enzyme carrying said single amino acid substitution when co-expressed and forming a complex show the catalytic activity of a DNA recombinase.

According to one embodiment, the at least one first recombinase and the at least one second recombinase are of the same type. Of the same type in this context means that the first and the second recombinase are derived from the same recombinase, which is preferably selected from the group consisting of Cre-, Dre-, VCre-, SCre-, Vika-, lambda-Int-, Flp-, R-, Kw-, Kd-, B2-, B3-, Nigri- and Panto-recombinase.

According to a particularly preferred embodiment, the DNA recombining enzyme is a complex of recombinase subunits. Such a complex may have the conformation as described herein. A preferred configuration of such a complex is a heterotetramer.

It has surprisingly been found that it is sufficient to introduce exactly one mutation in a catalytic site of each monomer of the DNA recombinase in order to inactivate the catalytic activity of said monomers, wherein, however, the catalytic activity as a DNA recombinase in the obligate complex of recombinases is complemented (restored), when two mutated monomers pair with each other. In other words, when the first recombinase with the at least one mutation in a catalytic site is co-expressed with the second recombinase with the at least one mutation in a catalytic site, and both form a heterocomplex, this heterocomplex is catalytically active and shows the activity of a DNA recombinase having no mutations in the catalytic region of its recombinase monomer subunits. Therefore, in a more preferred embodiment, the invention provides a genetically engineered DNA recombining enzyme for efficient and specific genome editing, comprising an obligate complex of recombinases, wherein said complex comprises at least a first recombinase enzyme and at least a second recombinase enzyme, wherein said first recombinase enzyme and said second recombinase enzyme specifically recognize a first half-site and a second half-site of an upstream target site and/or a downstream target site of a DNA recombinase; wherein said first recombinase enzyme and said second recombinase enzyme each comprises exactly one mutation in a catalytic site; wherein said first recombinase enzyme and said second recombinase enzyme carrying said exactly one mutation in a catalytic site, when expressed in isolation, do not show the catalytic activity of a DNA recombinase; and wherein the catalytic activity as a DNA recombinase in said obligate complex of recombinases is complemented. The first recombinase enzyme and the second recombinase enzyme may, of course, comprise further mutations which are outside the catalytic sites and/or which do not impair their catalytic activity. Preferably, said first half site and said second half site of the upstream target site and/or downstream target site of a DNA recombinase are not identical and are not palindromic.

The finding of the mutated position 201 in the first recombinase enzyme, harboring an arginine at this position rather than a lysine was most surprising. What makes this alteration so interesting is that lysine 201 is highly conserved throughout the tyrosine SSR family and it has been previously described as essential for the catalytic activity of Cre by facilitating DNA cleavage during recombination. Hence, recombinases with alterations at position 201 would not be expected to function. The mutation of the catalytic K201 residue inactivates the SSR when expressed as a monomer. On the basis of these observations, it was further surprising that the recombinase activity could be rescued by the presence of the paired Q311R mutation on the second recombinase enzyme. Only when the paired mutations are applied as single mutation in the catalytic region of each recombinase enzyme (subunit), the engineered SSRs can efficiently recombine the intended target sequence, while the recombinase enzymes (subunits) carrying the point mutations expressed in isolation are inactive.

As mentioned above, by altering the DNA-specificity of Cre through engineering and directed evolution, distinct SSR variants can be generated that together recombine asymmetric target sequences as heterotetramers (6, 8-10). The generation of such heterotetrameric SSR systems substantially broadens the potential sequences that can be targeted within genomes. However, possible combinations of different subunits could lead to active SSR byproducts capable of catalyzing off-target recombination. Previously, prevention of homotetramer formation was achieved through structure-guided redesign of several residues implicated in the protein-protein interaction interface between the different recombinase monomers (16). Hence, this approach to generate obligate SSR systems is limited to enzymes with available crystal structures and is therefore not easily adaptable to engineered or distantly related recombinases.

The invention further relates to genetically engineered DNA recombinases, which specifically recognize upstream and downstream target sequences of the loxF8 recombinase target sites, and which catalyze the inversion of a gene sequence between these upstream and downstream target sequences of the loxF8 recombinase target sites.

The invention further relates to nucleic acid molecules encoding a genetically engineered DNA recombinase according to the invention.

In a further embodiment, the invention provides a mammalian, insect, plant or bacterial host cell comprising said nucleic acid molecule or molecules encoding a genetically engineered DNA recombinase according to the invention.

A genetically engineered DNA recombinase or a nucleic acid molecule according to the invention can be used as a medicament and can therefore be comprised in a pharmaceutical composition, optionally in combination with one or more therapeutically acceptable diluents or carriers.

The genetically engineered DNA recombinase or the pharmaceutical composition according to the invention are suitable for the treatment of a disease that can be cured by genomic editing, in particular the treatment of hemophilia A.

In a further embodiment, a method for determining recombination on genomic level in a host cell culture or patient, comprising a genetically engineered DNA recombinase according to the invention, is provided.

Employing directed molecular evolution, it was surprisingly discovered by the inventors that obligate SSR systems can also be generated by mutating residues implicated in recombination catalysis. Importantly, this novel way of generating obligate SSRs only required the alteration of one conserved residue within each distinct SSR monomer. This simplified approach could potentially be applied to many engineered or natural SSRs, without prior structural knowledge of the enzymes.

The finding that the identified mutations can transform naturally occurring SSRs into obligate enzymes, including Cre recombinase, could be usefully explored for sophisticated genetics and synthetic biology studies. Numerous conditional knockout mouse models that have been generated are based on the Cre/loxP system (28, 29). Typically, animals carrying the floxed allele are crossed with mice expressing Cre from a tissue specific promoter to achieve inactivation of the gene in a particular organ or tissue. This approach could be further refined by expressing CreK201R, CreQ311K and CreQ311R from two different promoters. Here, deletion of the gene would only happen in cells where both promoters are active. In a similar fashion, further enhancement of precision for genetic lineage tracing studies (30) were achieved by employing the obligate CreK201R/CreR282E and CreQ311K/CreQ311R system. Likewise, obligate SSRs might allow for the generation of more sophisticated circuits for synthetic biology, where SSRs are frequently used to build biosensors and biological machines (31, 32).

For example, the mutations K201R, Q311K and Q311R that were identified in the Cre system to provide obligate recombinase complexes, have been shown herein to lead also to obligate complexes of other recombinases, in particular obligate complexes comprising at least two recombinase enzymes, wherein the first recombinase enzyme comprises said at least one mutation K201R, wherein the numbering of the amino acid positions refers to the amino acid sequence of the wild type Cre recombinase monomer of SEQ ID NO: 1 and wherein the second recombinase enzyme comprises said at least one mutation selected from the group constsiting of Q311K and Q311R, wherein the numbering of the amino acid positions refers to the amino acid sequence of the wild type Cre recombinase monomer of SEQ ID NO: 1.

Preferred recombinase sequences with mutations are listed in the following:

The mutated first recombinase enzyme comprising the K201R mutation has the SEQ ID NO: 2.

The mutated second recombinase enzyme comprising the Q311K mutation has the SEQ ID NO: 4.

The mutated second recombinase enzyme comprising the Q311R mutation has the SEQ ID NO: 5.

The recombinase enzymes comprised in the obligate complex are preferably DNA recombinases and may be naturally occurring recombinases (i.e. recombinases isolated from any type of biologicals samples) or designer recombinases, such as recombinases evolved by directed molecular evolution or rational design, or any combinations thereof. Methods to create designer recombinases are known in the art. WO 2018/229226 A1 for example teaches vectors and methods to generate designer DNA recombining enzymes by directed molecular evolution. WO 2008/083931 A1 discloses the directed molecular evolution of tailored recombinases (Tre 1.0) that uses sequences in the long terminal repeat (LTR) of HIV as recognition sites (loxLTR Tre 1.0). Further developments of this approach using asymmetric target sites were described in WO 2011/147590 A2 (Tre 3.0) and WO 2016/034553 A1 (Tre 3.1 and Tre/Brec1) as well as the publication Karpinski J et al 2016 (Brec1). Methods for engineering naturally occurring or designer-recombinases by rational design are also known to the art (e.g. Abi-Ghanem et al., 2013; Karimova et al., 2016).

According to a preferred embodiment of the invention, the genetically engineered DNA recombining enzyme is a mutant of a naturally occurring site-specific recombinase or a mutant of designer DNA recombinase. Naturally occurring site-specific recombinases include but are not limited to Cre-, Dre-, VCre-, SCre-, Vika-, lambda-Int-, Flp-, R-, Kw-, Kd-, B2-, B3-, Nigri- or Panto-recombinases. Designer DNA recombinase are disclosed e.g. in WO 2014/016248 and WO 2018/229226.

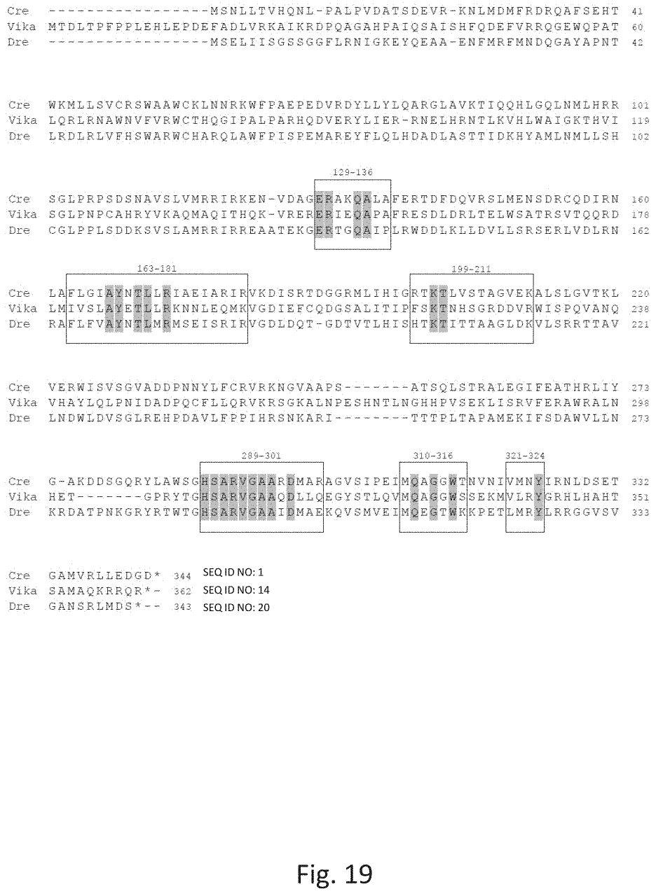

According to the present invention, the at least one and preferably the single amino acid substitution in the DNA recombinase is at a position in the catalytic region of said DNA recombinase. The catalytic region of a recombinase and specifically of a tyrosine-type SSR recombinase consists of six catalytic sites. These catalytic sites are shown in the alignment of FIG. 19 as boxes with the position numbering referring to Cre recombinase. The following Table 2 identifies those amino acids, their position in the full sequence of each DNA recombinase indicated, and the corresponding SEQ ID NO, which belong to the catalytic region.

TABLE 2
Catalytic region in exemplary DNA recombinases

| Catalytic | Cre | Vika | Dre | Panto |
|---|---|---|---|---|
| site no. | (SEQ ID NO: 1) | (SEQ ID NO: 14) | (SEQ ID NO: 20) | (SEQ ID NO: 17) |
| 1 | 129-136 | 147-154 | 131-138 | 130-137 |
| | ERAKQALA (SEQ ID | ERIEQAPA (SEQ ID | ERTGQAIP (SEQ ID NO: | ERTGQAVP (SEQ ID |
| | NO: 109) | NO: 115) | 121) | NO: 127) |
| 2 | 163-181 | 181-199 | 165-183 | 164-182 |
| | FLGIAYNTLLRIAEIARI | IVSLAYETLLRKNNLEQM | FLFVAYNTLMRMSEISRI | FLFVAYNTLCRMSELSRI |
| | R (SEQ ID NO: 110) | K (SEQ ID NO: 116) | R (SEQ ID NO: 122) | R (SEQ ID NO: 128) |
| 3 | 199-211 | 217-229 | 200-212 | 200-212 |
| | RTKTLVSTAGVEK | FSKTNHSGRDDVR (SEQ | HTKTITTAAGLDK (SEQ | HTKTMVTAAGVIK (SEQ |
| | (SEQ ID NO: 111) | ID NO: 117) | ID NO: 123) | ID NO: 129) |
| 4 | 289-301 | 308-320 | 290-302 | 290-302 |
| | HSARVGAARDMAR | HSARVGAAQDLLQ (SEQ | HSARVGAAIDMAE (SEQ | HSARVGAAMDMAE |
| | (SEQ ID NO: 112) | ID NO: 118) | ID NO: 124) | (SEQ ID NO: 130) |
| 5 | 310-316 | 329-335 | 311-317 | 311-317 |
| | MQAGGWT (SEQ ID | MQAGGWS (SEQ ID NO: | MQEGTWK (SEQ ID NO: | MQEGTWQ (SEQ ID |
| | NO: 113) | 119) | 125) | NO: 131) |
| 6 | 321-324 | 340-343 | 322-325 | 322-325 |
| | VMNY (SEQ ID NO: | VLRY (SEQ ID | LMRY (SEQ ID NO: 126) | VMRY (SEQ ID NO: |
| | 114) | NO: 120) | | 132) |

Based on the alignment of different recombinases (see for example FIG. 19), the catalytic regions in other recombinases can be identified. The skilled person will readily identify suitable alignment tools, such as the well-known tools Clustal Omega or EMBOSS Needle from the European Bioinformatics Institute of the European Molecular Biology Laboratory. For the sequence alignment shown in FIG. 19, the default settings of Clustal Omega v1.2.4 have been used. Based on such an alignment, a consensus sequence for the catalytic region can also be determined. Therefore, according to the present invention, the catalytic region can also be defined by the following amino acid sequences:

Catalytic site no. 1:
(SEQ ID NO: 133)
ER(A, I, T)(K, E, G)QA(L, P, I, V)(A, P);
Catalytic site no. 2:
(SEQ ID NO: 134)
(F, I)(L, V)(G, S, F)(I, L, V)AY(N, E)TL(L, M, C)R(I, K, M)(A, N, S)
(E, N)(I, L)(A, E, S)(R, Q)(I, M)(R, K);
Catalytic site no. 3:
(SEQ ID NO: 135)
(R, F, H)(T, S)KT(L, N, I, M)(V, H, T)(S, T)(T, G, A)(A, R)(G, D)
(V, D, L)(E, V, D, I)(K, R);
Catalytic site no. 4:
(SEQ ID NO: 136)
HSARVGAA(R, Q, I, M)D(M, L)(R, Q, E, A)(R, Q, E);
Catalytic site no. 5:
(SEQ ID NO: 137)
MQ(A, E)G(G, T)W(T, S, K, Q);
and
Catalytic site no. 6:
(SEQ ID NO: 138)
(V, L)(M, L)(N, R)Y,

wherein amino acids in parentheses denote alternative amino acids for the respective position.

According to a preferred embodiment, the at least one mutation and specifically the single amino acid substitution in the DNA recombinase is at a position of a conserved amino acid in the catalytic region of the DNA recombinase. Conserved amino acids in the catalytic region of recombinases are derivable from an alignment of recombinases such as the alignment shown in FIG. 19. Conserved amino acids in the catalytic region of recombinases preferably include positions E129, R130, Q133, A134, A167, Y168, T170, L171, L172, R173, E176, K201, T202, H289, S290, A291, R292, V293, G294, A295, A296, R297, D298, M310, Q311, G313, W315, V321, M322, N323 and Y324 of SEQ ID NO: 1, and corresponding positions in another recombinase, preferably in one or more of SEQ ID NO: 14, SEQ ID NO: 17 and SEQ ID NO: 20.

The term “an amino acid position corresponding to position . . . ” as used in the context of the present invention refers to the position of the amino acid that aligns in an alignment of amino acid sequences with an amino acid sequence of a recombinase described herein, preferably with the full length amino acid sequence of SEQ ID NO: 1. For example, a skilled person can easily align further recombinases to the alignment shown in FIG. 19, allowing the identification of amino acids corresponding to the amino acid positions identified for SEQ ID NO: 1 and likewise for SEQ ID NOs: 14, 17 and 20. For example and as derivable from the alignment, a position that corresponds to E129 of SEQ ID NO: 1 is E146 of SEQ ID NO: 14, E130 of SEQ ID NO: 17 and E131 of SEQ ID NO: 20. Further corresponding amino acid positions of other recombinase enzymes can thus be identified by the skilled person without undue burden.

According to a particularly preferred embodiment, the at least one mutation and specifically the single amino acid substitution in the DNA recombinase is at a position selected from the group consisting of position E129, Q133, R173, E176, K201, H289, R292, Q311, W315, and Y324 of SEQ ID NO: 1, or in a corresponding position of another recombinase, wherein said amino acid position in the other recombinase corresponding to position E129, Q133, R173, E176, K201, H289, R292, Q311, W315, or Y324 of SEQ ID NO: 1. Preferably, said other recombinase comprises the sequence as set forth in one of SEQ ID NO: 14, SEQ ID NO: 17 and SEQ ID NO: 20. Corresponding positions are indicated in the alignment in FIG. 19. Specifically, the corresponding positions in SEQ ID NO: 14 are at positions E146, Q151, R191, N194, K219, H308, R311, Q330, W334, and Y343, in SEQ ID NO: 17 at positions E130, Q134, R174, E177, K202, H290, R293, Q312, W316, and Y325, and in SEQ ID NO: 20 at positions E131, Q135, R175, E178, K202, H290, R293, Q312, W316, and Y325. The alignment shown in FIG. 19 enables the person of ordinary skill in the art to align further DNA recombinases and to identify the catalytic region thereof, as well as the specific positions indicated herein.

According to a particularly preferred embodiment, the single substitution in the DNA recombinase is selected from the group consisting of: E129R, Q133H, R173A, R173C, R173D, R173E, R173F, R173G, R173I, R173K, R173L, R173M, R173N, R173P, R173Q, R173S, R173T, R173V, R173W, R173Y, E176H, E176I, E176L, E176M, E176V, E176W, E176Y, K201A, K201C, K201C, K201D, K201F, K201G, K201H, K201I, K201L, K201M, K201N, K201P, K201Q, K201R, K201S, K201T, K201V, K201W, K201Y, H289D, H289E, H289I, H289K, H289R, H289W, R292A, R292C, R292E, R292F, R292G, R292H, R292I, R292L, R292M, R292N, R292P, R292Q, R292S, R292T, R292V, R292W, R292Y, Q311R, W315C, W315E, W315G, W315I, W315K, W315L, W315M, W315N, W315Q, W315R, W315S, W315T, W315V, Y324A, Y324C, Y324E, Y324F, Y324H, Y324I, Y324K, Y324L, Y324M, Y324N, Y324Q, Y324R, Y324S, Y324T, Y324V, and Y324W of SEQ ID NO: 1, or in a corresponding position of another recombinase, preferably in a corresponding position of SEQ ID NO: 14, SEQ ID NO: 17 or SEQ ID NO: 20.

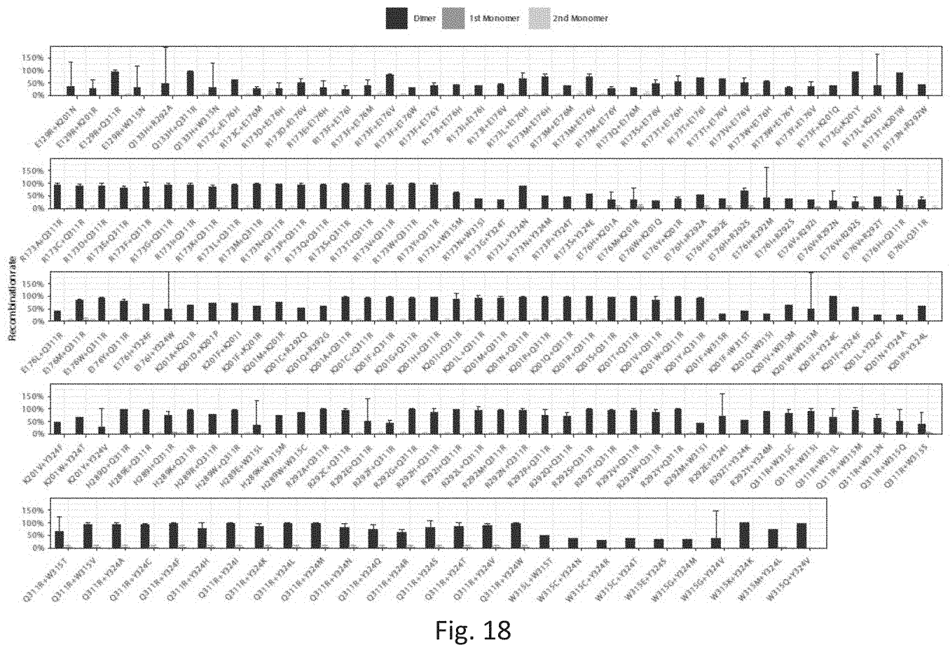

Particularly preferred combinations of single substitutions in the catalytic region of a first and second DNA recombinase are highlighted in Table 6 and in FIG. 18.

A highly preferred combination of single amino acid substitutions in a first and in a second DNA recombinase are single substitutions at positions R173 and Q311, K201 and Q311, Q311 and R292, W315 and Y324, and at positions Q311 and Y324 of SEQ ID NO: 1, respectively, or in a corresponding position of another recombinase, preferably in a corresponding position of SEQ ID NO: 14, SEQ ID NO: 17 or SEQ ID NO: 20.

In a preferred embodiment, said first recombinase enzyme comprises said at least one mutation selected from K201R, wherein the numbering of the amino acid positions refers to the amino acid sequence of the wild type Cre recombinase monomer of SEQ ID NO: 1; and said second recombinase enzyme comprises said at least one mutation at the position selected from Q311K and Q311R, wherein the numbering of the amino acid positions refers to the amino acid sequence of the wild type Cre recombinase monomer of SEQ ID NO: 1. The genetically engineered obligate DNA recombinases of the invention catalyze DNA recombination events such as excision, replacement or inversion of target sequences. The invention specifically discloses obligate complexes of recombinases, which catalyze the inversion of a DNA sequence present in the int1h regions on the human X chromosome.

In a most preferred embodiment, said first recombinase enzyme comprises said at least one or exactly one mutation at the position K201R, wherein the numbering of the amino acid position refers to the amino acid sequence of the wild type Cre recombinase monomer of SEQ ID NO: 1; and said second recombinase enzyme comprises said at least one or exactly one mutation Q311K and Q311R, wherein the numbering of the amino acid positions refers to the amino acid sequence of the wild type Cre recombinase monomer of SEQ ID NO: 1.

In a most preferred embodiment, the recombinases comprised in the complex were generated with methods described herein. Accordingly, the features and embodiments relating to the recombinase target sites and recombinases, which are subsequently described herein, also apply to described method of the invention.

A recombinase complex, e.g. also the DNA recombining enzyme of the invention, usually comprises four recombinase enzymes, i.e. four recombinase monomers. Within the scope of the invention are compositions of the recombinase complex, wherein e.g.
all four recombinase monomers are different;
three recombinase monomers are identical and one monomer is different;
two recombinase monomers are identical and the other two monomers are different;
the complex comprises two different homodimers;
the complex comprises two different heterodimers;
the complex comprises two identical heterodimers;
the complex comprises two different monomers; or
the complex comprises two identical monomers.

“Different” in this context means that the monomers are not identical in their primary structure in that they carry at least one and preferably a single amino acid substitution in their catalytic region, i.e. show differences in their amino acid sequences; and/or show a high specificity towards one of the four half-sites of an upstream target site and a downstream target site of a recombinase, which leads advantageously to a surprisingly increased specificity of the genetically engineered DNA recombining enzyme of the invention. When the DNA recombining enzyme of the invention consists of four monomers, preferably at least two monomers bear a first amino acid substitution in their catalytic region, e.g. the K201R mutation or a mutation corresponding thereto, and the other two monomers bear a second amino acid substitution in their catalytic region, e.g. the Q311K or Q311R mutation or a mutation corresponding thereto.

In a preferred embodiment, the recombinase complex is an obligate dimer, more preferably an obligate heterodimer and is preferably capable of recognizing a first target sequence and a second target sequence of an upstream or downstream recombinase target site in a DNA.

In a further preferred embodiment, the recombinase complex of the invention is a tetramer, more preferably a heterotetramer for the recognition of an upstream target site and a downstream target site of a recombinase in a DNA, wherein said tetramer consists of two obligate heterodimers as described herein, wherein the monomers of said heterodimers are preferably bound to each other, e.g. via a peptide bond or protein-protein-interaction, and wherein said obligate heterodimers show the activity of a DNA recombinase.

According to a further preferred embodiment of the present invention, the monomers of said heterodimer have been further evolved by directed evolution or rational design to specifically recognize a first half-site or second half-site of a recombinase target site. Accordingly, a first heterodimer in said complex of two obligate heterodimers specifically recognizes the first half-site and the second half-site of an upstream recombinase target site in a DNA, and a second obligate heterodimer specifically recognizes the first half-site and the second half-site of a downstream recombinase target site.

More preferably, the monomers of said heterodimer are tyrosine site-specific recombinases. Thus, a preferred embodiment of the present invention provides genetically engineered DNA recombining enzymes comprising recombinase subunits being site-specific recombinases and most preferably tyrosine site-specific recombinases.

Preferably, said tyrosine site-specific recombinases are selected from the group consisting of Cre, Dre-, VCre-, SCre-, Vika-, lambda-Int-, Flp-, R-, Kw-, Kd-, B2-, B3-, Nigri- and Panto-recombinase. The recognition target sites of these bacterial and yeast T-SSR systems have been discussed in Meinke et al., 2016 and in Karimova et al., 2016, and are shown in Table 3 below:

TABLE 3
Recognition Target Sites of Bacterial and Yeast T-SSR Systems

| T-SSR/Site | DNA target sequence (Natural host) |
|---|---|
| Cre/loxP | 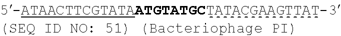 |
| Dre/rox | 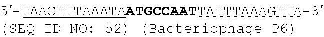 |
| MCre/Vloxp | 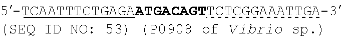 |
| SCre/SloxP | 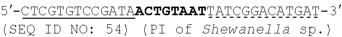 |
| Vika/vox | 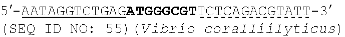 |
| Lambda-Int/ottP | 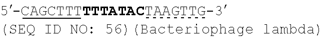 |
| FLP/FRT | 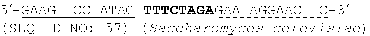 |
| R/RRT | 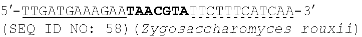 |
| Kw/KwRT | 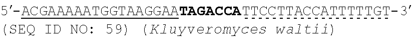 |
| Kd/KdRT | 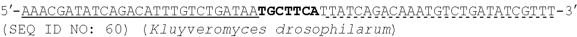 |
| B2/B2RT | 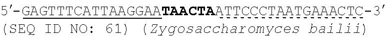 |
| B3/B3RT | 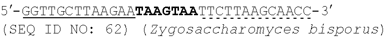 |
| Nigri/nox | 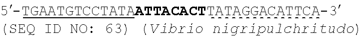 |
| Panto/pox | 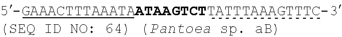 |

Solid underlined: left half-site
Dashed-underlined: right half-site
Bold: Spacer sequence

In a further preferred embodiment, a genetically engineered DNA recombining enzyme of the invention is provided, wherein said recombinase target site is a target site of a tyrosine site-specific recombinase that has been evolved by directed evolution, such as Cre-, Dre-, VCre-, SCre-, Vika-, lambda-Int-, Flp-, R-, Kw-, Kd-, B2-, B3-, Nigri- and Panto-recombinase. In a more preferred embodiment, a genetically engineered DNA recombining enzyme of the invention is provided, wherein said recombinase target site is a target site of a tyrosine site-specific recombinase that has been evolved by directed evolution, such as Cre-, Dre-, VCre-, Vika- and Panto-recombinase. Preferably, the corresponding single amino acid substitution is introduced in the catalytic region of the first recombinase enzyme and the second recombinase enzyme in the Dre-, VCre-, Vika- and Panto-recombinase the following positions:
Dre-recombinase: mutation K202R in the first recombinase enzyme and mutation Q312R in the second recombinase enzyme;
VCre-recombinase: mutation K221R in the first recombinase enzyme and mutation Q336R in the second recombinase enzyme;
Vika-recombinase: mutation K219R in the first recombinase enzyme and mutation Q330R in the second recombinase enzyme; and
Panto-recombinase: mutation K202R in the first recombinase enzyme and mutation Q312R in the second recombinase enzyme.

Most preferably, said genetically engineered DNA recombining enzyme is selected from the group comprising:
a genetically engineered DNA recombining enzyme, comprising a first recombinase enzyme and a second recombinase enzyme, wherein the first recombinase enzyme is a polypeptide which has an amino acid sequence with at least 70%, preferably 80%, more preferably 90%, sequence identity with a sequence according to SEQ ID NO: 7 and comprises the mutation corresponding to the mutation at position K201R; and wherein the second recombinase enzyme is a polypeptide which has an amino acid sequence with at least 70%, preferably 80%, more preferably 90%, sequence identity with a sequence according to SEQ ID NO: 11 and comprises the mutation corresponding to the mutation at position Q311R. In this embodiment, the mutation at position K201R was introduced into the polypeptide of SEQ ID NO: 6 and the mutation at the position Q311R was introduced into the polypeptide of SEQ ID NO: 10. The sequence identity in this context refers to the amino acid sequence outside the catalytic region of the recombinase, i.e. the catalytic region preferably only contains a single amino acid substitution and no further mutation.
a genetically engineered DNA recombining enzyme, comprising a first recombinase enzyme and a second recombinase enzyme, wherein the first recombinase enzyme is a polypeptide which has an amino acid sequence with at least 70%, preferably 80%, more preferably 90%, sequence identity with a sequence according to SEQ ID NO: 7 and comprises the mutation corresponding to the mutation at position K201R; and wherein the second recombinase enzyme is a polypeptide which has an amino acid sequence with at least 70%, preferably 80%, more preferably 90%, sequence identity with a sequence according to SEQ ID NO: 12 and comprises the mutation corresponding to the mutation at position Q311K. In this embodiment, the mutation at the position K201R was introduced into the polypeptide of SEQ ID NO: 6 and the mutation at the position Q311K was introduced into the polypeptide of SEQ ID NO: 10. The sequence identity in this context refers to the amino acid sequence outside the catalytic region of the recombinase, i.e. the catalytic region preferably only contains a single amino acid substitution and no further mutation.

According to a preferred embodiment, the mutations K201R is the only mutation in the catalytic region of the first recombinase, and the mutation Q311K or Q311R is the only mutation in the catalytic region of the second recombinase.

In a further preferred embodiment, a genetically engineered DNA recombining enzyme of the invention is provided that is a mutant of a naturally occurring site-specific recombinase selected from:
Cre recombinase, comprising the K201R mutation in the first recombinase enzyme and having an amino acid sequence which is at least 70%, preferably 80%, more preferably 90%, identical with a polypeptide having the sequence according to of SEQ ID NO: 2; and the Q311R mutation in the second recombinase enzyme having an amino acid sequence which is at least 70%, preferably 80%, more preferably 90%, identical with a polypeptide having the sequence of SEQ ID NO: 5. The wild type Cre recombinase, from which the mutated SEQ ID NOs: 2 and 5 were obtained, has the sequence of SEQ ID NO: 1, wherein the sequence identity refers to the amino acid sequence outside the catalytic region of the recombinase, i.e. the catalytic region preferably only contains a single amino acid substitution and no further mutation;
Cre recombinase, comprising the K201R mutation in the first recombinase enzyme and having an amino acid sequence which is at least 70%, preferably 80%, more preferably 90%, identical with a polypeptide having the sequence according to of SEQ ID NO: 2; and the Q311K mutation in the second recombinase enzyme having an amino acid sequence which is at least 70%, preferably 80%, more preferably 90%, identical with a polypeptide having the sequence of SEQ ID NO: 4. The wild type Cre recombinase, from which the mutated SEQ ID NOs: 2 and 4 were obtained, has the sequence of SEQ ID NO: 1, wherein the sequence identity refers to the amino acid sequence outside the catalytic region of the recombinase, i.e. the catalytic region preferably only contains a single amino acid substitution and no further mutation;
Vika recombinase, comprising the K219R mutation in the first recombinase enzyme and having an amino acid sequence which is at least 70%, preferably 80%, more preferably 90%, identical with a polypeptide having the sequence according to of SEQ ID NO: 15; and the Q330R mutation in the second recombinase enzyme having an amino acid sequence which is at least 70%, preferably 80%, more preferably 90%, identical with a polypeptide having the sequence of SEQ ID NO: 16. The wild type Vika recombinase, from which the mutated SEQ ID NOs: 15 and 16 were obtained, has the sequence of SEQ ID NO: 14, wherein the sequence identity refers to the amino acid sequence outside the catalytic region of the recombinase, i.e. the catalytic region preferably only contains a single amino acid substitution and no further mutation;
Panto recombinase, comprising the K202R mutation in the first recombinase enzyme and having an amino acid sequence which is at least 70%, preferably 80%, more preferably 90%, identical with a polypeptide having the sequence according to of SEQ ID NO: 18; and the Q312R mutation in the second recombinase enzyme having an amino acid sequence which is at least 70%, preferably 80%, more preferably 90%, identical with a polypeptide having the sequence of SEQ ID NO: 19. The wild type Panto recombinase, from which the mutated SEQ ID NOs: 18 and 19 were obtained, has the sequence of SEQ ID NO: 17, wherein the sequence identity refers to the amino acid sequence outside the catalytic region of the recombinase, i.e. the catalytic region preferably only contains a single amino acid substitution and no further mutation;
Dre recombinase, comprising the K202R mutation in the first recombinase enzyme and having an amino acid sequence which is at least 70%, preferably 80%, more preferably 90%, identical with a polypeptide having the sequence according to of SEQ ID NO: 21; and the Q312R mutation in the second recombinase enzyme having an amino acid sequence which is at least 70%, preferably 80%, more preferably 90%, identical with a polypeptide having the sequence of SEQ ID NO: 22. The wild type Dre recombinase, from which the mutated SEQ ID NOs: 21 and 22 were obtained, has the SEQ ID NO: 20, wherein the sequence identity refers to the amino acid sequence outside the catalytic region of the recombinase, i.e. the catalytic region preferably only contains a single amino acid substitution and no further mutation; and
Vcre recombinase, comprising the K221R mutation in the first recombinase enzyme and having an amino acid sequence which is at least 70%, preferably 80%, more preferably 90%, identical with a polypeptide having the sequence according to of SEQ ID NO: 24; and the Q336R mutation in the second recombinase enzyme having an amino acid sequence which is at least 70%, preferably 80%, more preferably 90%, identical with a polypeptide having the sequence of SEQ ID NO: 25. The wild type Vcre recombinase, from which the mutated SEQ ID NOs: 24 and 25 were obtained, has the SEQ ID NO: 23, wherein the sequence identity refers to the amino acid sequence outside the catalytic region of the recombinase, i.e. the catalytic region preferably only contains a single amino acid substitution and no further mutation.

According to further preferred embodiments, the first recombinase enzyme comprises the mutation selected from the group consisting of mutation K201R of SEQ ID NO: 1, 2 or 7; K202R of SEQ ID NO: 18 or 21, mutation K219R of SEQ ID NO: 15, and mutation K221R of SEQ ID NO: 24; and the second recombinase enzyme comprises the mutation selected from the group consisting of mutation Q311K of SEQ ID NO: 4 or 11, mutation Q311R of SEQ ID NO: 5 or 12, mutation Q312R of SEQ ID NO: 19 or 22, mutation Q330R of SEQ ID NO: 16, and mutation Q336R of SEQ ID NO: 25.

It is generally preferred that the genetically engineered DNA recombining enzyme according to the invention recognizes a recombinase target side, wherein said upstream recombinase target site and said downstream recombinase target site are asymmetric.

Most preferably, the genetically engineered DNA recombining enzyme according to the invention specifically recognizes the upstream recombinase target sequence of the loxF8 target site, which has the nucleic acid sequence:
ATAAATCTGTGGAAACGCTGCCACACAATCTTAG (SEQ ID NO: 65) or a reverse complement sequence thereof;

and recognizes the downstream recombinase target sequence of the loxF8 target site, which has the nucleic acid sequence
CTAAGATTGTGTGGCAGCGTTTCCACAGATTTAT (SEQ ID NO: 66) or a reverse complement sequence thereof.

Even most preferably, the genetically engineered DNA recombining enzyme according to the invention catalyzes the inversion of a gene sequence between the upstream recombinase target sequence of SEQ ID NO: 65 and the downstream recombinase target sequence of SEQ ID NO: 66 of the loxF8 recombinase target site.

The capability to catalyzes the inversion of a gene sequence between the upstream recombinase target sequence of SEQ ID NO: 65 and the downstream recombinase target sequence of SEQ ID NO: 66 of the loxF8 recombinase target site can be tested by a method comprising the steps of:
a) expressing the genetically engineered DNA recombining enzyme comprising a first recombinase enzyme and a second recombinase enzyme in a cell; and
b) analyzing, whether the genetically engineered DNA recombining enzyme expressed in step a) is capable of inverting of DNA sequence on a human chromosome in said cell.

Preferred genetically engineered DNA recombining enzymes or recombinase complexes according to the invention, that are capable of catalyzing the inversion of a gene sequence between the upstream recombinase target sequence of SEQ ID NO: 65 and the downstream recombinase target sequence of SEQ ID NO: 66 of the loxF8 recombinase target site, are DNA recombining enzymes, wherein the first recombinase enzyme has an amino acid sequence with at least 70%, preferably 80%, more preferably 90%, sequence identity with a sequence according to SEQ ID NO: 7 or SEQ ID NO: 8 and/or wherein said second recombinase enzyme has an amino acid sequence with at least 70%, preferably 80%, more preferably 90%, sequence identity with a sequence according to SEQ ID NO: 11 or SEQ ID NO: 12.

The most preferred genetically engineered DNA recombining enzyme or recombinase complex according to the invention, that is capable of catalyzing the inversion of a gene sequence between the upstream recombinase target sequence of SEQ ID NO: 65 and the downstream recombinase target sequence of SEQ ID NO: 66 of the loxF8 recombinase target site comprises a first recombinase enzyme having an amino acid sequence with at least 70%, preferably 80%, more preferably 90%, sequence identity with the sequence according to SEQ ID NO: 7 and a second recombinase enzyme having an amino acid sequence with at least 70%, preferably 80%, more preferably 90%, sequence identity with the sequence according to SEQ ID NO: 11, wherein the sequence identity preferably refers to the amino acid sequence outside the catalytic region of the recombinase.

To generate DNA recombinases that recombine the upstream and downstream target sequences of the loxF8 target site, the substrate linked directed evolution approach can be employed (Buchholz and Stewart 2001). In the so evolved DNA recombinases, the mutation at the position corresponding to the mutation K201R or G282E or R282E of a first Cre recombinase monomer of SEQ ID NO: 1, and at the position corresponding to the mutation Q311K or Q311R of a second Cre recombinase monomer of SEQ ID NO: 1 were introduced, respectively, resulting in the genetically engineered recombinase monomers of SEQ ID NOs: 2, 3, 4 and 5 of the invention.

Said genetically engineered DNA recombining enzyme according to the invention recombines a nucleic acid sequence, in particular a DNA sequence by recognizing two target sites and causing a deletion, an insertion, an inversion or a replacement of a DNA sequence. Advantageously, the genetically engineered DNA recombining enzyme according to the invention recognizes the asymmetric recognition sites of the loxF8 sequence according to SEQ ID No. 65 (upstream) and SEQ ID NO: 66 (downstream). These recognition sites do not occur anywhere else in the human genome and therefore can be used for specific DNA recombination. The genetically engineered DNA recombining enzyme according to the invention advantageously does not need to target sites that are artificially introduced in the genome. Further advantageously and most preferably, the genetically engineered DNA recombining enzyme according to the invention causes an inversion of a DNA sequence. A further advantage is that the genetically engineered DNA recombining enzyme according to the invention allows precise genome editing without triggering endogenous DNA repair pathways. The invention further relates to a nucleic acid molecule, such as a polynucleotide or nucleic acid or a plurality of nucleic acid molecules each comprising or consisting of a nucleic acid sequence encoding a DNA recombinase, such as a first and a second DNA recombinase, or a genetically modified DNA recombining enzyme or a subunit thereof according to the invention.

The coding sequence, which encodes the polypeptide may be identical to the coding sequence for the polypeptides shown in SEQ ID NOs: 27-30, 32, 33, 36, 37, 40, 41, 43, 44, 46, 47, 49 and 50, preferably of SEQ ID NOs: 27-30, 32, 33, 36 and 37, or it may be a different coding sequence encoding the same polypeptide, as a result of the redundancy or degeneracy of the genetic code or a single nucleotide polymorphism.

For example, it may also be an RNA transcript of SEQ ID Nos: 27-30, 32, 33, 36, 37, 40, 41, 43, 44, 46, 47, 49 and 50, which includes the entire length of a coding sequence for a polypeptide of the invention. In a preferred embodiment, the “polynucleotide” according to the invention is one of SEQ ID NOs: 27-30, 32, 33, 36 and 37.

The wild type or original polypeptides, which have been used for genetic engineering according to the invention, are encoded by the following polynucleotides:
the polynucleotide of SEQ ID NO: 26 encodes a wild type Cre DNA recombinase monomer of SEQ ID NO: 1;
the polynucleotide of SEQ ID NO: 31 encodes the evolved D7L DNA recombinase monomer of SEQ ID NO: 6;
the polynucleotide of SEQ ID NO: 35 encodes the evolved D7R DNA recombinase monomer of SEQ ID NO: 10;
the polynucleotide of SEQ ID NO: 39 encodes a wild type Vika DNA recombinase monomer of SEQ ID NO: 14;
the polynucleotide of SEQ ID NO: 42 encodes a wild type Panto DNA recombinase monomer of SEQ ID NO: 17;
the polynucleotide of SEQ ID NO: 45 encodes a wild type Dre DNA recombinase monomer of SEQ ID NO: 20; and
the polynucleotide of SEQ ID NO: 48 encodes a wild type Cre DNA recombinase monomer of SEQ ID NO: 23.

The nucleic acids which encode the polypeptides of SEQ ID NOs: 2-5, 7, 8, 11, 12, 15, 16, 18, 19, 21, 22, 24 or 25, preferably of SEQ ID NOs: 2-5, 7, 8, 11 or 12 may include but are not limited to the coding sequence for the polypeptide alone; the coding sequence for the polypeptide plus additional coding sequence, such as a leader or secretory sequence or a proprotein sequence; and the coding sequence for the polypeptide (and optionally additional coding sequence) plus non-coding sequence, such as introns or a non-coding sequence 5′ and/or 3′ of the coding sequence for the polypeptide. The nucleic acids which encode the polypeptides of SEQ ID NOs: 2-5, 7, 8, 11, 12, 15, 16, 18, 19, 21, 22, 24 or 25, preferably of SEQ ID NOs: 2-5, 7, 8, 11 or 12 include nucleic acids, which have been codon-optimized for expression in human cells. They may further contain a nuclear localization sequence.

Thus, the term “polynucleotide encoding a polypeptide” or the term “nucleic acid encoding a polypeptide” should be understood to encompass a polynucleotide or nucleic acid which includes only a coding sequence for a DNA recombinase enzyme of the invention, e.g. a polypeptide selected from SEQ ID NOs: 2-5, 7, 8, 11, 12, 15, 16, 18, 19, 21, 22, 24 or 25, preferably of SEQ ID NOs: 2-5, 7, 8, 11 or 12 as well as one which includes additional coding and/or non-coding sequence. The terms polynucleotides and nucleic acid are used interchangeably.

The present invention also includes polynucleotides in which the coding sequence for the polypeptide may be fused in the same reading frame to a polynucleotide sequence which aids in expression and secretion of a polypeptide from a host cell; for example, a leader sequence which functions as a secretory sequence for controlling transport of a polypeptide from the cell may be so fused. The polypeptide having such a leader sequence is termed a preprotein or a preproprotein and may have the leader sequence cleaved, by the host cell to form the mature form of the protein. These polynucleotides may have a 5′ extended region so that it encodes a proprotein, which is the mature protein plus additional amino acid residues at the N-terminus. The expression product having such a prosequence is termed a proprotein, which is an inactive form of the mature protein; however, once the prosequence is cleaved, an active mature protein remains. The additional sequence may also be attached to the protein and be part of the mature protein. Thus, for example, the polynucleotides of the present invention may encode polypeptides, or proteins having a prosequence, or proteins having both a prosequence and a presequence (such as a leader sequence).

The polynucleotides of the present invention may also have the coding sequence fused in frame to a marker sequence, which allows for purification of the polypeptides of the present invention. The marker sequence may be an affinity tag or an epitope tag such as a polyhistidine tag, a streptavidin tag, a Xpress tag, a FLAG tag, a cellulose or chitin binding tag, a glutathione-S transferase tag (GST), a hemagglutinin (HA) tag, a c-myc tag or a V5 tag.

The HA tag would correspond to an epitope obtained from the influenza hemagglutinin protein (Wilson et al., 1984), and the c-myc tag may be an epitope from human Myc protein (Evans et al., 1985).

If the nucleic acid of the invention is a mRNA, in particular for use as a medicament, the delivery of mRNA therapeutics has been facilitated by significant progress in maximizing the translation and stability of mRNA, preventing its immune-stimulatory activity and the development of in vivo delivery technologies. The 5′ cap and 3′ poly(A) tail are the main contributors to efficient translation and prolonged half-life of mature eukaryotic mRNAs. Incorporation of cap analogs such as ARCA (anti-reverse cap analogs) and poly(A) tail of 120-150 bp into in vitro transcribed (IVT) mRNAs has markedly improved expression of the encoded proteins and mRNA stability. New types of cap analogs, such as 1, 2-dithiodiphosphate-modified caps, with resistance against RNA decapping complex, can further improve the efficiency of RNA translation. Replacing rare codons within mRNA protein-coding sequences with synonymous frequently occurring codons, so-called codon optimization, also facilitates better efficacy of protein synthesis and limits mRNA destabilization by rare codons, thus preventing accelerated degradation of the transcript. Similarly, engineering 3′ and 5′ untranslated regions (UTRs), which contain sequences responsible for recruiting RNA-binding proteins (RBPs) and miRNAs, can enhance the level of protein product. Interestingly, UTRs can be deliberately modified to encode regulatory elements (e.g., K-turn motifs and miRNA binding sites), providing a means to control RNA expression in a cell-specific manner. Some RNA base modifications such as N1-methyl-pseudouridine have not only been instrumental in masking mRNA immune-stimulatory activity but have also been shown to increase mRNA translation by enhancing translation initiation. In addition to their observed effects on protein translation, base modifications and codon optimization affect the secondary structure of mRNA, which in turn influences its translation. Respective modifications of the nucleic acid molecules of the invention are also contemplated by the invention.

The RNA or plurality of RNAs preferably encode the DNA recombining enzyme or any of its subunits. Specific methods for delivering and expressing nucleic acids and specifically RNAs are disclosed e.g. in EP2590676 and EP3115064. The RNA may be present in a particle and is preferably self-replicating. After in vivo administration of the particles, RNA is released from the particles and is translated inside a cell to provide the DNA recombining enzyme or any of its monomeric subunits.

A self-replicating RNA molecule (replicon) can, when delivered to a vertebrate cell even without any proteins, lead to the production of multiple daughter RNAs by transcription from itself (via an antisense copy which it generates from itself). These daughter RNAs, as well as collinear subgenomic transcripts, may be translated by themselves to provide in situ expression of an encoded polypeptide, or may be transcribed to provide further transcripts with the same sense as the delivered RNA which are translated to provide in situ expression of the polypeptide. The overall results of this sequence of transcriptions is a huge amplification in the number of the introduced replicon RNAs and so the encoded polypeptide becomes a major polypeptide product of the cells.

A preferred self-replicating RNA molecule encodes (i) a RNA-dependent RNA polymerase which can transcribe RNA from the self-replicating RNA molecule and (ii) a polypeptide of the present invention. The polymerase can be an alphavirus replicase e.g. comprising one or more of alphavirus proteins nsP1, nsP2, nsP3 and nsP4. It is preferred that the self-replicating RNA molecules of the invention do not encode alphavirus structural proteins. Thus a preferred self-replicating RNA can lead to the production of genomic RNA copies of itself in a cell, but not to the production of RNA-containing virions. A self-replicating RNA molecule useful in the context of the present invention may have two open reading frames. The first (5′) open reading frame encodes a replicase, and the second (3′) open reading frame encodes a polypeptide of the present invention. In some embodiments the RNA may have additional (e.g. downstream) open reading frames e.g. for further encoding accessory polypeptides.

Such RNA is particularly suitable for the general use in gene therapy, and specifically for use in the treatment of genetic disorder or disease.

The present invention is considered to further provide polynucleotides which hybridize to the hereinabove-described sequences wherein there is at least 70%, preferably at least 90%, and more preferably at least 95% identity or similarity between the sequences, and thus encode proteins having similar biological activity. Moreover, as known in the art, there is “similarity” between two polypeptides when the amino acid sequences contain the same or conserved amino acid substitutes for each individual residue in the sequence. Identity and similarity may be measured using sequence analysis software (e.g., ClustalW at PBIL (Pôle Bioinformatique Lyonnais) http://npsa-pbil.ibcp.fr). The present invention particularly provides such polynucleotides, which hybridize under stringent conditions to the hereinabove-described polynucleotides.

Suitably stringent conditions can be defined by, e.g., the concentration of salt or formamide in the prehybridization and hybridization solution, or by the hybridization temperature, and are well known in the art. In particular, stringency can be increased by reducing the concentration of salt, by increasing the concentration of formamide, and/or by raising the hybridization temperature.

For example, hybridization under high stringency conditions may employ about 50% formamide at about 37° C. to 42° C., whereas hybridization under reduced stringency conditions might employ about 35% to 25% formamide at about 30° C. to 35° C. One particular set of conditions for hybridization under high stringency conditions employs 42° C., 50% formamide, 5× SSPE, 0.3% SDS, and 200 μg/ml sheared and denatured salmon sperm DNA. For hybridization under reduced stringency, similar conditions as described above may be used in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art. Preferably, hybridization should occur only if there is at least 95%, and more preferably at least 97%, identity between the sequences. The polynucleotides which hybridize to the hereinabove described polynucleotides in a preferred embodiment encode polypeptides which exhibit substantially the same biological function or activity as the mature protein of SEQ ID NOs: 2-5, 7, 8, 11, 12, 15, 16, 18, 19, 21, 22, 24 or 25, preferably of SEQ ID NOs: 2-5, 7, 8, 11 or 12.

As mentioned, a suitable polynucleotide probe may have at least 14 bases, preferably 30 bases, and more preferably at least 50 bases, and will hybridize to a polynucleotide of the present invention, which has an identity thereto, as hereinabove described. For example, such polynucleotides may be employed as a probe for hybridizing to the polynucleotides encoding the polypeptides of SEQ ID NOs: 2-5, 7, 8, 11, 12, 15, 16, 18, 19, 21, 22, 24 or 25, preferably of SEQ ID NOs: 2-5, 7, 8, 11 or 12, for example, for recovery of such a polynucleotide, or as a diagnostic probe, or as a PCR primer. Thus, the present invention includes polynucleotides having at least a 70% identity, preferably at least a 90% identity, and more preferably at least a 95% identity to a polynucleotide of SEQ ID Nos: 27-30, 32, 33, 36, 37, 40, 41, 43, 44, 46, 47, 49 and 50, which encodes a polypeptide of SEQ ID NOs: 2-5, 7, 8, 11, 12, 15, 16, 18, 19, 21, 22, 24 or 25, preferably of SEQ ID NOs: 2-5, 7, 8, 11 or 12, as well as fragments thereof, which fragments preferably have at least 30 bases and more preferably at least 50 bases.

The terms “homology” or “identity, ” as used interchangeably herein, refer to sequence similarity between two polynucleotide sequences or between two polypeptide sequences, with identity being a more strict comparison. The phrases “percent identity or homology” and “identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that can be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences.

A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantially identical, ” as used herein, refers to an identity or homology of at least 70%, 75%, at least 80%, at least 85%, at least 90%, 91%, 92%, 93%, 94%, 95%, 96%, 97%, 98%, 99%, or more.

The degree of sequence identity is determined by choosing one sequence as the query sequence and aligning it with the internet-based tool ClustalW with homologous sequences taken from GenBank using the blastp algorithm (NCBI).

As it is well known in the art, the genetic code is redundant in that certain amino acids are coded for by more than one nucleotide triplet (codon), and the invention includes those polynucleotide sequences which encode the same amino acids using a different codon from that specifically exemplified in the sequences herein. Such a polynucleotide sequence is referred to herein as an “equivalent” polynucleotide sequence. The present invention further includes variants of the hereinabove described polynucleotides which encode for fragments, such as part or all of the protein, analogs and derivatives of a polypeptide of the amino acid sequences disclosed herein, preferably of SEQ ID NOs: 2-5, 7, 8, 11 or 12. The variant forms of the polynucleotide may be a naturally occurring allelic variant of the polynucleotide or a non-naturally occurring variant of the polynucleotide. For example, the variant in the nucleic acid may simply be a difference in codon sequence for the amino acid resulting from the degeneracy of the genetic code, or there may be deletion variants, substitution variants and addition or insertion variants. As known in the art, an allelic variant is an alternative form of a polynucleotide sequence, which may have a substitution, deletion or addition of one or more nucleotides that does not substantially alter the biological function of the encoded polypeptide.

In further embodiment, the polynucleotide of the invention encodes an obligate heterodimer, wherein said heterodimer comprises a first recombinase enzyme, which has an amino acid sequence with at least 70%, preferably 80%, more preferably 90%, sequence identity with a sequence according to SEQ ID NO: 2, 3, 7, 8, 15, 18, 21 or 24 and a second recombinase enzyme, which has the amino acid sequence with at least 70%, preferably 80%, more preferably 90%, sequence identity with a sequence according to SEQ ID NO: 4, 5, 11, 12, 16, 19, 22 or 25 for the recognition of an upstream target sequence and a downstream target sequence of a recombinase target site.

In one embodiment, the polynucleotide of the invention comprises a nucleic acid of SEQ ID NO: 27 and a nucleic acid of SEQ ID NO: 29.

In a further embodiment, the polynucleotide of the invention comprises a nucleic acid of SEQ ID NO: 27 and a nucleic acid of SEQ ID NO: 30.

In a further embodiment, the polynucleotide of the invention comprises a nucleic acid of SEQ ID NO: 28 and a nucleic acid of SEQ ID NO: 30.

In a further embodiment, the polynucleotide of the invention comprises a nucleic acid of SEQ ID NO: 32 and a nucleic acid of SEQ ID NO: 36.

In a further embodiment, the polynucleotide of the invention comprises a nucleic acid of SEQ ID NO: 32 and a nucleic acid of SEQ ID NO: 37.

In a further embodiment, the polynucleotide of the invention comprises a nucleic acid of SEQ ID NO: 33 and a nucleic acid of SEQ ID NO: 36.

In a further embodiment, the polynucleotide of the invention comprises a nucleic acid of SEQ ID NO: 40 and a nucleic acid of SEQ ID NO: 41.

In a further embodiment, the polynucleotide of the invention comprises a nucleic acid of SEQ ID NO: 43 and a nucleic acid of SEQ ID NO: 44.

In a further embodiment, the polynucleotide of the invention comprises a nucleic acid of SEQ ID NO: 46 and a nucleic acid of SEQ ID NO: 47.

In a further embodiment, the polynucleotide of the invention comprises a nucleic acid of SEQ ID NO: 49 and a nucleic acid of SEQ ID NO: 50.

The present invention also provides vectors, preferably expression vectors, which include such polynucleotides, host cells which are genetically engineered with such vectors, or which comprise the nucleic acid molecule or the plurality of nucleic acid molecules according to the invention, as well as the production of the polypeptides of the invention such as SEQ ID NOs: 2-5, 7, 8, 11, 12, 15, 16, 18, 19, 21, 22, 24 or 25, preferably of SEQ ID NOs: 2-5, 7, 8, 11 or 12 by recombinant techniques. Host cells are genetically engineered (transduced or transformed or transconjugated or transfected) with such vectors, which may be, for example, a cloning vector or an expression vector. The vector may be, for example, in the form of a plasmid, a conjugative plasmid, a viral particle, a phage, etc. The vector or the gene may be integrated into the chromosome at a specific or a not specified site. Methods for genome integration of recombinant DNA, such as homologous recombination or transposase-mediated integration, are well known in the art. The engineered host cells can be cultured in conventional nutrient media modified as appropriate for activating promoters, selecting transformants or amplifying the genes of the present invention. The culture conditions, such as temperature, pH and the like, are those commonly used with the host cell selected for expression, as well known to the ordinarily skilled artisan. The host cell can be a mammalian, insect, plant or bacterial host cell, comprising a nucleic acid or a recombinant polynucleotide molecule or an expression vector described herein.

The polynucleotide sequence in the expression vector is operatively linked to an appropriate expression control sequence(s) (promoter) to direct mRNA synthesis. As non-limiting and representative examples of such promoters, there may be mentioned: LTR or SV40 promoter, the E. coli lac, ara, rha or trp, the phage lambda PL promoter and other promoters known to control expression of genes in prokaryotic or eukaryotic cells or their viruses.

One skilled in the art can select a vector based on desired properties, for example, for production of a vector in a particular cell such as a mammalian cell or a bacterial cell.

Any of a variety of inducible promoters or enhancers can be included in the vector for expression of an antibody of the invention or nucleic acid that can be regulated. Such inducible systems, include, for example, tetracycline inducible System; metallothionein promoter induced by heavy metals; insect steroid hormone responsive to ecdysone or related steroids such as muristerone; mouse mammary tumor virus (MMTV) induced by steroids such as glucocorticoid and estrogen; and heat shock promoters inducible by temperature changes; the rat neuron specific enolase gene promoter; the human β-actin gene promoter; the human platelet derived growth factor B (PDGF-B) chain gene promoter; the rat sodium channel gene promoter; the human copper-zinc superoxide dismutase gene promoter; and promoters for members of the mammalian POU-domain regulatory gene family.

Regulatory elements, including promoters or enhancers, can be constitutive or regulated, depending upon the nature of the regulation. The regulatory sequences or regulatory elements are operatively linked to one of the polynucleotide sequences of the invention such that the physical and functional relationship between the polynucleotide sequence and the regulatory sequence allows transcription of the polynucleotide sequence. Vectors useful for expression in eukaryotic cells can include, for example, regulatory elements including the CAG promoter, the SV40 early promoter, the cytomegalovirus (CMV) promoter, the mouse mammary tumor virus (MMTV) steroid-inducible promoter, Pgtf, Moloney marine leukemia virus (MMLV) promoter, thy-1 promoter and the like.

If desired, the vector can contain a selectable marker. As used herein, a “selectable marker” refers to a genetic element that provides a selectable phenotype to a cell in which the selectable marker has been introduced. A selectable marker is generally a gene whose gene product provides resistance to an agent that inhibits cell growth or kills a cell. A variety of selectable markers can be used in the DNA constructs of the invention, including, for example, Neo, Hyg, hisD, Gpt and Ble genes, as described, for example in Ausubel et al., 1999 and U.S. Pat. No. 5, 981, 830. Drugs useful for selecting for the presence of a selectable marker include, for example, G418 for Neo, hygromycin for Hyg, histidinol for hisD, xanthine for Gpt, and bleomycin for Ble. DNA constructs of the invention can incorporate a positive selectable marker, a negative selectable marker, or both.

Various mammalian cell culture systems can also be employed to express a recombinant protein. Examples of mammalian expression systems include the COS-7 lines of monkey kidney fibroblasts. Other cell lines capable of expressing a compatible vector include, for example, the C127, 3T3, CHO, HeLa and BHK cell lines. Mammalian expression vectors will generally comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide required nontranscribed genetic elements.

The polypeptides can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Recovery can be facilitated if the polypeptide is expressed at the surface of the cells, but such is not a prerequisite. Recovery may also be desirable of cleavage products that are cleaved following expression of a longer form of the polypeptide. Protein refolding steps as known in this art can be used, as necessary, to complete configuration of the mature protein. High performance liquid chromatography (HPLC) can be employed for final purification steps.

In accordance with a further embodiment of the invention, there are provided gene therapy vectors, e.g., for use in systemically or locally increasing the expression of the genetically engineered proteins of the invention in a subject. The gene therapy vectors find use in preventing, mitigating, ameliorating, reducing, inhibiting, and/or treating a disease that can be treated by genome editing, in particular of hemophilia A. The gene therapy vectors typically comprise an expression cassette comprising a polynucleotide encoding a genetically engineered DNA recombining enzyme of the invention. In one embodiment, the vector is a viral vector. In a preferred embodiment, the viral vector is from a virus selected from the group consisting of adenovirus, retrovirus, lentivirus, herpesvirus and adeno-associated virus (AAV). In a more preferred embodiment, the vector is from one or more of adeno-associated virus (AAV) serotypes 1-11, or any subgroups or any engineered forms thereof. In another embodiment, the viral vector is encapsulated in an anionic liposome.

In another embodiment, the vector is a non-viral vector. In a preferred embodiment, the non-viral vector is selected from the group consisting of naked DNA, a cationic liposome complex, a cationic polymer complex, a cationic liposome-polymer complex, and an exosome.

If the vector is a viral vector, the expression cassette suitably comprises operably linked in the 5′ to 3′ direction (from the perspective of the mRNA to be transcribed) a first inverse terminal repeat, an enhancer, a promoter, the polynucleotide encoding a genetically engineered DNA recombining enzyme of the invention, a 3′ untranslated region, polyadenylation (polyA) signal, and a second inverse terminal repeat. The promoter is e.g. selected from the group consisting of cytomegalovirus (CMV) promoter and chicken-beta actin (CAG) promoter. The polynucleotide comprises preferably DNA or cDNA or RNA or mRNA. In a preferred embodiment, the polynucleotide encoding a genetically engineered DNA recombining enzyme of the invention comprises one or more of the polynucleotides of SEQ ID NOs: 27-30, 32, 33, 36, 37, 40, 41, 43, 44, 46, 47, 49 and 50. In a most preferred embodiment, the polynucleotide encoding a genetically engineered DNA recombining enzyme of the invention has at least about 75%, 80%, 85% or 90% sequence identity, e.g. at least about 91%, 92%, 93%, 94%, 95%, 96%, 97%, 98%, 99% sequence identity, to one or more of SEQ ID NOs: 27-30, 32, 33, 36 and 37.

The invention further relates to the genetically engineered DNA recombining enzyme of the invention or the nucleic acid molecule or plurality of nucleic acid molecules, or the recombinant polynucleotide, or the expression vector of the invention for use as a medicament. In a more preferred embodiment, the invention further relates to the genetically engineered DNA recombining enzyme of the invention, or the nucleic acid molecule or plurality of nucleic acid molecules, or the recombinant polynucleotide, or the expression vector of the invention for use in the prevention or treatment of a disease that can be treated by genome editing, in particular hemophilia A.

In a further embodiment, the invention relates to the use of a genetically engineered DNA recombining enzyme of the invention or the nucleic acid molecule or plurality of nucleic acid molecules, or the recombinant polynucleotide, or the expression vector of the invention for the preparation of a medicament for the prevention or treatment of a disease that can be treated by genome editing. According to a preferred embodiment, said disease is a genetic disease or disorder. According to a particularly preferred embodiment, said disease or disorder is hemophilia A.

In a further embodiment, the invention relates to a method of prevention or treatment of a disease that can be treated by genome editing, in particular hemophilia A, comprising administering a therapeutically effective amount of a genetically engineered DNA recombining enzyme of the invention or of the nucleic acid molecule or plurality of nucleic acid molecules, or the recombinant polynucleotide, or the expression vector of the invention to a patient in need thereof.

The genetically engineered DNA recombining enzyme of the invention, or the nucleic acid molecule or plurality of nucleic acid molecules, or the recombinant polynucleotide, or the expression vector of the invention can be used for treating in particular genetic disorders or diseases resulting from genetic disorders. A particularly preferred embodiment relates to the treatment of severe forms of hemophilia A.

The present invention also provides methods for generating a complex of obligate DNA recombinase enzymes. According to a preferred embodiment, such a method comprises the steps of:

(i) introducing a single amino acid substitution into the catalytic region of a first DNA recombinase enzyme, wherein said single amino acid substitution renders the first DNA recombinase enzyme catalytically inactive;

(ii) introducing a single amino acid substitution into the catalytic region of a second DNA recombinase enzyme, wherein said single amino acid substitution renders the second DNA recombinase enzyme catalytically inactive;

(iii) co-expressing both the mutated first and the mutated second DNA recombinase enzymes in a host cell;

(iv) isolating the mutated first and the mutated second DNA recombinase enzymes from the host cell.

The present invention further provides a method for generating obligate DNA recombinases for genome editing, preferably for recombination of DNA sequences, comprises the steps of:
i. providing a nucleic acid molecule encoding a first recombinase enzyme and a nucleic acid molecule encoding a second recombinase enzyme, wherein said first recombinase enzyme binds to a first half site of an asymmetric recombinase target site and said second recombinase enzyme binds to a second half site of an asymmetric recombinase target site, wherein said first recombinase enzyme and said second recombinase enzyme form a heterodimer, which is capable to induce a site-specific DNA recombination of a sequence of interest at said asymmetric recombinase target site in a DNA sequence, wherein said asymmetric recombinase target site comprises a first and a second half site of a recombinase target site, which are not identical and not palindromic;
ii. mutagenesis to create libraries of nucleic acid molecules encoding mutant first recombinase enzymes and of nucleic acid molecules encoding mutant second recombinase enzymes, wherein mutations are introduced in said first recombinase enzyme and said second recombinase enzyme;
iii. creating expression vectors, by cloning the library of the nucleic acid molecules encoding a first mutant recombinase enzyme and the library of nucleic acid molecules encoding a second mutant recombinase enzyme into expression vectors, wherein said expression vectors carry a DNA sequence of interest, which is to be recombined;
iv. transfecting a cell with the expression vectors of step iii) and expressing the libraries of said mutant first recombinase enzyme and said mutant second recombinase enzyme in the same cell resulting in the formation of recombinase heterodimers comprising a mutant first recombinase enzyme and a mutant second recombinase enzyme;
v. positive selection screen for heterodimers obtained in step iv. that are capable to induce a site-specific DNA recombination of a sequence of interest at an asymmetric recombinase target site in a DNA;
vi. negative selection screen for heterodimers obtained in step iv. or v. that are not capable to induce a site-specific DNA recombination of a sequence of interest at an off-target, preferably symmetric, recombinase target site in a DNA;
vii. selecting an obligate DNA recombinase which is capable of recombining a DNA sequence of interest at a recombinase target site in a DNA at an asymmetric recombinase target site, and which is not capable of recombining a DNA sequence of interest at an off-target recombinase target site in a DNA.

Surprisingly, it has been found that monomer-monomer interface mutations not only block the formation of homodimers, but also drastically reduce the recombination activity of heterodimers on asymmetric recombinase target sites, whereas mutations in the catalytic region of the first and the second recombinase enzyme led to the formation of heterodimers with high activity on asymmetric recombinase target sites.

Accordingly, in a preferred embodiment of the method of the invention, said first mutant recombinase enzyme and said second mutant recombinase enzyme of said obligate DNA recombinase obtained in step vii. each comprise at least one mutation in a catalytic site, which render said first recombinase enzyme and said second recombinase enzyme catalytically inactive when expressed in isolation.

More preferably, said first mutant recombinase enzyme and said second mutant recombinase enzyme of said obligate DNA recombinase obtained in step vii. do not contain monomer-monomer interface mutations.

Said first recombinase enzyme and said second recombinase enzyme according to steps ii. to vi. are preferably evolved by substrate linked directed evolution (SLiDE) or directed evolution. Recombinase evolution using substrate-linked protein evolution (SLiDE) is known in the art and e.g. described in Buchholz and Stewart, 2001; Sakara et al., 2007; Karpinski et al., 2016; and Lansing, et al., 2020 and in WO 2018/229226 A1, as described herein below.

In a preferred embodiment, the selection according to steps v. and vi. of the method of the invention iterates between the selection for obligate heterodimers that are catalytically active on the asymmetric target sites (positive selection), and between heterodimers that are not catalytically active on off-target sites, preferably symmetric target sites.

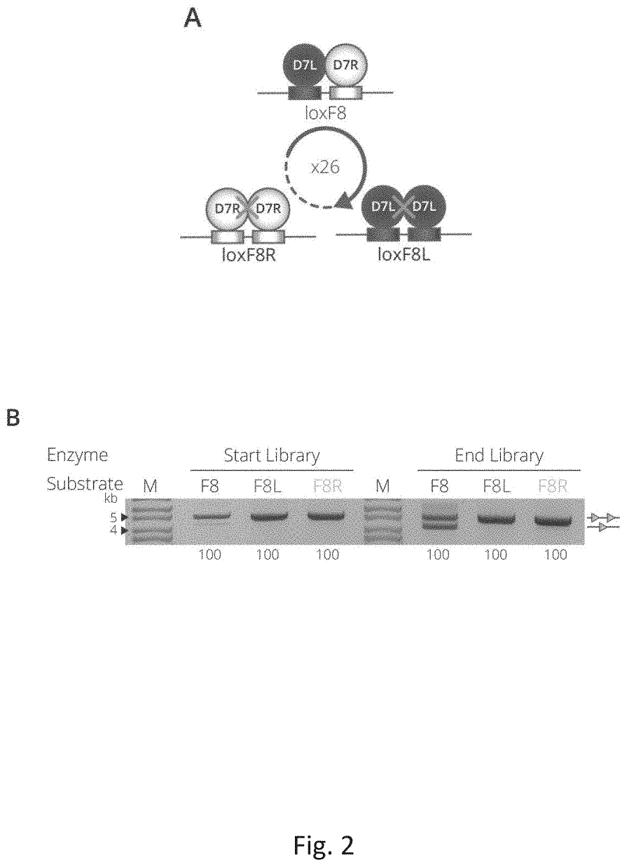

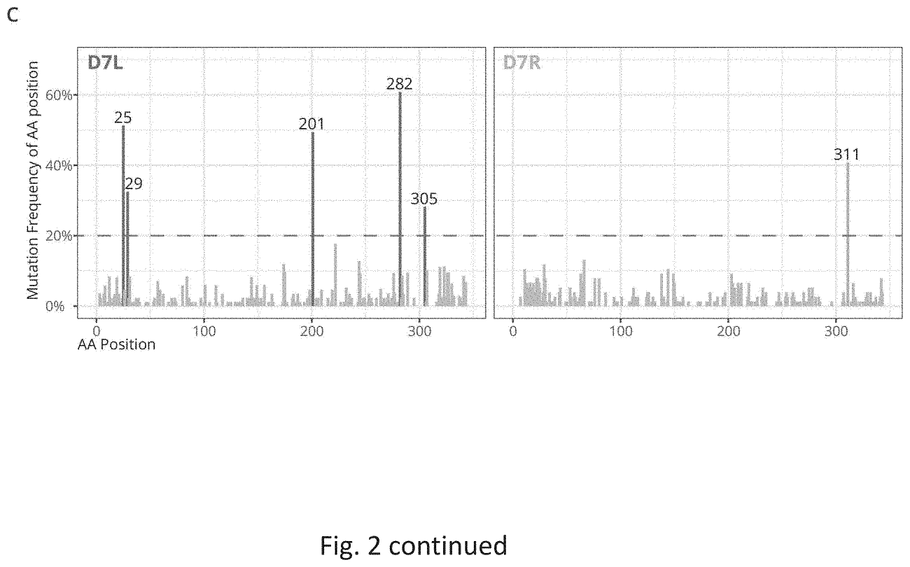

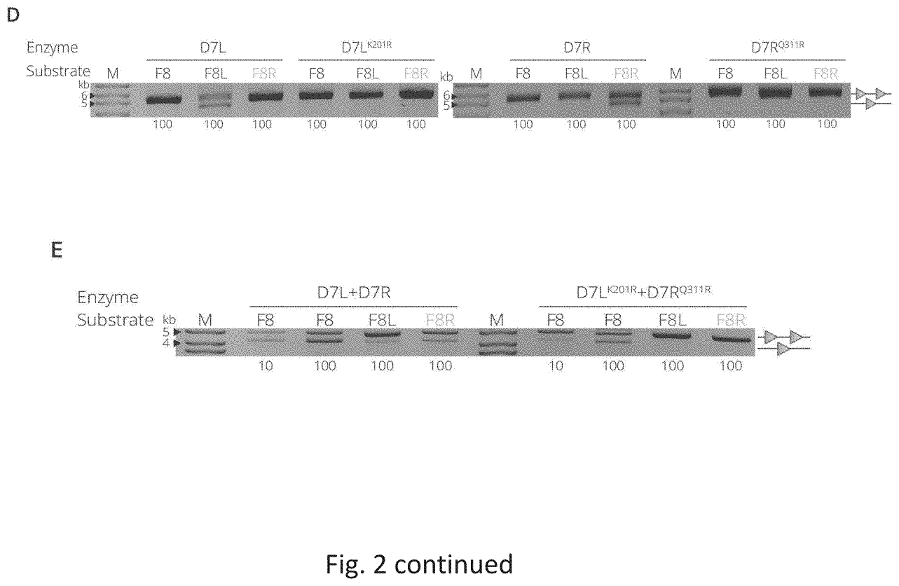

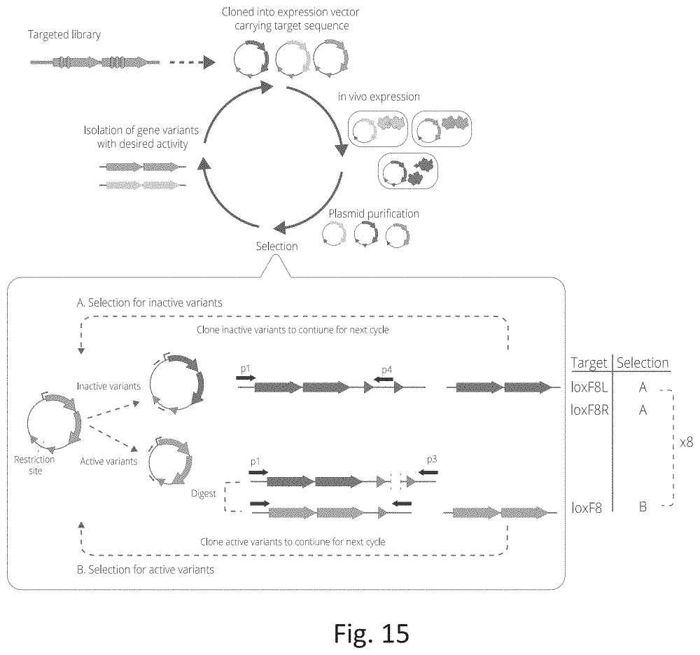

The method according to the invention comprises the generation of positive selection pressure for activity on the asymmetric target site and the generation of negative selection pressure on the symmetric target sites, wherein the generation of positive selection pressure and said negative selection pressure is achieved by the diversification of the two libraries of DNA recombining enzymes through error prone PCR (e.g. error-prone MyTaq DNA Polymerase, Bioline) and selection of the mutant pairs of the first recombinase enzyme and the second recombinase enzyme with activity on the desired asymmetric target site. This is exemplary described in more detail in working example 2 and in FIGS. 2 and 15.

Further preferably, at least 10, more preferably at least 15, most preferably at least 20 SLIDE cycles of positive and negative selection are performed.

In a further preferred embodiment of the method of the invention, the positive selection screen and the negative selection screen include the purification of the expression vectors after cultivation of the cells obtained in step iv. and the analysis for recombined and non-recombined vectors.

Most preferably, the positive selection screen is performed for the asymmetric target site loxF8, and wherein negative selection screen is performed for symmetric target sites loxF8R and loxF8L.

The method according to the invention may further comprise the steps of:
viii. identifying potential off-target sites of the desired obligate DNA recombinase;
ix. analyzing the recombinase activity of the desired obligate DNA recombinase on the off-target sites identified; and
x. selecting obligate DNA recombinases that do not show recombinase activity on at least one off-target site.

The invention further relates to obligate DNA recombinases that are identified or obtained with any of the afore described methods. The features, characteristics and embodiments described hereinabove for the obligate DNA recombinase apply equally to the method for generating obligate DNA recombinases for genome editing and the obligate DNA recombinases obtained by said method.

The genetically engineered DNA recombining enzyme of the invention or the nucleic acid molecule, the recombinant polynucleotide or the expression vector or the host cell of the invention can further be comprised in a pharmaceutical composition, which may optionally further contain one or more therapeutically acceptable diluents or carriers.

According to a further aspect, the present invention provides pharmaceutical compositions, e.g. for use in preventing or treating a disorder that can be treated by genome editing, such us hemophilia A. A pharmaceutical composition of the present invention comprises the at least one first DNA recombinase enzyme and/or the at least one second DNA recombinase enzyme of the invention or the DNA recombining enzyme of the invention, or the nucleic acid molecule or the plurality of nucleic acid molecules, or the polyrecombinant polynucleotide, or the expression vector, or the host cell of the invention, and one or more therapeutically acceptable diluents or carriers.

A pharmaceutical composition according to a preferred embodiment comprises a therapeutically effective amount of a vector which comprises a nucleic acid sequence of a polynucleotide that encodes one or more genetically engineered proteins according to the invention, or which comprises a therapeutically active amount of a nucleic acid encoding a genetically engineered DNA recombining enzyme of the invention, or which comprises a therapeutically active amount of a recombinant genetically engineered DNA recombining enzyme of the invention, or which comprises a therapeutically active amount of the host cell(s) of the invention (together named “therapeutically active agents”).

The present invention also provides methods of treating a disease or disorder and specifically a genetic disease or disorder by administering to a subject in need thereof a therapeutically effective amount of the genetically engineered DNA recombining enzyme, or the nucleic acid molecule, or the recombinant polynucleotide, or the expression vector, or the host cell of the invention.

It will be understood that the single dosage or the total daily dosage of the therapeutically active agents and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific nucleic acid or polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range per adult per day. The therapeutically effective amount of the therapeutically active agents, such as a vector according to the invention that should be administered, as well as the dosage for the treatment of a pathological condition with the number of viral or non-viral particles and/or pharmaceutical compositions described herein, will depend on numerous factors, including the age and condition of the patient, the severity of the disturbance or disorder, the method and frequency of administration and the particular peptide to be used.

The pharmaceutical compositions that contain a therapeutically active agent according to the invention may be in any form that is suitable for the selected mode of administration.

In one embodiment, a pharmaceutical composition of the present invention is administered parenterally.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

The therapeutically active agents of the invention can be administered, as sole active agent, or in combination with other active agents, in a unit administration form, as a mixture with conventional pharmaceutical supports, to animals and human beings.

In further embodiments, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and storage and must be preserved against the contaminating action of microorganisms, such as bacteria and fungi.

Solutions comprising the therapeutically active agents as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The therapeutically active agents can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be as solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions can be administered in a manner compatible with the dosage formulation and in such amount as is therapeutically effective. The formulations are easily administered in a variety of dosage forms, such as the type of injectable solutions described above, but drug release capsules and the like can also be employed. Multiple doses can also be administered. As appropriate, the therapeutically active agents described herein may be formulated in any suitable vehicle for delivery. For instance, they may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but are not limited to water, alcoholic/aqueous solutions, emulsions or suspensions, including saline and buffered media. Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes.

An appropriate therapeutic regimen can be determined by a physician, and will depend on the age, sex, weight, of the subject, and the stage of the disease. As an example, for delivery of a nucleic acid sequence encoding a genetically engineered DNA recombining enzyme of the invention using a viral expression vector, each unit dosage of the genetically engineered DNA recombining enzyme expressing vector may comprise 2.5 μl to 100 μl of a composition including a viral expression vector in a pharmaceutically acceptable fluid at a concentration ranging from 10¹¹ to 10¹⁶ viral genome per ml, for example.

The effective dosages and the dosage regimens for administering a genetically engineered DNA recombining enzyme of the invention or of its subunits in the form of a recombinant polypeptide depend on the disease or condition to be treated and may be determined by the persons skilled in the art. An exemplary, non-limiting range fora therapeutically effective amount of a genetically engineered DNA recombining enzyme of the present invention is about 0.1-10 mg/kg/body weight, such as about 0.1-5 mg/kg/body weight, for example about 0.1-2 mg/kg/body weight, such as about 0.1-1 mg/kg/body weight, for instance about 0.15, about 0.2, about 0.5, about 1, about 1.5 or about 2 mg/kg/body weight.

A physician or veterinarian having ordinary skill in the art may readily determine and prescribe the effective amount of the pharmaceutical composition required. For example, the physician or veterinarian could start doses of the therapeutically active agents of the invention employed in the pharmaceutical composition at levels lower than that required in order to achieve the desired therapeutic effect and gradually increase the dosage until the desired effect is achieved. In general, a suitable daily dose of a composition of the present invention will be that amount of the delivery system which is the lowest dose effective to produce a therapeutic effect. Such an effective dose will generally depend upon the factors described above. Administration may e.g. be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible for a delivery system of the present invention to be administered alone, it is preferable to administer the delivery system as a pharmaceutical composition as described above.

Further provided are kits comprising a therapeutically active agent as described above and herein. In one embodiment, the kit provides the therapeutically active agents prepared in one or more unitary dosage forms ready for administration to a subject, for example in a preloaded syringe or in an ampoule. In another embodiment, the therapeutically active agents are provided in a lyophilized form.

A nucleic acid sequence that is a potential target site for DNA-recombining enzymes that are capable to induce a site-specific DNA recombination of a sequence of interest in a genome can be identified according to method described in WO 2018/229226 A1, which comprises for example the sub-steps of:
a) Screening the genome or a part thereof comprising the. sequence of interest for two sequences that are potential spacer sequences, with a length of at least 5 and up to 12 bp, wherein one of the potential spacer sequence lies upstream of the sequence of interest and the other potential spacer sequence lies downstream of the sequence of interest and wherein the two sequences have a maximum distance of 2 megabases or less, preferably of 1.5 megabases or less or 1 megabases or less, more preferably of 900 kb, 800 kb, 700 kb, 600 kb or 500 kb, most preferably of 400 kb or 300 kb and a minimum distance of 150 bp,
b) Identifying potential target sites by determining for each potential spacer sequence the neighboring nucleotides, preferably 10 to 20 nucleotides, more preferably 12 to 15 nucleotides, most preferably 13 nucleotides on one side thereof form the potential first half site and the neighboring nucleotides, preferably 10 to 20 nucleotides, more preferably 12 to 15 nucleotides, most preferably 13 nucleotides, on the other side form the potential second half site, whereas both potential half sites and the spacer sequence in between form a potential target site,
c) The potential target sites identified in step b) are further screened to select for potential target sequences that do not occur (elsewhere) in the genome of the host to ensure a sequence specific recombination, preferably inversion.

Preferably, the sequences of the identified potential target sites for DNA-recombining enzymes are naturally occurring in the genome.

The first recombinase enzyme and the second recombinase enzyme can be evolved by directed evolution or rational design, preferably e.g. by substrate linked directed evolution (SLIDE) as described in WO 2018/229226 A1, wherein said directed evolution comprises the steps of:
a) Selecting a nucleotide sequence upstream of the nucleotide sequence to be altered as first target site and a nucleotide sequence downstream of the nucleotide sequence to be altered as second target site, whereas the sequences of the target sites are preferably not identical, wherein each target site comprises a first half site and a second half site with each 10 to 20 nucleotides separated by a spacer sequence with 5 to 12 nucleotides,
b) Applying molecular directed evolution on at least one library of DNA-recombining enzymes using a vector comprising the first target site and the second target site as selected in a) as substrate,
until at least one first designer DNA-recombining enzyme is obtained that is active on the first target site and at least one second designer DNA-recombining enzyme is obtained that is active on the second target site as selected in a).

Selecting an asymmetric target site provides the opportunity to compare two different evolution strategies. A single recombinase can be evolved to recognize both 10 to 20 bp, more preferably 12 to 15 bp, most preferably 13 bp half-sites or two recombinases can be evolved in parallel for each half-site. Combining the two recombinases allows to form a functional heterodimer capable of recombining the asymmetric site.

In one embodiment of the invention, it is preferred to evolve a single recombinase to recognize both 10 to 20 bp, more preferably 12 to 15 bp, most preferably 13 bp half-sites.

In a further embodiment of the invention, it is preferred to evolve two recombinases in parallel for each half-site resulting in a heterodimer. Because the heterodimer consists of two recombinases, which can form either a heterodimer or two different homodimers, the amount of potential recognition sequences is increased. This approach may disadvantageously result in the increased chance of unintended recombination at off-target sites. To reduce the chances of recombinations at off-target sites, it was a goal of the invention to constrain the monomers from homodimerization. To achieve this goal, the recombinase monomers were physically fused/bound to each other to enforce the desired heterodimer assembly, which is enabled by the mutations as disclosed herein.

In order to select genetically engineered DNA recombining enzymes that are highly specific for a desired recombinase target site, i.e. genetically engineered DNA recombining enzymes that show a reduced off-target recombination, the method of the invention may comprise in a further embodiment the steps of
i. Identifying potential off-target sites of the desired genetically engineered DNA recombining enzyme;
ii. Analyzing the recombinase activity of the desired genetically engineered DNA recombining enzyme on the off-target sites identified in step i.; and
iii. Selecting genetically engineered DNA recombining enzymes that do not show recombinase activity on at least one off-target site.

Recombinase off-target sites can for example be identified using bioinformatics approaches known to the person skilled in the art. Other approaches include ChIP-Seq-based assays to identify putative off-targets in the human followed by validation and DNA enrichment by qPCR. These methods are also known to the person skilled in the art.

Recombinase activity of a genetically engineered DNA recombining enzyme on these potential off-target sites can e.g. experimentally be tested by cloning the genomic sequences as excision substrates into a bacterial reporter vector, such as described herein below. Recombination at the off-target sites can then be detected by monitoring the expression of a reporter gene, e.g. using a PCR-based assay. Such an assay can also be performed in a human tissue culture to investigate whether an off-target site is altered by the genetically engineered DNA recombining enzyme in vivo.

Most preferably, the genetically engineered DNA recombining enzyme of the invention shows high specificity on the loxF8 target site with the target sequences of SEQ ID NOs: 65 and 66 and does not show activity on off-target sites at a high induction level. Off-target sites, which are preferably not recognized by the genetically engineered DNA recombining enzyme of the invention, are selected from the group consisting of SEQ ID NOs: 67 to 83 as shown in Table 4.

TABLE 4
Nucleic acid sequences of off-target sites

| Target | | SED |
|---|---|---|
| site | Sequence | ID NO: |
| Sym1 | ATAAATCTGTGGA AACGCTGC TCCACAGATTTAT | 67 |
| Sym2 | CTAAGATTGTGTG AACGCTGC CACACAATCTTAG | 68 |
| 1LR | ATAAATTTGTGGA AATTAAAC AACACACTCTTAA | 69 |
| 2LR | ACAAAATTGTGGA AATTAAAC AACACAATCTTAA | 70 |
| 3LR | ACAAATATGTGGA AATTAAAC AACACACTCTTGA | 71 |
| 4LR | ATAAATTTGTGGA AATTAAAC AACACACTCTTAA | 72 |
| 5LR | TTAAGAGTGTGTT TTTTAATT TCCACATATTTGT | 73 |
| HG1L | ATAAATATGTGAA TATACATA TTCACATATTTAT | 74 |
| HG2L | ATATATCTATAGA TATAGATA TCCACAGATATAT | 75 |
| HG1R | CTATGTTTTTGAG GTCTTATT CACAAAATCTTTT | 76 |
| HG2R | CTATTATTGTGTA ACAAATTA CCCCCAAACTTAG | 77 |
| HS4 | ATAAATATGTGTG TATATATA CACACAAACATAT | 78 |
| HS5 | ATATATCTGTGTA TATATATA CACACACACATAT | 79 |
| HS6 | ATATATATGTGTA TATATATA CACACATACATAT | 80 |
| HS7 | ATATATGTGTGTA TATATATA CACACACACATAC | 81 |
| HS8 | ATAAATATGTGTA AACTAAAC AACACACTCTTAA | 82 |
| HS9 | ACAAATATGTGGA AACTAAAC AACACATTCTTGA | 83 |

Bold: Sequences that are non-homologous to the target loxF8 sequence (SEQ ID NO: 65)
Underlined: Spacer sequences

It is a particular advantage of the invention that any recombinase target site can be used to evolve a recombinase enzyme for a genetically engineered DNA recombining enzyme that shows a specific activity for this recombinase target site. Since the method of the invention includes the provision of a target site specific obligate recombinase complex, wherein the monomers of recombinase heterodimers are specifically adapted by introducing single mutations in the evolved or naturally occurring recombinases. The method of the invention has further the advantage that undesired off-target-activity of the recombinase complex, i.e. the genetically engineered DNA recombining enzymes, can be drastically reduced, preferably completely eliminated. This makes the obligate recombinase complex, i.e. the genetically engineered DNA recombining enzymes especially suitable for use in gene therapies.

In a further embodiment, the invention provides a method for determining recombination on genomic level in a host cell culture, comprising a genetically engineered DNA recombining enzyme for efficient and specific genome editing according to the invention, wherein said method comprises the steps of:
i. providing a nucleic acid molecule encoding a first recombinase enzyme, wherein said first recombinase enzyme has been evolved by directed evolution or rational design to specifically recognize a first half-site of a recombinase target site and wherein said first recombinase enzyme comprises at least one mutation that inactivates the catalytic activity as a DNA recombinase of said first recombinase enzyme;
ii. providing a nucleic acid molecule encoding a second recombinase enzyme, wherein said second recombinase enzyme has been evolved by directed evolution or rational design to specifically recognize a second half-site of a recombinase target site and wherein said second recombinase enzyme comprises at least one mutation that inactivates the catalytic activity as a DNA recombinase of said second recombinase enzyme ;
iii. creating an expression vector by cloning the nucleic acid molecule encoding the first recombinase enzyme of step i. and the nucleic acid molecule encoding the second recombinase enzyme of step. ii. into an expression vector which further comprises a first reporter gene for expression of a first reporter protein;
iv. transfecting a host cell with the expression vector of step iii. and transfecting the host cell with a reporter plasmid comprising a second reporter gene for expressing a second reporter protein;
v. expressing the genetically engineered DNA recombining enzyme comprising said first recombinase enzyme and said second recombinase enzyme, wherein said genetically engineered DNA recombining enzyme is fused to the first reporter gene; and expressing the second reporter protein;
vi. identifying cells, which show a double expression of the first reporter protein and the second reporter protein, which is indicative for a successful recombination and thereby for the complementation of the function as DNA recombinase by forming an obligate genetically engineered DNA recombining enzyme comprising said first recombinase enzyme and said second recombinase enzyme.

A suitable first reporter gene according to step iii. is the gene encoding for EGFP. Consequently, a suitable first reporter protein is EGFP.

A suitable second reporter gene according to step iv. is the gene encoding for mCherry. Consequently, a suitable second reporter protein is mCherry.

This system has the advantage that the transfection efficiency of the cells transfected with both expression and reporter plasmid can be measured based on the GFP fluorescence. GFP and mCherry double positive cells reflect recombination of the reporter in human cells. In order to calculate the recombination efficiency of the reporter plasmid in human cells, the double positive cells can be normalized to the transfection efficiency.

Genetically engineered first recombinase enzymes and second recombinase enzymes and single mutations that inactivate their DNA recombinase activity, which are suitable for use in the method for determining recombination on genomic level in a host cell culture are described herein above. Likewise, suitable pairs of first recombinase enzymes and second recombinase enzymes that form obligate DNA recombinase complexes with a complemented DNA recombinase activity are described herein above as well.

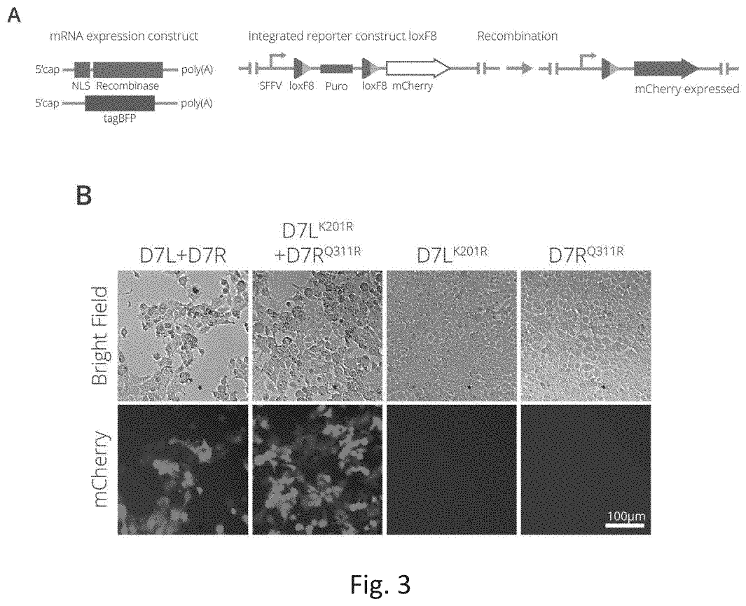

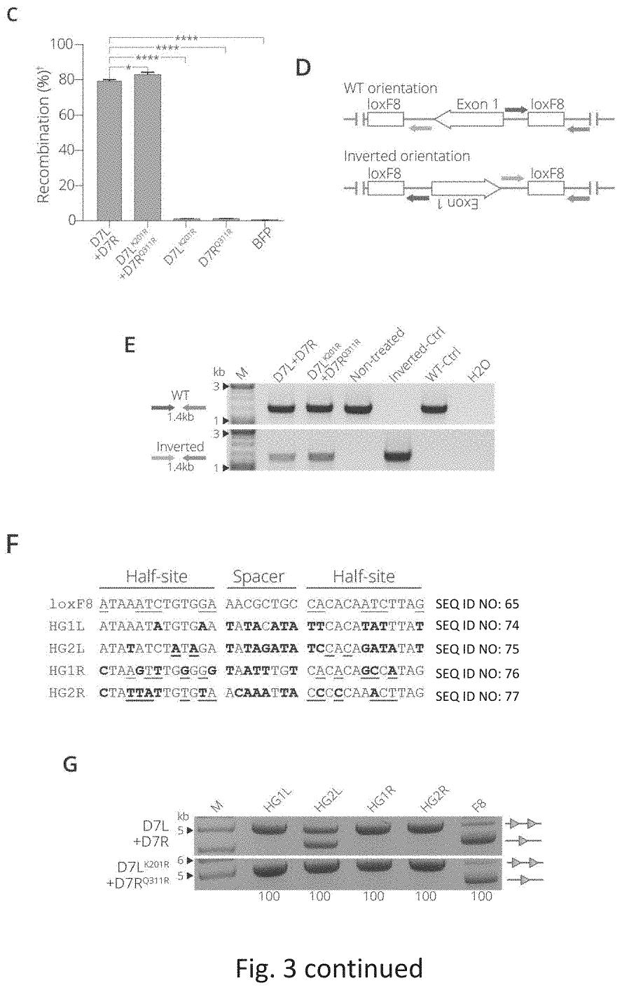

The genetically engineered DNA recombining enzymes described herein were, e.g. developed to correct a large gene inversion of exon 1 in the F8 gene which is causing Hemophilia A. To study the inversion efficacy of the heterodimers on genomic level, an in vitro recombinase assay as described in example 9 was developed. The inversion efficacy of the obligate heterodimer on genomic level in recombinase expressing cells was found to be equal to the non-obligate heterodimer. The same was demonstrated for the deletion efficiency of the obligate heterodimers according to the invention (see example 6 and FIG. 3, in particular FIG. 3C). The deletion efficiency of the obligate heterodimers according to the invention was even increased slightly. This is surprising, because conventional obligate heterodimers that have been produced by altering the protein-protein-interface, show a markedly decreased recombination activity.

Accordingly, the invention provides in a further embodiment a method for inversion of DNA sequence on genomic level in a cell, wherein said method comprises the steps of:
i. providing a nucleic acid molecule encoding a first recombinase enzyme, wherein said first recombinase enzyme comprises at least one mutation in its catalytic region that inactivates the catalytic activity as a DNA recombinase of said first recombinase enzyme and wherein said first recombinase monomer specifically recognizes a first half-site of a recombinase target site;
ii. providing a nucleic acid molecule encoding a second recombinase enzyme, wherein said second recombinase enzyme comprises at least one mutation in its catalytic region that inactivates the catalytic activity as a DNA recombinase of said second recombinase enzyme and wherein said second recombinase monomer specifically recognizes a second half-site of a recombinase target site;
iii. creating an expression vector by cloning the nucleic acid molecule encoding the first recombinase enzyme and the nucleic acid molecule encoding the second recombinase enzyme into an expression vector;
iv. delivering to a cell, which comprises a DNA sequence to be inverted, the expression vector of step iii);
v. expressing the first and second recombinase enzymes in said cell;
vi. inversion of a DNA sequence, which is to be inverted, on a chromosome in said cell with said first and second recombinase enzymes expressed in said cell.

According to a preferred embodiment, the cell is a human cell and the inversion takes place of a human chromosome in said cell. According to a particlualry preferred embodiment, the cell is not a human germ cell.

Preferably, the DNA recombining enzyme of step v. is a genetically engineered DNA recombining enzyme of the invention as described herein, and more preferably recognizes the first half-site and the second half-site of an upstream target site and a downstream target site of a recombinase, most preferably the upstream target site of SEQ ID NO: 65 and the downstream target site of SEQ ID NO: 66 of the loxF8 recombinase or a reverse complement sequence thereof.

According to one embodiment, the expression vector of step iii) is delivered to said cell, e.g. by transfection.

According to an alternative embodiment, the method does not include a step of creating an expression vector, but comprises delivering to said cell an RNA molecule encoding a genetically engineered DNA recombining enzyme according to the invention.

In one embodiment, the method for inversion of DNA sequence on genomic level is performed in genetically engineered host cell.

In a preferred embodiment, the method for inversion of DNA sequence on genomic level is performed in vitro in a human cell derived from a patient, more preferably from a patient suffering from hemophilia A.

In a further preferred embodiment, the method for inversion of DNA sequence on genomic level is performed in patients, in particular in a patient suffering from hemophilia A in vivo.

Genetically engineered first recombinase enzymes and second recombinase enzymes as well as single mutations that inactivate their DNA recombinase activity, which are suitable for use in the method for inversion of DNA sequence on genomic level in a host cell culture are described herein above. Likewise, suitable pairs of first recombinase enzymes and second recombinase enzymes that form obligate DNA recombinase complexes with a complemented DNA recombinase activity are described herein above as well.

According to a further aspect, the present invention provides a method for treating or preventing a disease, comprising administering to a subject in need thereof the genetically engineered DNA recombining enzyme, or the nucleic acid molecule or the plurality of nucleic acid molecules, or the expression vector, or the host cell, or the pharmaceutical composition of the invention.

The present invention further provides a method for treating or preventing hemophilia A, comprising administering to a subject in need thereof the genetically engineered DNA recombining enzyme , or the nucleic acid molecule or the plurality of nucleic acid molecules, or the expression vector, or the host cell, or the pharmaceutical composition of the invention. Optionally, the hemophilia A is severe hemophilia A.

The present invention further provides a method for recombination of a target DNA sequence in a cell, comprising introducing into the cell:
(a) a nucleic acid molecule encoding the first recombinase enzyme and a nucleic acid molecule encoding the second recombinase enzyme of the invention; and/or
(b) the first recombinase enzyme and the second recombinase enzyme of the invention, thereby recombining the target DNA sequence in the cell.

According to one embodiment, the nucleic acid molecule encoding the first recombinase enzyme and/or the nucleic acid molecule encoding the second recombinase enzyme is an mRNA.

According to a further embodiment, the nucleic acid molecule encoding the first recombinase enzyme and/or the nucleic acid molecule encoding the second recombinase enzyme is an expression vector, preferably in an expression vector as described herein.

The present invention also pertains to the following items:

Item 1.A genetically engineered DNA recombining enzyme comprising a complex of at least a first DNA recombinase enzyme and at least a second DNA recombinase enzyme, wherein said first DNA recombinase enzyme and said second DNA recombinase enzyme specifically recognize a first half-site and a second half-site of an upstream target site and/or a downstream target site of a DNA recombinase, wherein said first DNA recombinase enzyme and said second DNA recombinase enzyme each comprises a single amino acid substitution in its catalytic region, wherein said first DNA recombinase enzyme and said second DNA recombinase enzyme carrying said single amino acid substitution when expressed in isolation do not show the catalytic activity of a DNA recombinase, and wherein said first DNA recombinase enzyme and said second DNA recombinase enzyme carrying said single amino acid substitution when co-expressed and forming a complex show the catalytic activity of a DNA recombinase. Preferably, the single amino acid substitution in the catalytic region of the first recombinase enzyme is different from the single amino acid substitution in the catalytic region of the second recombinase enzyme. More preferably, the single amino acid substitution in the catalytic region of the first recombinase enzyme is at a different position in the catalytic region than the single amino acid substitution in the catalytic region of the second recombinase enzyme.

Item 2.The DNA recombining enzyme according to item 1, wherein the at least one first recombinase and the at least one second recombinase are of the same type.

Item 3.The DNA recombining enzyme according to item 2, wherein the at least one first recombinase and the at least one second recombinase are both Cre-, Dre-, VCre-, SCre-, Vika-, lambda-Int-, Flp-, R-, Kw-, Kd-, B2-, B3-, Nigri- or Panto-recombinases.

Item 4.The DNA recombining enzyme according to any one of items 1 to 3, wherein the DNA recombining enzyme is a complex of recombinases in form of a heterotetramer.

Item 5.The DNA recombining enzyme according to any one of the preceding items, wherein the single amino acid substitution in the at least one first and in the at least one second recombinase enzyme is at a position of a conserved amino acid in the catalytic region.

Item 6.The DNA recombining enzyme according to any one of the preceding items, wherein the single amino acid substitution in the at least one first and in the at least one second recombinase enzyme is selected from the group consisting of: E129R, Q133H, R173A, R173C, R173D, R173E, R173F, R173G, R173I, R173K, R173L, R173M, R173N, R173P, R173Q, R173S, R173T, R173V, R173W, R173Y, E176H, E176I, E176L, E176M, E176V, E176W, E176Y, K201A, K201C, K201C, K201D, K201F, K201G, K201H, K201I, K201L, K201M, K201N, K201P, K201Q, K201R, K201S, K201T, K201V, K201W, K201Y, H289D, H289E, H289I, H289K, H289R, H289W, R292A, R292C, R292E, R292F, R292G, R292H, R292I, R292L, R292M, R292N, R292P, R2920, R292S, R292T, R292V, R292W, R292Y, Q311R, W315C, W315E, W315G, W315I, W315K, W315L, W315M, W315N, W315Q, W315R, W315S, W315T, W315V, Y324A, Y324C, Y324E, Y324F, Y324H, Y324I, Y324K, Y324L, Y324M, Y324N, Y3240, Y324R, Y324S, Y324T, Y324V, and Y324W of SEQ ID NO: 1, or in a corresponding position of another recombinase, preferably in a corresponding position of SEQ ID NO: 14, SEQ ID NO: 17 or SEQ ID NO: 20.

Item 7.The DNA recombining enzyme according to any one of the preceding items, wherein the single amino acid substitution in the at least one first and in the at least one second recombinase enzyme is at a position selected from the group consisting of:
(i) E129, Q133, R173, E176, K201, H289, R292, Q311, W315, and Y324 of SEQ ID NO: 1;
(ii) E146, Q151, R191, N194, K219, H308, R311, Q330, W334, and Y343 of SEQ ID NO: 14;
(iii) E130, Q134, R174, E177, K202, H290, R293, Q312, W316, and Y325 of SEQ ID NO: 17;
(iv) E131, Q135, R175, E178, K202, H290, R293, Q312, W316, and Y325 of SEQ ID NO: 20; or
(v) at an amino acid position in another recombinase, wherein said amino acid position in the other recombinase corresponding to position E129, Q133, R173, E176, K201, H289, R292, Q311, W315, or Y324 of SEQ ID NO: 1.

Item 8.A genetically engineered DNA recombining enzyme comprising a complex of at least a first DNA recombinase enzyme and at least a second DNA recombinase enzyme, wherein
(i) the first recombinase enzyme is a polypeptide having an amino acid sequence with at least 70%, preferably 80%, more preferably 90%, sequence identity with a sequence according to SEQ ID NO: 7 and comprises the single mutation K201R in its catalytic region; and wherein the second recombinase enzyme is a polypeptide having an amino acid sequence with at least 70%, preferably 80%, more preferably 90%, sequence identity with a sequence according to SEQ ID NO: 11 and comprises the single mutation Q311R in its catalytic region; or
(ii) the first recombinase enzyme is a polypeptide which has an amino acid sequence having at least 70%, preferably 80%, more preferably 90%, sequence identity with a sequence according to SEQ ID NO: 7 and comprises the single mutation K201R in its catalytic region; and wherein the second recombinase enzyme is a polypeptide having an amino acid sequence with at least 70%, preferably 80%, more preferably 90%, sequence identity with a sequence according to SEQ ID NO: 12 and comprises the single mutation Q311K in its catalytic region.

Item 9.A DNA recombining enzyme comprising:
(i) a first Cre recombinase comprising the single mutation K201R in the catalytic region and having an amino acid sequence which is at least 70%, preferably 80%, more preferably 90%, identical with a polypeptide having the sequence according to SEQ ID NO: 2; and a second Cre recombinase comprising the single mutation Q311R in the catalytic region and having an amino acid sequence which is at least 70%, preferably 80%, more preferably 90%, identical with a polypeptide having the sequence according to SEQ ID NO: 5;
(ii) a first Cre recombinase comprising the single mutation K201R in the catalytic region and having an amino acid sequence which is at least 70%, preferably 80%, more preferably 90%, identical with a polypeptide having the sequence according to SEQ ID NO: 2; and a second Cre recombinase comprising the single mutation Q311K in the catalytic region and having an amino acid sequence which is at least 70%, preferably 80%, more preferably 90%, identical with a polypeptide having the sequence according to SEQ ID NO: 4;
(iii) a first Vika recombinase comprising the single mutation K219R in the catalytic region and having an amino acid sequence which is at least 70%, preferably 80%, more preferably 90%, identical with a polypeptide having the sequence according to SEQ ID NO: 15; and a second Vika recombinase comprising the single mutation Q330R in the catalytic region and having an amino acid sequence which is at least 70%, preferably 80%, more preferably 90%, identical with a polypeptide having the sequence according to SEQ ID NO: 16;
(iv) a first Panto recombinase comprising the single mutation K202R in the catalytic region and having an amino acid sequence which is at least 70%, preferably 80%, more preferably 90%, identical with a polypeptide having the sequence according to SEQ ID NO: 18; and a second Panto recombinase comprising the single mutation Q312R in the catalytic region and having an amino acid sequence which is at least 70%, preferably 80%, more preferably 90%, identical with a polypeptide having the sequence according to SEQ ID NO: 19;
(v) a first Dre recombinase comprising the single mutation K202R in the catalytic region and having an amino acid sequence which is at least 70%, preferably 80%, more preferably 90%, identical with a polypeptide having the sequence according to SEQ ID NO: 21; and a second Dre recombinase comprising the single mutation Q312R in the catalytic region and having an amino acid sequence which is at least 70%, preferably 80%, more preferably 90%, identical with a polypeptide having the sequence according to SEQ ID NO: 22; or
(vi) a first Vcre recombinase comprising the single mutation K221R in the catalytic region and having an amino acid sequence which is at least 70%, preferably 80%, more preferably 90%, identical with a polypeptide having the sequence according to SEQ ID NO: 24; and a second Vcre recombinase comprising the single mutation Q336R in the catalytic region and having an amino acid sequence which is at least 70%, preferably 80%, more preferably 90%, identical with a polypeptide having the sequence according to SEQ ID NO: 25.

Item 10. A nucleic acid molecule encoding the at least one first DNA recombinase enzyme and/or the at least one second DNA recombinase enzyme according to any one of items 1 to 9.

Item 11. An expression vector comprising the nucleic acid molecule according to item 10 and one or more expression-controlling elements operably linked with said nucleic acid to drive expression thereof.

Item 12. A host cell comprising the nucleic acid molecule according to item 10 or the expression vector according to item 11.

Item 13. A pharmaceutical composition comprising the at least one first DNA recombinase enzyme and/or the at least one second DNA recombinase enzyme according to any one of items 1 to 9, or the nucleic acid molecule according to item 10, or the expression vector according to item 11, or the host cell according to item 12, and one or more therapeutically acceptable diluents or carriers.

Item 14. The complex of DNA recombinases according to any one of items 1 to 9, or the nucleic acid molecule according to item 10, or the expression vector according to item 11, or the host cell according to item 12, or the pharmaceutical composition according to item 13, for use in medicine.

Item 15. The complex of DNA recombinases according to any one of items 1 to 9, or the nucleic acid molecule according to item 10, or the expression vector according to item 11, or the host cell according to item 12, or the pharmaceutical composition according to item 13, for use in the treatment of hemophilia A, preferably for use in treating severe hemophilia A.

Item 16. A method for generating obligate DNA recombinases for genome editing, wherein said method comprises the steps of:
(i) providing a nucleic acid molecule encoding a first recombinase enzyme and a nucleic acid molecule encoding a second recombinase enzyme, wherein said first recombinase enzyme binds to a first half site of an asymmetric recombinase target site and said second recombinase enzyme binds to a second half site of an asymmetric recombinase target site, wherein said first recombinase enzyme and said second recombinase enzyme form a heterodimer which is capable of inducing a site-specific DNA recombination of a sequence of interest at said asymmetric recombinase target site in a DNA sequence, wherein said asymmetric recombinase target site comprises a first half site and a second half site of an upstream target site and/or a downstream target site of a DNA recombinase an upstream and a downstream half site which wherein said first half site and a second half site are not identical and which are not palindromic;
(ii) mutagenesis to create libraries of nucleic acid molecules encoding mutant first recombinase enzymes and of nucleic acid molecules encoding mutant second recombinase enzymes, wherein mutations are introduced in said first recombinase enzyme and said second recombinase enzyme;
(iii) creating expression vectors, by cloning the library of the nucleic acid molecules encoding a first mutant recombinase enzyme and the library of nucleic acid molecules encoding a second mutant recombinase enzyme into expression vectors, wherein said expression vectors carry a DNA sequence of interest, which is to be recombined;
(iv) transfecting a cell with the expression vectors of step iii) and expressing the libraries of said mutant first recombinase enzyme and said mutant second recombinase enzyme in the same cell resulting in the formation of recombinase heterodimers comprising a mutant first recombinase enzyme and a mutant second recombinase enzyme;
(v) performing positive selection screens for heterodimers obtained in step iv. that are capable to induce a site-specific DNA recombination of a sequence of interest at an asymmetric recombinase target site in a DNA;
(vi) performing negative selection screens for heterodimers obtained in step iv. or v. that are not capable to induce a site-specific DNA recombination of a sequence of interest at an off-target, preferably symmetric recombinase target site in a DNA;
(vii) selecting an obligate DNA recombinase which is capable of recombining a DNA sequence of interest at a recombinase target site in a DNA comprising a first half site and a second half site of an upstream target site and/or a downstream target site of a DNA recombinase, and which is not capable of recombining a DNA sequence of interest at an off-target, preferably symmetric recombinase target site in a DNA;
wherein in said obligate DNA recombinase obtained in step (vii), said first mutant recombinase enzyme and said second mutant recombinase enzyme each comprises at least one mutation in a catalytic site which renders said first recombinase enzyme and said second recombinase enzyme catalytically inactive when expressed in isolation.

Item 17. A method for generating a complex of obligate DNA recombinase enzymes, said method comprising the steps of:

(i) introducing a single amino acid substitution into the catalytic region of a first DNA recombinase enzyme, wherein said single amino acid substitution renders the first DNA recombinase enzyme catalytically inactive;

(ii) introducing a single amino acid substitution into the catalytic region of a second DNA recombinase enzyme, wherein said single amino acid substitution renders the second DNA recombinase enzyme catalytically inactive;

(iii) co-expressing both the mutated first and the mutated second DNA recombinase enzymes in a host cell;

(iv) isolating the mutated first and the mutated second DNA recombinase enzymes from the host cell.

Item 18. A DNA recombining enzyme obtained by the method of item 16 or 17.

Item 19. An in vitro method for inversion of a DNA sequence on genomic level in a cell, comprising the steps of:
(i) providing a nucleic acid molecule encoding a first recombinase enzyme as described in any one of items 1 to 9, wherein said first recombinase enzyme specifically recognizes a first half-site of a recombinase target site;
(ii) providing a nucleic acid molecule encoding a second recombinase enzyme as described in any one of items 1 to 9, wherein said second recombinase enzyme specifically recognizes a second half-site of a recombinase target site;
(iii) creating an expression vector by cloning the nucleic acid molecule encoding the first recombinase enzyme and the nucleic acid molecule encoding the second recombinase enzyme into an expression vector;
(iv) delivering said expression vector to a cell, which comprises a DNA sequence to be inverted;
(v) expressing the first recombinase enzyme and the second recombinase enzyme in said cell;
(vi) allowing formation of a DNA recombinase complex comprising the first recombinase enzyme and the second recombinase enzyme; and
(vii) allowing inversion of the DNA sequence to be inverted in said cell.

### EXAMPLES OF THE INVENTION

The following examples are provided for the sole purpose of illustrating various embodiments of the present invention and are not meant to limit the present invention in any fashion.

### Example 1: Plasmid Construction

Previously described plasmids containing the target sites of loxF8, loxF8L and loxF8R were used for evolution (pEVO-loxF8, pEVO-loxF8L and pEVO-loxF8R respectively) (10). The mutations published by Zhang et al. (16) were introduced into the sequence of both D7 subunits through DNA fragment synthesis (Twist Bioscience) with mutations A3 (K25R, D29R, R32E, D33L, G35R, R337E, E123L) applied to D7L and mutations 132 (E69D, R72E, L76E, E308R) applied to D7R. The synthesized fragments were inserted into the pEVO vectors in two cloning steps. First D7L^{A3} with Sad and Xhol and then D7R⁸² with BsrGl and Xbal (NEB). Once the correct sequences were confirmed by Sanger sequencing with primers 1 and 2 (Table 5) both molecules were subcloned into the three different pEVO vectors containing the target sites of loxF8, loxF8L and loxF8R with the restriction enzymes Sad and Sbfl (NEB).

TABLE 5
Cloning Primers

| Primer | SEQ ID | | | |
|---|---|---|---|---|
| number | NO | Oligo Name | Description | Oligo Sequence (5′-3′) |
| 1 | 85 | Rec-pEVO-F | Sequence validation of | TGCATCAGACATTGCCGTCA |
| | | | recombinase in pEVO | |
| 2 | 86 | Rec-pEVO-R | Sequence validation of | AGACCGCTTCTGCGTTCTGA |
| | | | recombinase in pEVO | |
| 3 | 87 | Positive | Selection of recombinases active | AAGGGAATAAGGGCGACACG |
| | | Selection R | on loxF8 (binds downstream of | |
| | | | target sites) | |
| 4 | 88 | Negative | Selection of recombinases | CTAACTGACACACATTCCACA |
| | | selection R | inacvite on the loxF8L or | |
| | | | loxF8R sites | |
| 5 | 89 | ISOR_D7R76_R | degenerate VNS codon D7R | ACGCGTCTGAAGGTGSNBGAGGTAAT |
| | | | Position 76 (SNB) | C |
| 6 | 90 | ISOR_D7L_32_ | degenerate VNS codon D7L | AGAAAACGCCCGGCGSNBSNBGAAGA |
| | | 33_R | Position 32 and 33 (SNB) | C |
| 7 | 1 | ISOR_D7L_25_ | Degenerate VNS codon D7L | AGTGATGAGGCTCGCVNSAACCTGAT |
| | | 29_F_VNS | position 25 and 29 | GVNSGTCTTC |
| 8 | 92 | ISOR_D7R_69_ | Degenerate VNS codon D7R | GTAGAACCTVNSGATGTTVNSGATTAC |
| | | 72_F_VNS | position 69 and 72 | CTC |
| 9 | 93 | ISOR_D7L_F | Forward nested PCR primer for | ATGTCCAATCTACAGACCCTACACCAG |
| | | | D7L | AATTTG |
| 10 | 94 | ISOR_D7L_25_ | Degenerate GHW codon D7L | GAAGACWDCCATCAGGTTWDCGCGA |
| | | 29_R_GHW | position 25 and 29 | GCCTCATCACT |
| 11 | 95 | ISOR_D7L_25_ | Degenerate MDG codon D7L | GAAGACCHKCATCAGGTTCHKGCGAG |
| | | 29_R_MDG | position 25 and 29 | CCTCATCACT |
| 12 | 96 | ISOR_D7L_32_ | Degenerate GHW codon D7L | GTCTTCGHWGHWCGCGHWGCGTTTT |
| | | 33_35_F_GHW | position 32, 33 and 35 | CTGAAGCT |
| 13 | 97 | ISOR_D7L_32_ | Degenerate MDG codon D7L | GTCTTCMDGMDGCGCMDGGCGTTTT |
| | | 33_35_F_MDG | position 32, 33 and 35 | CTGAAGCT |
| 14 | 98 | ISOR_D7L_R | Reverse nested PCR primer for | ATTCAGCTTGCACCATGCCGCCCACGT |
| | | | D7L | CCGGCA |
| 15 | 99 | ISOR_D7R_F | Forward nested PCR primer for | CTGTCCGTTTGCCGGTCGTGGGCGGCA |
| | | | D7R | TGGTGC |
| 16 | 100 | ISOR_D7R_69_ | Degenerate GHW codon D7R | GAGGTAATCWDCAACATCWDCAGGT |
| | | 72_R_GHW | position 69 and 72 | TCTACGGG |
| 17 | 101 | ISOR_D7R_69_ | Degenerate MDG codon D7R | GAGGTAATCCHKAACATCCHKAGGTTC |
| | | 72_R_MDG | position 69 and 72 | TACGGG |
| 18 | 102 | ISOR_D7R_76_ | Degenerate GHW codon D7R | GATTACCTCGHWCACCTTCAGACGCG |
| | | F_GHW | position 76 | TGGTCTG |
| 19 | 103 | ISOR_D7R_76_ | Degenerate MDG codon D7R | GATTACCTCMDGCACCTTCAGACGCG |
| | | F_MDG | position 76 | TGGTCTG |
| 20 | 104 | ISOR_D7R_R | Reverse nested PCR primer for | AGCCCGACGGTGAAGCATGTTTAGCG |
| | | | D7R | AGCCCAG |
| 21 | 105 | FWD | mRNA production adding T7 | GCTAATACGACTCACTATAGGGAGAG |
| | | mRNA for Cre: | promoter | CCGCCACCATGCCAAAAAAGAAGAGA |
| | | T7 promoter- | | AAGGTAATGTCCAATTTACTGACCGTA |
| | | Kozak | | CACCA |
| | | seq-SV40 | | |
| | | NLS-Cre | | |
| 22 | 106 | REV | mRNA production for Cre adding | TTTTTTTTTTTTTTTTGGTTTATTCCTAA |
| | | mRNA for Cre: | poly(A) signal | TCGCCATCTTCCAGCAG |
| | | poly(A) | | |
| | | signal-Cre | | |
| 23 | 107 | FWD_TS | forward primer to amplify over | TGTATCCGCTCATGAGACAA |
| | | | target sequences in pEVO | |
| 24 | 108 | REV_TS | reverse primer to amplify over | TTAAACGCCTGGTTGCTAC |
| | | | target sequences in pEVO | |

The vector used for library analysis, pEVO-lacZ, was adapted from a previously described selection plasmid (8). Symmetric target sites loxF8L (top strand 5′-3′ ATAAATCTGTGGAGCATACATTCCACAGATTTAT, SEQ ID NO: 65) and loxF8R (top strand 5′-3′ CTAAGATTGTGTGGCATACATCACACAATCTTAG, SE Q ID NO: 66) were added with the spacer sequence of loxP to prevent the recombination of the symmetric sites with the loxF8 site. The symmetric target sites flank two strong transcriptional terminators (17). Upon recombination, the removal of the terminator sequences allows for the transcription of the lacZa fragment which is driven by the constitutive cat promoter.

### Example 2: Substrate-Linked Directed Evolution

Recombinases were evolved using the previously described substrate-linked protein evolution (SLiDE) (Buchholz and Stewart, 2001; Sakara et al., 2007; Karpinski et al., 2016; Lansing, et al., 2019). By varying selection of active and inactive recombinases on the loxF8 and symmetric sites, respectively, a counterselection strategy was established.

Positive selection pressure for activity on the asymmetric site (loxF8) and negative selection pressure on the symmetric sites (loxF8L and loxF8R) were achieved through a modified method of substrate-linked directed evolution (6). Each cycle of evolution involved the diversification of the libraries through error prone PCR (MyTaq DNA Polymerase Bioline) and selection of the variants for the desired activity on the target site. The diversified libraries were cloned into the pEVO containing the target site, then the vector was transformed into electrocompetent XL1-Blue E. coli to express the recombinase variants overnight via an Arabinose inducible promoter. Selection cycled between the positive and negative selection strategies. To perform positive selection for loxF8 recombination, the purified plasmid was digested with enzymes Ndel and Avrll to linearize all non-recombined variants, and was then amplified with primers 1 and 3 (Table 5). Negative selection was achieved by having a primer that could bind between the symmetric target sites (primer 4, Table 5) amplifying only those recombinases that have not carried out a recombination event. For each round of evolution, selection was alternated between the three target sites. Recombination efficiency was monitored through the plasmid-based activity assay. A scheme of the evolution method applied is shown in FIG. 15.

### Example 3: Recombinase Activity Assay: Plasmid-Based and Blue-White Screen

To visualize the recombination activity of the recombinase or recombinase library on the target site of interest a plasmid-based assay was used as previously described (6, 9, 10).

For activity analysis and selection of active variants of the final library, a blue-white activity screen was used. The library was cloned to the pEVO LacZ counter selection plasmid with restriction enzymes Sacl and Sbfl. Once plated on Xgal indicator plates containing antibiotic selection and arabinose, the activity of the recombinase variant was read as blue or white. White colonies either represent an inactive recombinase pair or a pair that is highly specific for loxF8. To eliminate the inactive recombinases, the library was induced over night with a low (10 μg/ml L-Arabinose, Sigma) arabinose level prior to the blue-white screening. The purified plasmid was digested with Ndel and Avrll to linearize non recombined plasmids then retransformed and induced with a higher level of arabinose (100 μg/ml L-Arabinose, Sigma) to allow for sensitive detection of low-level symmetric site activity. The blue colonies contained mutants that were active on the symmetric site. Therefore, the white colonies were selected, which contained mutants that did not recombine the symmetric site but recombined the LoxF8 site. 80 white colonies were selected and a colony PCR showed that 75 out of the 80 selected colonies had a desired activity profile showing a 1.7KB band.

### Example 4: Library Design

A3 residue positions K25, D29, R32 and D33 and B2 positions E69, R72 and L76 were targeted by ISOR (incorporating synthetic oligonucleotides via gene reassembly). To target the diversity to the A3 and B2 positions, an adapted method of incorporating synthetic oligonucleotides via gene reassembly (ISOR) was applied (18). Incorporated oligonucleotides were designed with the degenerate codon (VNS, GHW and MDG). VNS contains 16 possible amino acids (D, E, H, I, K, M, N, Q, S, A, G, L, P, T, V, R), GHW includes codons corresponding to 4 possible amino acid variants (D, E, A, V) and MDG includes codons corresponding to 5 amino acid variants (K, L, M, Q, R). The incorporated A3 and B2 oligonucleotides (primers 5-20 Table 3) were applied in parallel to the shuffled D7L and D7R recombinases respectively.

### Example 5: Sequencing Analysis

To determine which mutations were occurring at the highest frequency among the mutated recombinases, the amino acid sequences of the D7L mutants were aligned to the original D7L recombinase sequence and the D7R mutants were aligned to the original D7R recombinase sequence. From the alignments, the amount of mutations occurring at each position were divided by the total amount of samples sequenced to determine the mutational frequency at each position (FIG. 2C). The most commonly mutated residue positions for D7L are (highlighted in dark grey) K25, D29, K201, and S305 and for D7 the most commonly mutated residue is Q311. Residue positions 1-10 for the D7R mutants were removed because of the background due to poor reads. The primer binding site between the two recombinase libraries is not long enough to extract a proper read while keeping the mutated pairs together. It is necessary to apply a mutation to each monomer to control the complex formation. Without both monomers having their own unique mutation, D7L or D7R could still form a functional homotetramer, resulting in unwanted recombination of their correlating evolved half-site. Therefore, a combination of mutations was considered necessary.

### Example 6: Expression in Mammalian Cells and Recombination Efficiency in Mammalian Cells

A fluorescence-based reporter assay was used to determine the recombination properties of the obligate monomers as described previously (10). HEK293T cells were seeded at a density of 350, 000 cells/ml the day before transfection. mRNA encoding the obligate monomer and a blue fluorescent protein (BFP) was transfected into a HEK293 reporter cell line containing integrated lox sites flanking repeated SVpoly(A) sequences. Upon recombination of these sites, a downstream monomeric red fluorescent protein (mCherry) is expressed. The recombinase activity was quantified via FACS using the MACSQuant VYB flow cytometer (Miltenyi Biotec) 48 hours after transfection. The percent of recombination was determined by the percentage of cells displaying red fluorescence within the blue fluorescence population.

### Example 7: HEK293T Cell Culture

HEK293T cells were cultured using DMEM (Gibco) supplemented with 10% FBS (Capricon Scientific) and 1% Penicillin-Streptomycin (ThermoFisher) in a 12-well format. When reaching a confluency of 90%, the cells were split. Each well was washed once with PBS and 100 μl of Trypsin (Gibco) was added. After incubation for 3 min at 37° C., the detached cells were collected in a 15 ml tube. Cells were counted with the Countess 3 FL Automated Cell Counter (ThermoFisher) and seeded at a density of 75, 000 cells/well in 1 ml medium. For transfection the cells were seeded at a density of 350, 000 cells/well in 1 ml medium.

### Example 8: mRNA Transfection

HEK293T cells were transfected 24 h after seeding. For each transfection reaction, a 1.5 ml tube was prepared with a total of 300 ng of mRNA (100 ng tagBFP mRNA and 200 ng recombinase mRNA). 100 μl Opti-MEM I Reduced Serum Media was mixed with 1.5 μl Lipofectamine MessangerMax (ThermoFisher) and added to the mRNA sample. The mixture was shortly vortexed and incubated 15 min at RT. In the meantime, the medium of the cells was replaced with fresh medium. The transfection mixture was then added to the cells. The medium was changed on the following day and the cells were analyzed two days post transfection.

### Example 9: PCR-Based Genomic Inversion Detection

The inversion of the loxF8 locus after treating HEK293T cells with the D7 recombinase dimer was detected as described previously (10).

### Example 10: In Vitro Transcription (IVT)

mRNA was produced using the HiScribeTM T7 ARCA mRNA Kit (NEB) and purified with the Monarch RNA Cleanup Kit (NEB) following the manufacture's manual. The D7 recombinase dimer and tagBFP templates for the IVT were generated as previously described (10). The template for the different Cre variants was generated using primer 21 and primer 22 (Table 5). mRNA aliquots of 4 μg were stored at −80° C. for up to 6 months.

### Example 11: Evaluating Single Point Mutations in the Catalytic Region of the Recombination Synapses to Create the Obligate Phenotype

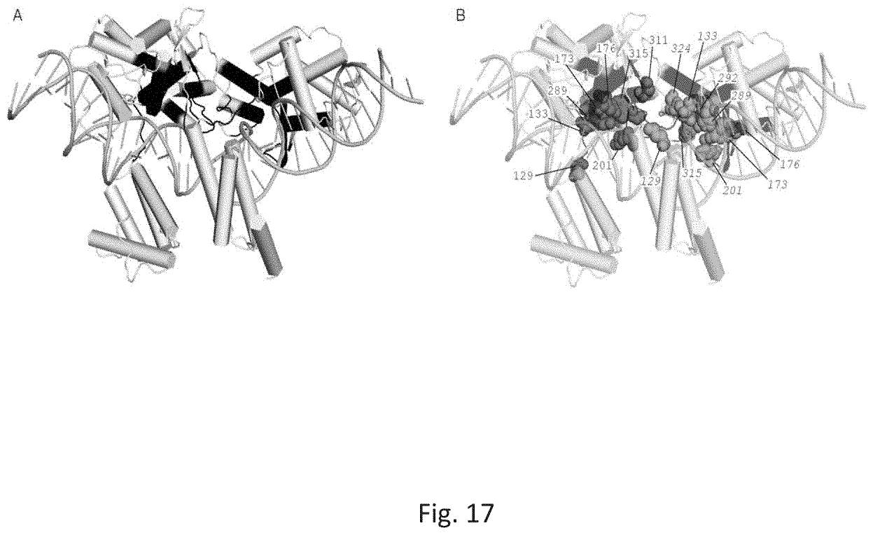

To generate further obligate recombinases, additional single mutations in the catalytic region (i.e. at amino acid positions 129-136, 163-181, 199-211, 289-301, 310-316 and 321-324 of SEQ ID NO: 1, cf. FIG. 17A, dark regions) of the recombination synapse of the Cre recombinase (Guo et al. 1997, Lee et al. 2002, Gibb et al. 2010, Meinke et al. 2016) were introduced as outlined in Table 6 below. A more detailed overview of the mutated positions in Cre is provided in FIG. 17B. The experiments show that an inactivating mutation in the catalytic region of a first recombinase monomer can be rescued by a different inactiviating mutation in the catalytic region of a second recombinase monomer. In the present example, Cre recombinase mutants of SEQ ID NO: 1 with a single amino acid substitution in the catalytic region were generated and expressed on their loxP target site to evaluate if the introduced mutation renders the monomer inactive. In a second step, two different inactive monomers were co-expressed on their loxP target site to evaluate if the two inactive monomers in combination can rescue the recombinase activity. Recombination activity of the recombinases on loxP was measured by a plasmid-based assay as previously described (6, 9, 10). in the experiments, the recombined pEVO is smaller in size compared to the non-recombined pEVO. The bigger fragment (^{˜}5 kb) shows non-recombined substrate, while the smaller fragment (^{˜}4.3 kb) shows recombined substrate. These two different fragments can either be distinguished by gel electrophoresis or by sequencing. The recombination efficiencies (indicated as dimer and monomer acitivty, respectively) were calculated based on the ratio of recombined and non-recombined substrate using the following formula: Recombination activity (%)=100×(recombined substrate/(recombined+non-recombined substrates)).

TABLE 6

| | | | 1st | 2nd |
|---|---|---|---|---|
| | | Median | monomer | monomer |
| 1st | 2nd | dimer | median | median |
| Monomer | Monomer | activity | activity | activity |
| E129R | K201N | 35% | 0% | 0% |
| E129R | K201R | 26% | 0% | 0% |
| E129R | Q311R | 93% | 0% | 3% |
| E129R | W315N | 30% | 0% | 0% |
| Q133H | R292A | 48% | 0% | 0% |
| Q133H | Q311R | 96% | 0% | 3% |
| Q133H | W315N | 33% | 0% | 0% |
| R173A | Q311R | 93% | 0% | 3% |
| R173C | E176H | 65% | 0% | 3% |
| R173C | E176M | 27% | 0% | 7% |
| R173C | Q311R | 91% | 0% | 3% |
| R173D | E176H | 29% | 0% | 3% |
| R173D | E176V | 53% | 0% | 3% |
| R173D | Q311R | 89% | 0% | 3% |
| R173E | E176H | 32% | 0% | 3% |
| R173E | Q311R | 80% | 0% | 3% |
| R173F | E176I | 25% | 0% | 0% |
| R173F | E176M | 38% | 0% | 7% |
| R173F | E176V | 82% | 0% | 3% |
| R173F | E176W | 30% | 0% | 0% |
| R173F | E176Y | 40% | 0% | 0% |
| R173F | K201Q | 39% | 0% | 0% |
| R173F | Q311R | 84% | 0% | 3% |
| R173G | K201Y | 95% | 0% | 0% |
| R173G | Q311R | 95% | 0% | 3% |
| R173G | Y324T | 32% | 0% | 0% |
| R173I | E176H | 42% | 0% | 3% |
| R173I | E176I | 40% | 0% | 0% |
| R173I | E176V | 44% | 0% | 3% |
| R173I | Q311R | 93% | 0% | 3% |
| R173K | Q311R | 84% | 1% | 3% |
| R173L | E176H | 66% | 0% | 3% |
| R173L | K201F | 41% | 0% | 0% |
| R173L | Q311R | 94% | 0% | 3% |
| R173L | W315M | 63% | 0% | 0% |
| R173L | Y324N | 88% | 0% | 0% |
| R173M | E176H | 75% | 0% | 3% |
| R173M | E176M | 39% | 0% | 7% |
| R173M | E176V | 75% | 0% | 3% |
| R173M | E176Y | 27% | 0% | 0% |
| R173M | Q311R | 96% | 0% | 3% |
| R173N | R292W | 44% | 0% | 0% |
| R173N | Q311R | 96% | 0% | 3% |
| R173N | W315I | 38% | 0% | 0% |
| R173N | Y324M | 51% | 0% | 0% |
| R173P | Q311R | 94% | 0% | 3% |
| R173P | Y324T | 47% | 0% | 0% |
| R173Q | E176M | 32% | 0% | 7% |
| R173Q | Q311R | 92% | 0% | 3% |
| R173S | E176V | 49% | 0% | 3% |
| R173S | Q311R | 97% | 0% | 3% |
| R173S | Y324E | 59% | 0% | 0% |
| R173T | E176H | 56% | 0% | 3% |
| R173T | E176I | 72% | 0% | 0% |
| R173T | E176V | 65% | 0% | 3% |
| R173T | K201W | 91% | 0% | 0% |
| R173T | Q311R | 92% | 0% | 3% |
| R173V | E176V | 53% | 0% | 3% |
| R173V | Q311R | 92% | 0% | 3% |
| R173W | E176H | 57% | 0% | 3% |
| R173W | E176Y | 30% | 0% | 0% |
| R173W | Q311R | 95% | 0% | 3% |
| R173Y | E176V | 35% | 0% | 3% |
| R173Y | Q311R | 92% | 0% | 3% |
| E176H | K201A | 33% | 3% | 0% |
| E176H | R292A | 55% | 3% | 0% |
| E176H | R292E | 38% | 3% | 0% |
| E176H | R292S | 68% | 3% | 0% |
| E176H | Q311R | 51% | 3% | 3% |
| E176I | R292M | 43% | 0% | 0% |
| E176I | R292S | 40% | 0% | 0% |
| E176I | Q311R | 36% | 0% | 3% |
| E176I | Y324F | 67% | 0% | 0% |
| E176I | Y324W | 50% | 0% | 0% |
| E176L | Q311R | 40% | 0% | 3% |
| E176M | K201R | 35% | 7% | 0% |
| E176M | Q311R | 82% | 7% | 3% |
| E176V | R292I | 33% | 3% | 0% |
| E176V | R292N | 31% | 3% | 0% |
| E176V | R292S | 28% | 3% | 0% |
| E176V | R292T | 45% | 3% | 0% |
| E176W | K201Q | 28% | 0% | 0% |
| E176W | Q311R | 90% | 0% | 3% |
| E176Y | K201R | 39% | 0% | 0% |
| E176Y | Q311R | 79% | 0% | 3% |
| K201A | K201R | 64% | 0% | 0% |
| K201A | Q311R | 95% | 0% | 3% |
| K201C | R292Q | 52% | 0% | 0% |
| K201C | Q311R | 93% | 0% | 3% |
| K201D | K201P | 70% | 0% | 0% |
| K201F | K201I | 72% | 0% | 0% |
| K201F | K201R | 59% | 0% | 0% |
| K201F | Q311R | 94% | 0% | 3% |
| K201F | W315R | 29% | 0% | 0% |
| K201F | W315T | 41% | 0% | 0% |
| K201F | Y324C | 98% | 0% | 0% |
| K201F | Y324F | 54% | 0% | 0% |
| K201G | Q311R | 90% | 0% | 3% |
| K201H | Q311R | 94% | 0% | 3% |
| K201I | Q311R | 86% | 0% | 3% |
| K201L | Q311R | 92% | 0% | 3% |
| K201L | Y324T | 25% | 0% | 0% |
| K201M | K201R | 76% | 0% | 0% |
| K201M | Q311R | 93% | 0% | 3% |
| K201N | Q311R | 95% | 0% | 3% |
| K201N | Y324A | 26% | 0% | 0% |
| K201P | Q311R | 95% | 0% | 3% |
| K201P | Y324L | 59% | 0% | 0% |
| K201Q | R292G | 58% | 0% | 0% |
| K201Q | Q311R | 94% | 0% | 3% |
| K201Q | W315I | 30% | 0% | 0% |
| K201R | Q311R | 98% | 0% | 3% |
| K201S | Q311R | 95% | 0% | 3% |
| K201T | Q311R | 94% | 0% | 3% |
| K201V | Q311R | 84% | 0% | 3% |
| K201V | W315M | 64% | 0% | 0% |
| K201V | Y324F | 48% | 0% | 0% |
| K201W | Q311R | 95% | 0% | 3% |
| K201W | W315M | 49% | 0% | 0% |
| K201W | Y324T | 68% | 0% | 0% |
| K201Y | Q311R | 90% | 0% | 3% |
| K201Y | Y324V | 26% | 0% | 0% |
| H289D | Q311R | 97% | 0% | 3% |
| H289E | Q311R | 94% | 0% | 3% |
| H289E | W315L | 33% | 0% | 0% |
| H289I | Q311R | 75% | 2% | 3% |
| H289K | Q311R | 95% | 0% | 3% |
| H289K | W315M | 74% | 0% | 0% |
| H289R | Q311R | 79% | 0% | 3% |
| H289W | Q311R | 95% | 0% | 3% |
| H289W | W315C | 87% | 0% | 0% |
| R292A | Q311R | 96% | 0% | 3% |
| R292C | Q311R | 93% | 0% | 3% |
| R292E | Q311R | 50% | 0% | 3% |
| R292E | Y324I | 70% | 0% | 0% |
| R292F | Q311R | 42% | 0% | 3% |
| R292G | Q311R | 97% | 0% | 3% |
| R292H | Q311R | 88% | 0% | 3% |
| R292I | Q311R | 97% | 0% | 3% |
| R292L | Q311R | 93% | 0% | 3% |
| R292M | Q311R | 96% | 0% | 3% |
| R292M | W315I | 42% | 0% | 0% |
| R292N | Q311R | 95% | 0% | 3% |
| R292P | Q311R | 76% | 0% | 3% |
| R292Q | Q311R | 70% | 0% | 3% |
| R292S | Q311R | 97% | 0% | 3% |
| R292T | Q311R | 94% | 0% | 3% |
| R292T | Y324K | 55% | 0% | 0% |
| R292V | Q311R | 95% | 0% | 3% |
| R292W | Q311R | 88% | 0% | 3% |
| R292Y | Q311R | 96% | 0% | 3% |
| R292Y | Y324M | 90% | 0% | 0% |
| Q311R | W315C | 84% | 3% | 0% |
| Q311R | W315I | 92% | 3% | 0% |
| Q311R | W315L | 68% | 3% | 0% |
| Q311R | W315M | 95% | 3% | 0% |
| Q311R | W315N | 64% | 3% | 0% |
| Q311R | W315Q | 49% | 3% | 0% |
| Q311R | W315S | 37% | 3% | 0% |
| Q311R | W315T | 66% | 3% | 0% |
| Q311R | W315V | 91% | 3% | 0% |
| Q311R | Y324A | 93% | 3% | 0% |
| Q311R | Y324C | 93% | 3% | 0% |
| Q311R | Y324F | 95% | 3% | 0% |
| Q311R | Y324H | 76% | 3% | 0% |
| Q311R | Y324I | 96% | 3% | 0% |
| Q311R | Y324K | 85% | 3% | 0% |
| Q311R | Y324L | 95% | 3% | 0% |
| Q311R | Y324M | 96% | 3% | 0% |
| Q311R | Y324N | 81% | 3% | 0% |
| Q311R | Y324Q | 72% | 3% | 0% |
| Q311R | Y324R | 61% | 3% | 0% |
| Q311R | Y324S | 81% | 3% | 0% |
| Q311R | Y324T | 85% | 3% | 0% |
| Q311R | Y324V | 90% | 3% | 0% |
| Q311R | Y324W | 95% | 3% | 0% |
| W315C | Y324N | 38% | 0% | 0% |
| W315C | Y324R | 31% | 0% | 0% |
| W315C | Y324T | 36% | 0% | 0% |
| W315E | Y324S | 35% | 0% | 0% |
| W315G | Y324M | 35% | 0% | 0% |
| W315G | Y324V | 37% | 0% | 0% |
| W315K | Y324K | 99% | 0% | 0% |
| W315M | Y324L | 71% | 0% | 0% |
| W315Q | Y324V | 97% | 0% | 0% |
| W315L | W315T | 50% | 0% | 0% |

Results Achieved with the Examples of the Invention

1. Monomer-Monomer Interface Mutations Reduce Recombination Activity

To form obligate hetero-specific Cre-type SSR complexes for asymmetric substrates, previous work has focused on redesigning the protein-protein interface of the interacting monomers. In particular, Zhang and coworkers have engineered an obligate Cre and a Cre-variant heterotetramer to recombine the artificial asymmetric loxM7/loxP target sequence (16). Key positions for the interface redesign were selected from predicted mutations that would form an alternative interaction surface between the wild-type Cre molecule and the Cre variant. To investigate whether the same interface mutations can be adopted for other heterospecific Cre-type SSRs, D7-variants were generated by mutating the two subunits to potentially form an obligate D7 recombinase. Therefore, the D7L variant (D7L^{A3}) was generated by mutating positions K25R, D29R, R32E, D33L, Q35R, E123L and R337E, whereas the D7R variant (D7RB²) harbored the mutations E69D, R72K, L76E and E308R.

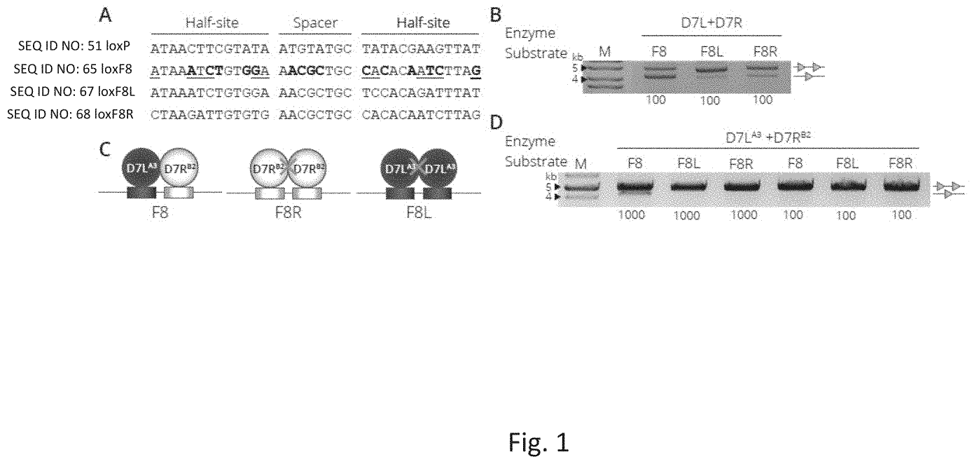

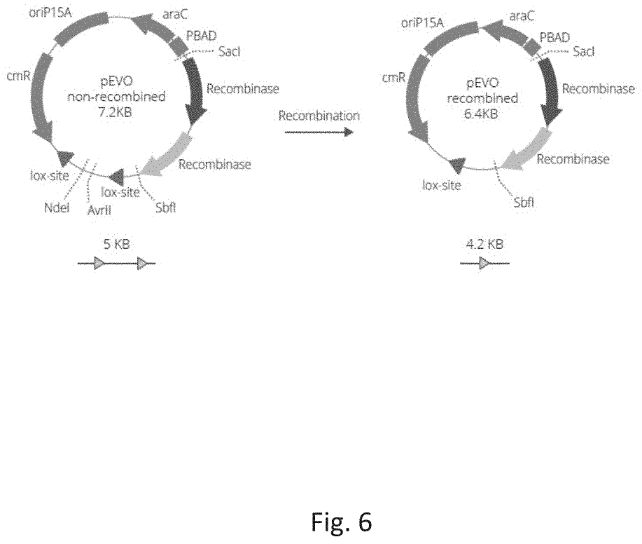

In order to compare the recombination efficiency before and after the applied mutations, first, the non-mutated D7L and D7R were co-expressed from a vector carrying either the loxF8, loxF8L or loxF8R target site as excision substrates (FIG. 1A and FIG. 6). As expected, co-expression of both D7L and D7R monomers resulted in efficient recombination on the asymmetric loxF8 target site (FIG. 1B). Recombination was also observed for both the symmetric loxF8L and loxF8R target sites (FIG. 1B), presumably due to functional homotetramer formation of their corresponding evolved recombinase. Introduction of the interface mutations (D7L^{A3} and D7R^{B2}) should prevent the homotetramer assembly, and thereby block recombination on the symmetric loxF8L and loxF8R sites, while the different subunits should still be able to form active heterotetramers on the asymmetric loxF8 sequence (FIG. 1C). Indeed, co-expression of D7LA³ and D7RB² did not lead to detectable recombination from vectors carrying the symmetric loxF8L or loxF8R sequences (FIG. 1D), indicating that these mutations prevented the formation of active homotetramers.

However, co-expression of D7L^{A3} and D7R^{B2} on the asymmetric loxF8 site lead to no observable activity compared to the activity of the original D7L+D7R complex at the same induction concentration of L-Arabinose (100 μg/mL). To determine if the co-expressed D7L^{A3} and D7R^{B2} recombinases were capable of forming an active complex, induction was increased to 1000 μg/mL L-Arabinose resulting in very low activity on the loxF8 site, proving that the complex is functional, just very inefficient (FIG. 1D). Hence, the applied mutations worked in principle, but also influenced the recombination activity of the D7 heterotetramer. Therefore, it was sought to recover the activity by searching for combinations of amino acid changes more suitable for an obligate D7 system.

2. Substrate-Linked Directed Evolution to Evolve Obligate D7 Recombinases with High Activity

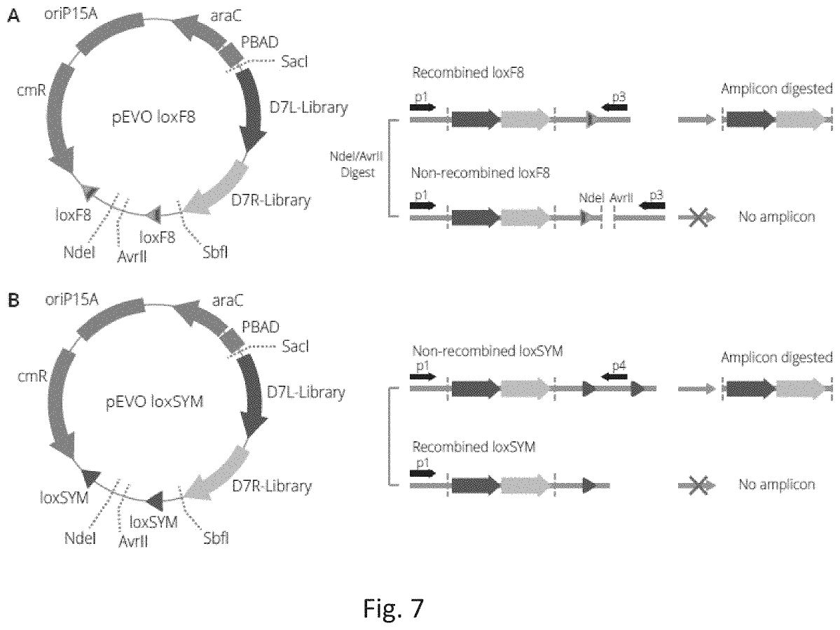

To search for beneficial residue changes, the generation of two libraries of D7L and D7R recombinase variants was started around the previously described (16) residue positions (A3-K25, D29R, R32E, D33L, Q35R, E123L and R337E and B2-E69D, R72K, L76E and E308R) involved in the protein-protein interface. The libraries were applied to the well-established substrate-linked directed evolution (SLiDE) procedure (6, 18). To keep the library to a practical screening size, diversity was directed to a subset of residue positions, A3 positions K25, D29, R32 and D33 and B2 positions E69, R72 and L76, located along the largest monomer-monomer interface. At each of these positions, mutations were limited to a subset of amino acids previously predicted for the interface redesign (D, E, H, I, K, M, N, Q, S, A, G, L, P, T, V, R) (16). The two D7L and D7R starting libraries were cloned into the corresponding vectors to begin iterative positive selection for activity on the asymmetric site (loxF8) and negative selection on the symmetric sites (loxF8L and loxF8R) through a modified version of SLiDE (FIG. 2A and FIG. 7A). By evolving the two libraries together instead of in parallel, functional pairs with the desired activity profile could be selected. After 26 cycles of SLiDE, we detected a marked increase in recombination activity for the asymmetric site and negligible recombination on the symmetric sites (FIG. 2B), indicating that heterodimeric pairs of recombinases with desirable features had evolved.

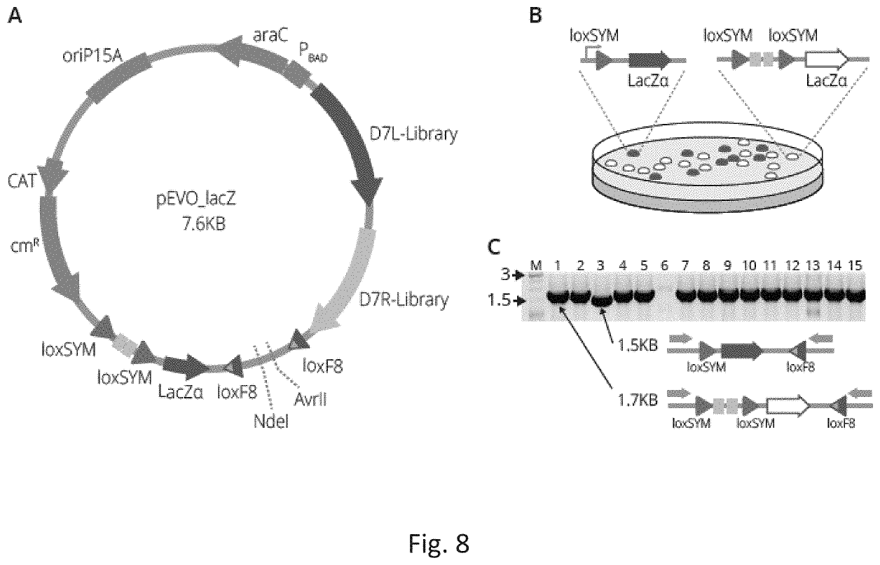

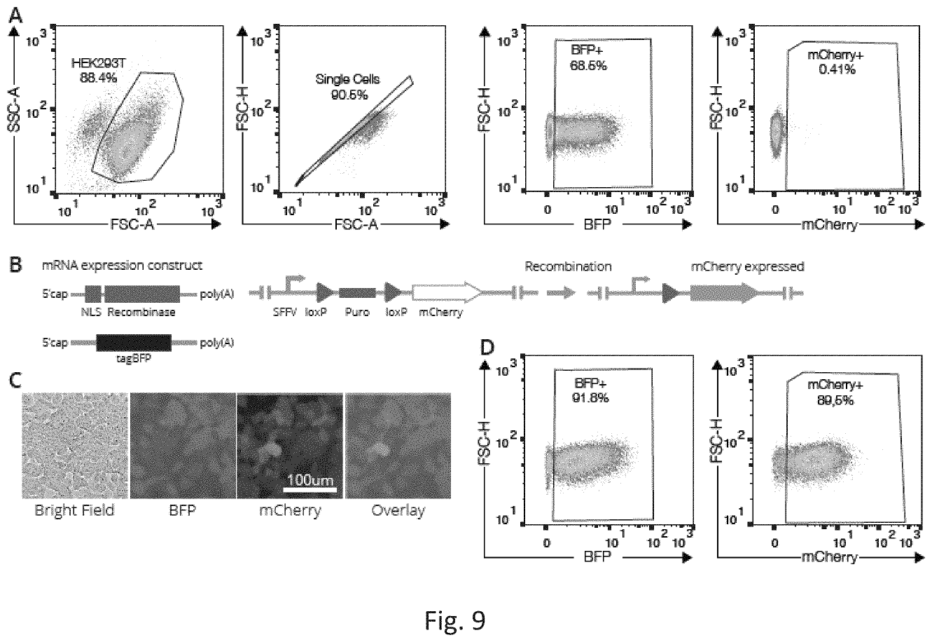

To eliminate any carry over of inactive recombinase variants that have leaked through selection, single variant pairs were assessed by using a blue-white colony screen. The selection plasmid (pEVO-LacZa) allowed for simultaneous identification of variants that did not recombine the symmetric sites while showing high activity on the asymmetric loxF8 site (FIG. 8A-C). 75 white colonies were selected and the encoded recombinase pairs were sequenced. Surprisingly, none of the hypermutated amino acid positions (69, 72, 76 and 308) were found to be enriched in the evolved D7R recombinases. In contrast, the glutamine at position 311 was changed in 41% (31 of 75 variants) of the sequenced D7R recombinases, where 87% (27 of 31) of the mutants displayed an arginine at this position. The results indicate that the initial targeted residues did not lead to the desired outcome and instead a single substitution that had occurred randomly during evolution was preferred (i.e. Q311R), presumably preventing recombination on the symmetric loxF8R site while maintaining activity when co-expressed with D7L variants on the asymmetric loxF8 sequence.

Sequencing the D7L-derived clones uncovered five positions that were mutated in more than 20% of the sequenced clones (positions 25, 29, 20, 282 and 305, FIG. 2C). Positions 25 and 29 most likely derived from the initial targeted residues involved in protein-protein interactions, while positions 201, 282 and 305 were not targeted in the start library, implying that they arose through random mutations and selection during directed evolution.

The most surprising result was the frequently mutated position 201, which was found to be changed in 48% (36 out of 75) of the clones, with all clones harboring an arginine at this position rather than a lysine (FIG. 2C). What makes this alteration so interesting is that lysine 201 is highly conserved throughout the tyrosine SSR family (19) and the residue has been described to be essential for the catalytic activity of Cre (20-22). K201 has been observed to function as an active residue within the Cre complex to facilitate DNA cleavage during recombination (20, 23). Hence, recombinases with alterations at position 201 would not be expected to exhibit any recombinase activity. On the basis of this research, it was aimed to further explore if mutating the catalytic K201 residue in D7L would inactivate the SSR when expressed as a monomer and if that was the case, if activity could be rescued by the presence of the paired Q311R mutation on the D7R monomer to perform recombination.

To determine the effects on recombination of the mutations applied to each monomer, their ability to recombine their original targets when expressed in isolation as monomers was first evaluated. Sole expression of D7L^{K201R} or D7R^{Q311R} on the symmetric loxF8L or loxF8R sites, respectively, did not lead to detectable recombination events, demonstrating that these mutations inactivate the enzymes when expressed in isolation (FIG. 2D). Furthermore, D7L^{K201R} and D7R^{Q311}R were inactive on the asymmetric loxF8 site when expressed in isolation (FIG. 2D). In sharp contrast, when D7L^{K201R} and D7R^{Q311R} were co-expressed, efficient recombination on the asymmetric loxF8 was observed (FIG. 2E), whereas no recombination was detectable on the symmetric loxF8L and loxF8R sites (FIG. 2E). Importantly, loxF8 sites were recombined at a similar rate compared to the original wild-type D7 clone (FIG. 2E), indicating that the two mutations did not compromise activity of the overall recombinase complex. Therefore, with just one mutation applied to each recombinase monomer, an obligate SSR complex with comparable activity to the wild-type D7 SSR was obtained.

3. D7^{K201R}+D7R^{Q311R} Support Obligate Recombination in Mammalian Cells

Because the D7 recombinase is targeted for applications within the human genome, the next step was to examine the activity of the obligate D7^{K201R}+D7R^{Q311R} complex in human cells. To allow straight-forward quantification, recombination efficiency in a HEK293T reporter cell line (10) was measured. The reporter cell line was co-transfected with mRNA carrying the recombinases along with an mRNA coding for tagBFP to monitor transfection efficiencies (FIG. 3A). Transfection of the obligate D7^{K201R}+D7R^{Q311R} molecules revealed a recombination efficiency of 81% compared to 79% recombination efficiency of wild-type D7 (FIG. 3B, C), implying that the introduction of the obligate mutations did not compromise recombinase activity in human cells. Importantly, transfecting each mutant monomer subunit in isolation yielded no significant loxF8 target site recombination (FIG. 3B, C).

The D7 recombinase was originally generated to correct the genomic int1h inversion frequently found in hemophilia A patients (24). The enzyme recognizes two loxF8 sequences that are found on the human X-chromosome at a distance of 140 kb from one another. The first site is present in intron 1 of the factor VIII gene and the second site is located 130 kb upstream of the factor VIII transcription start site (10, 25, 26). D7 has been shown to efficiently invert the displaced exon 1 sequence flanked by the loxF8 target sites upon expression in human cells (10). To confirm the ability of the D7^{K201R}+D7R^{Q311R} variants to act on these sites at the endogenous locus, genomic

DNA from HEK293T cells transfected with D7L^{K201R} and D7R^{Q311}R mRNAs were extracted and ran a PCR based assay designed to detect the inversion of exon 1 (FIG. 3D). Indeed, expression of D7^{K201R}+D7R^{Q311R} led to inversion of the genomic fragment (FIG. 3E), demonstrating that the obligate mutations do not interfere with the activity of the recombinase to recombine this disease-causing inversion.

To evaluate if the D7^{K201R}+D7R^{Q311R} heterodimer improved target site specificity, its activity on four predicted human off-target sites was analzed (FIG. 3F), employing a plasmid-based activity assay (Supplemental FIG. 1). Consistent with previous data (10), the wild-type D7 recombinase displayed no detectible activity on 3 of the 4 sequences, but showed activity on the HG2L off-target site (FIG. 3G). In comparison, the obligate D7^{K201R}+D7R^{Q311R} complex showed no detectable activity on all four of the predicted off-target sites (FIG. 3G), demonstrating its improved applied properties. Together, these results show that the D7^{K201R}+D7R^{Q311R} heterodimer promotes target site specificity while maintaining comparable recombination efficiency for the loxF8 target site in mammalian cells.

4. K201R and Q311R mutations render Cre, Vika and Dre recombinases obligate

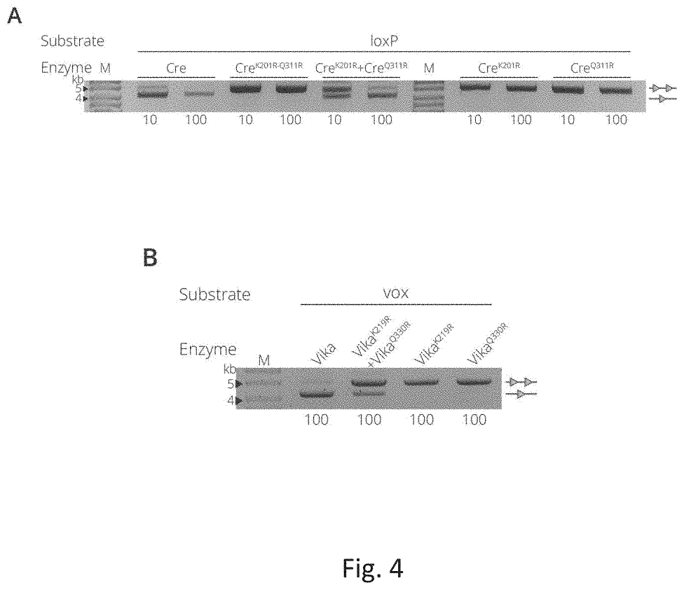

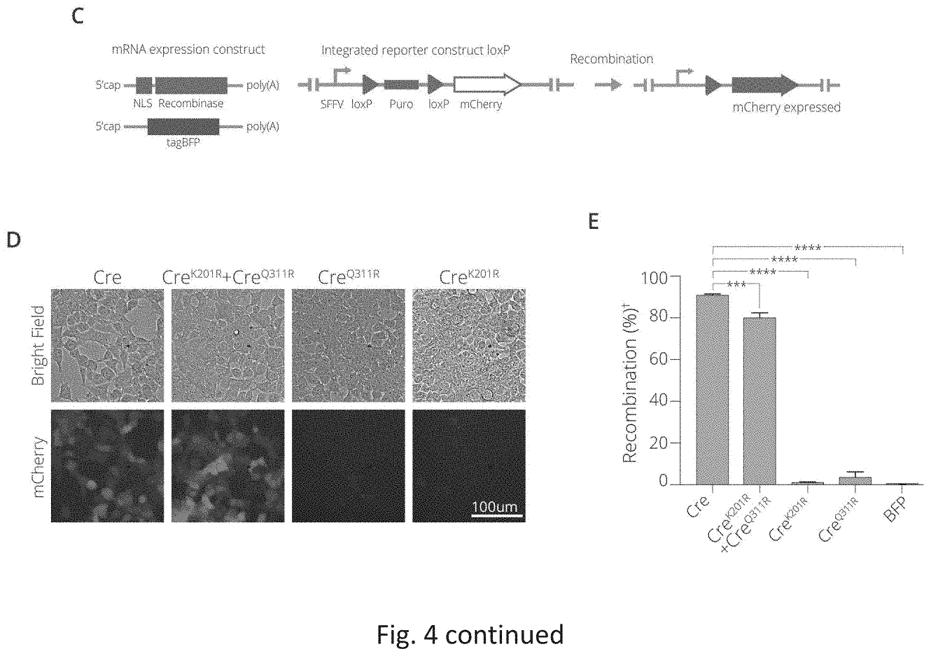

To explore a more general applicability and obtain insights into the molecular mechanism of the identified obligate SSR system, the phenotype of the corresponding mutations in two naturally occurring homotetrameric SSR complexes was investigated, namely Cre and Vika (27). To test the system, Cre^{K201R} and Cre^{Q311R w}ere generated. The obligate mutations were also incorporated into Vika at positions 219 and 330, according to the conserved sequences seen in the sequence alignment (27) forming two mutant monomers, namely Vika^{K219R} and Vika^{Q330R}. Activity was analyzed for both on an excision substrate in E. coli. When Cre^{K201R} or Cre^{Q311R} were expressed in isolation, no recombination was observed on the loxP targets (FIG. 4A). To test whether both mutations introduced into the same monomer results in recombination, CreK201R+Q311R were generated. Analysis of the Cre double mutant on an excision substrate in E. coli showed no observable recombination of the loxP target sequence indicating that the mutations have to be present on different monomers to allow for the formation of active SSR complexes (FIG. 4A). A similar activity profile (although with some loss of activity) was seen when Vika^{K219R} and Vika^{Q330R} were co-expressed on the vox target sequence, whereas no recombination was observed when the mutated monomers were expressed in isolation (FIG. 4B).

Next it was evaluated if the obligate Cre system could function efficiently in mammalian cells and maintain the recombination profile seen in bacteria. A HEK293T red fluorescent reporter cell line was transfected with SSR mRNAs to evaluate recombination activity (FIG. 4C). When the Cre mutants were expressed alone, negligible recombination activity was detected (FIG. 4D, 4E), signifying that Cre^{K201R} and Cre^{Q311R} expressed in isolation are inactive. In sharp contrast, co-expressed of Cre^{K201R} and Cre^{Q311R} yielded recombination efficiencies comparable to that of wild-type Cre on loxP (FIG. 4D, 4E). Together, these results demonstrate that the targeted mutations are not only applicable to the heterotetrametric D7/loxF8 complex, but can also be applied to obtain obligate systems of wild type SSRs found in nature.

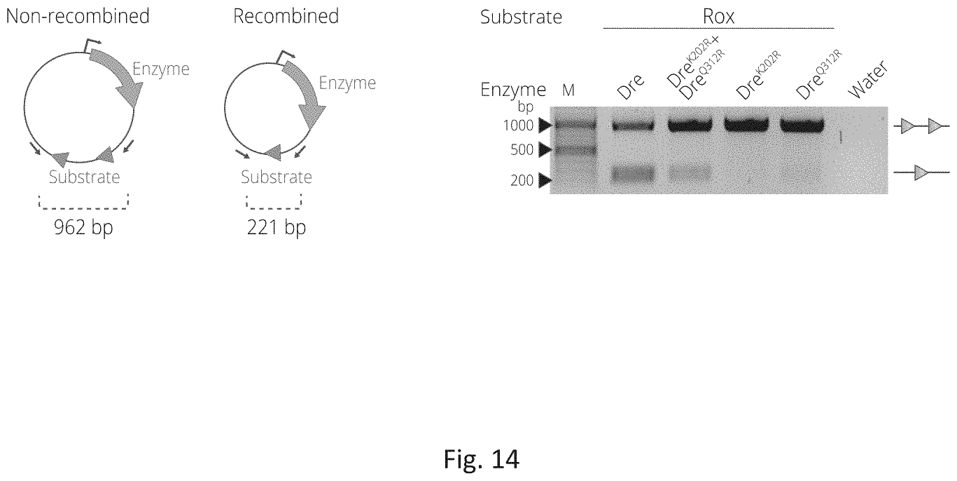

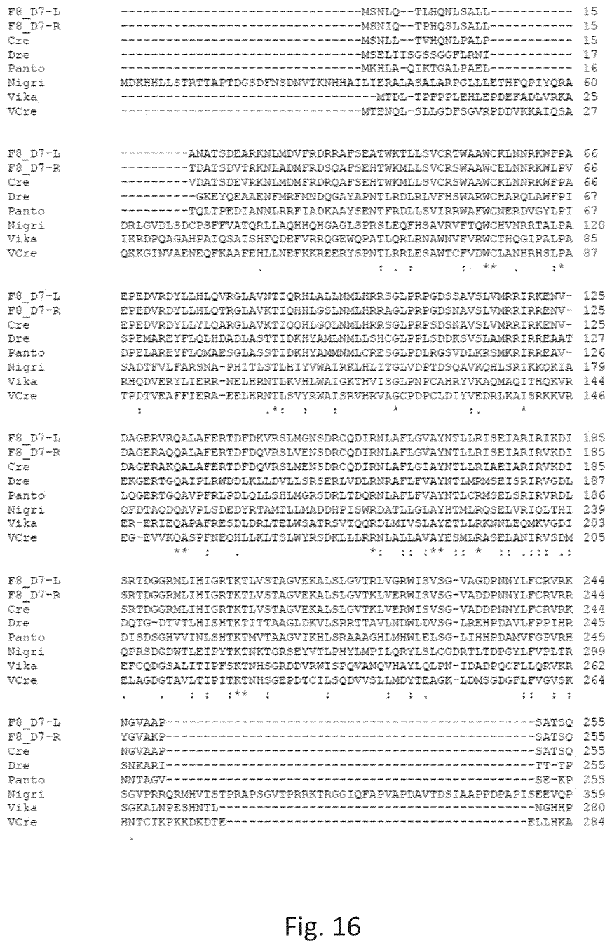

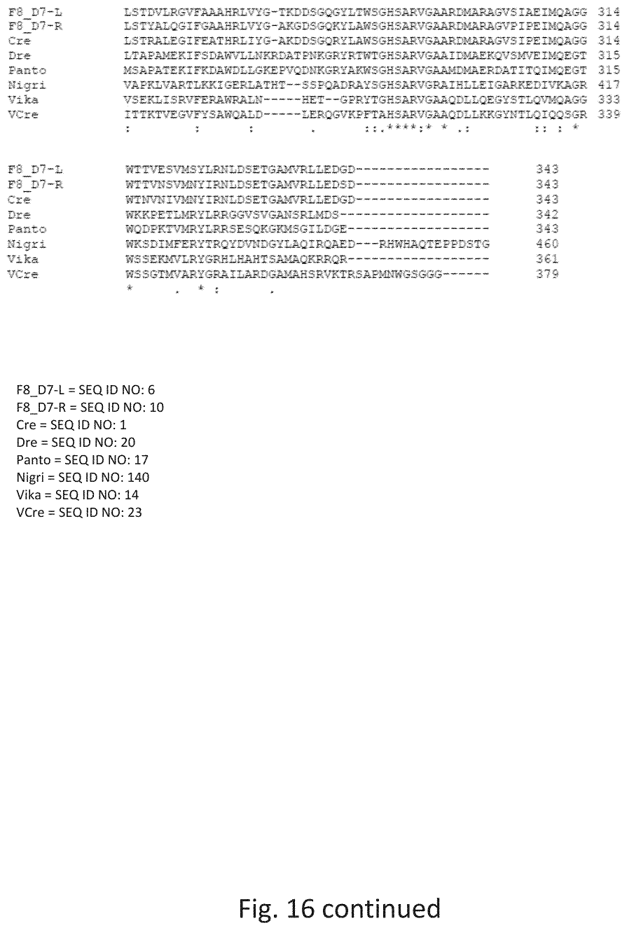

Further applicability of the obligate SSR system was tested in the naturally occurring Dre/rox complex. The obligate mutations were incorporated into Dre at positions 202 and 312 according to the conserved sequences seen in the alignment to Cre (FIG. 16) forming the DreK202R+DreQ312R/rox complex. To test the functionality of the mutant complex in E. coli, a sensitive PCR-based detection method was used due to the low activity of wild-type Dre (FIG. 14) (primers 23 and 24 from table 3). Amplification over the substrate confirms recombination of rox when both DreK202R and DreQ312R are present and minimal or complete loss of recombination when the obligate Dre mutants are expressed in isolation. . The results of the PCR-based assay are shown in FIG. 14.

5. Molecular Modeling Supports Mechanism for Catalysis Driving Obligate Heterotetramer Formation

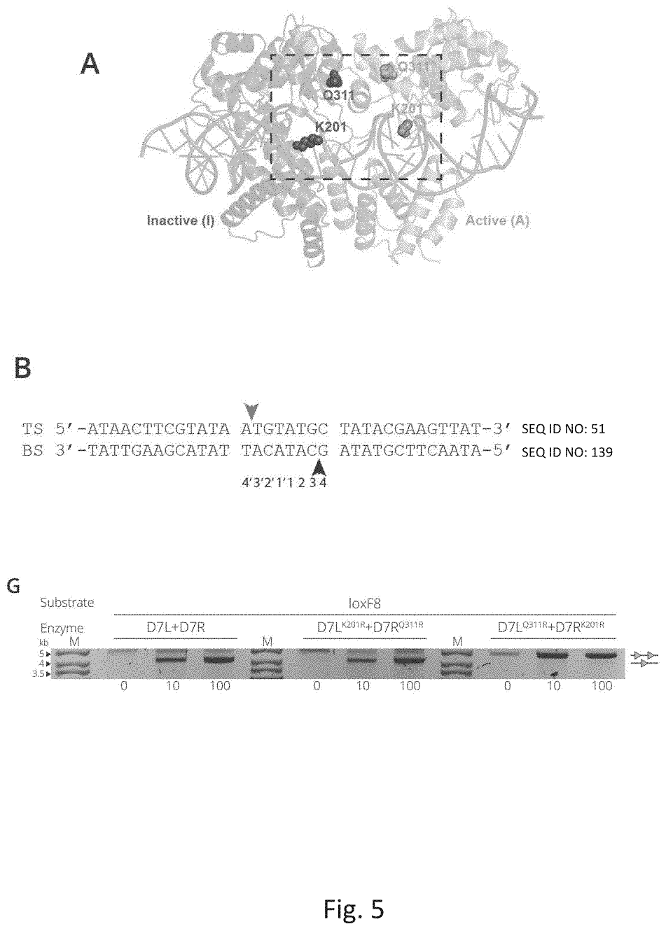

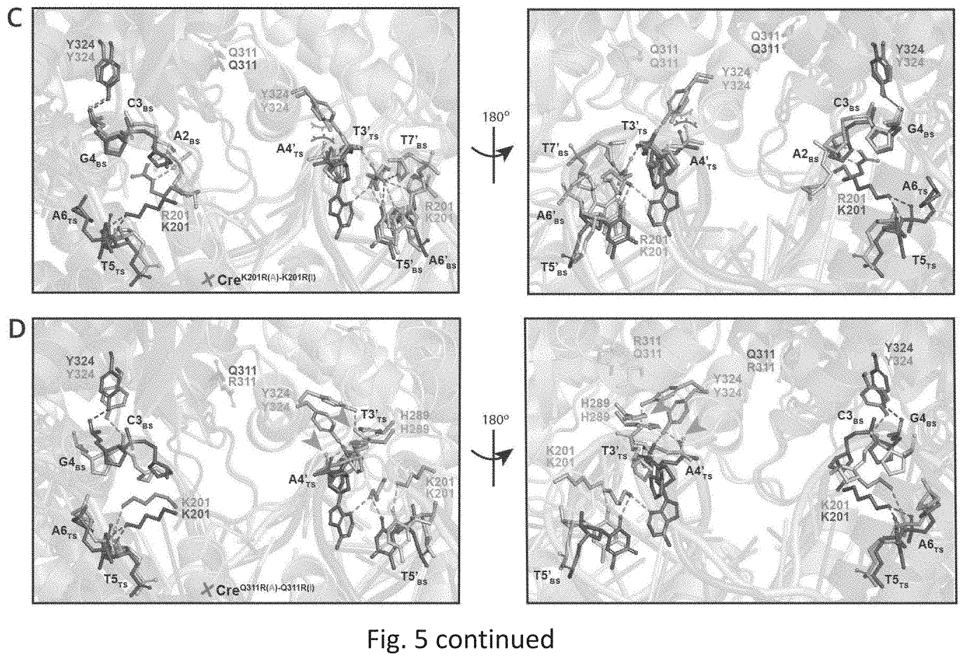

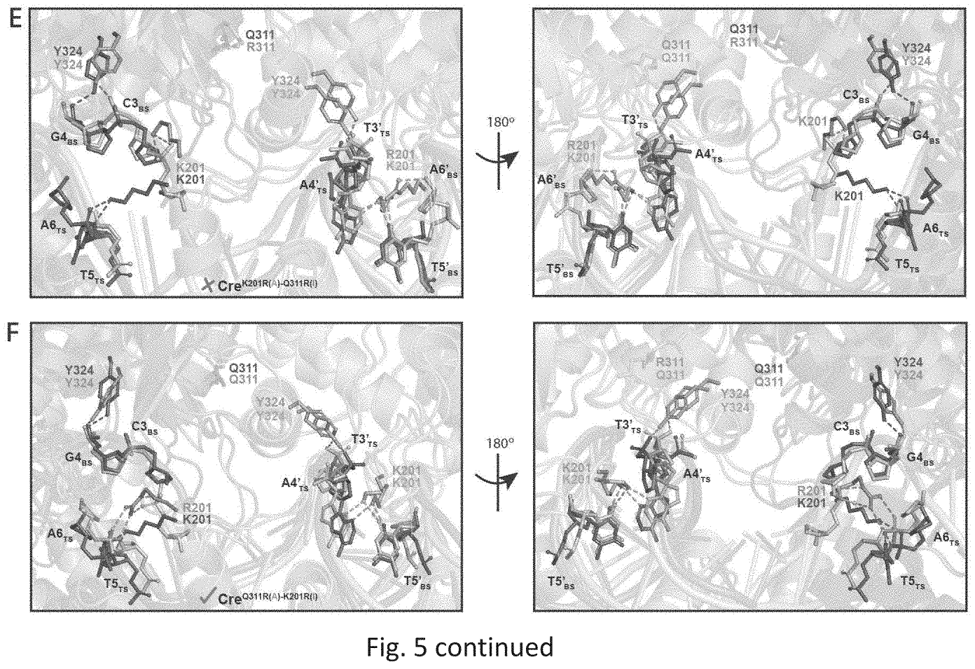

To obtain a more mechanistic understanding of the obligate mutations, molecular models of CreK^{201R} and Cre^{Q311R} bound to the loxP target site were created, based on the Cre co-crystal structure with the highest resolution (PDB: 3C29), followed by extensive molecular dynamic simulation analyses. These analyses revealed why the single mutants, Cre^{K201R} and Cre^{Q311R}, are inactive (FIG. 5). For the Cre^{K201R} mutant, the arginine 201 had shifted dramatically in the inactive subunit to now interact with the DNA backbone at base A2 of the bottom strand, instead of interacting with base T5 on the top strand, thereby compromising recombination proficiency. Furthermore, the catalytic tyrosine 324 of the active subunit was displaced from base T3′ on the top strand and now formed a hydrogen bond with base A4′, explaining why Cre^{K201R} is inactive.

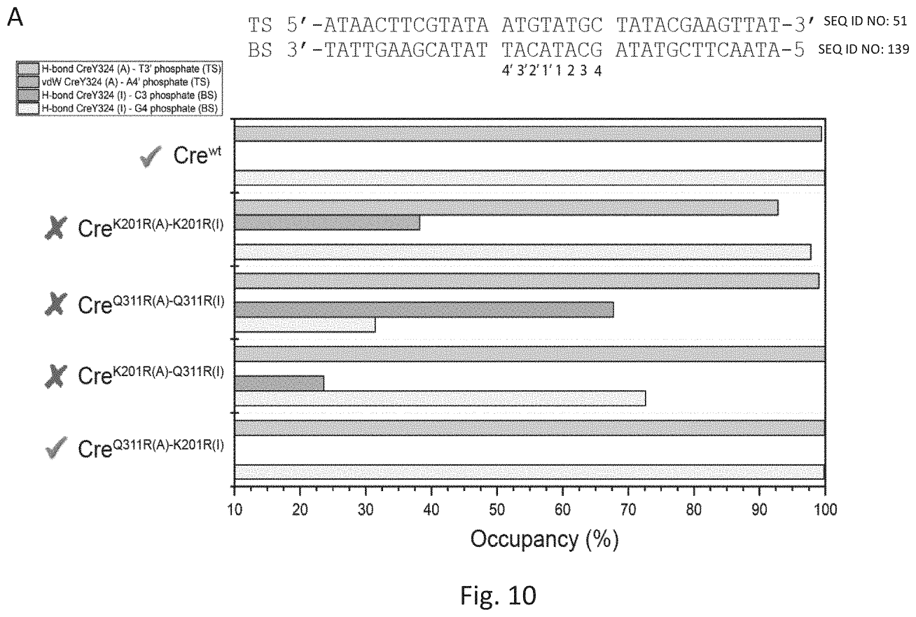

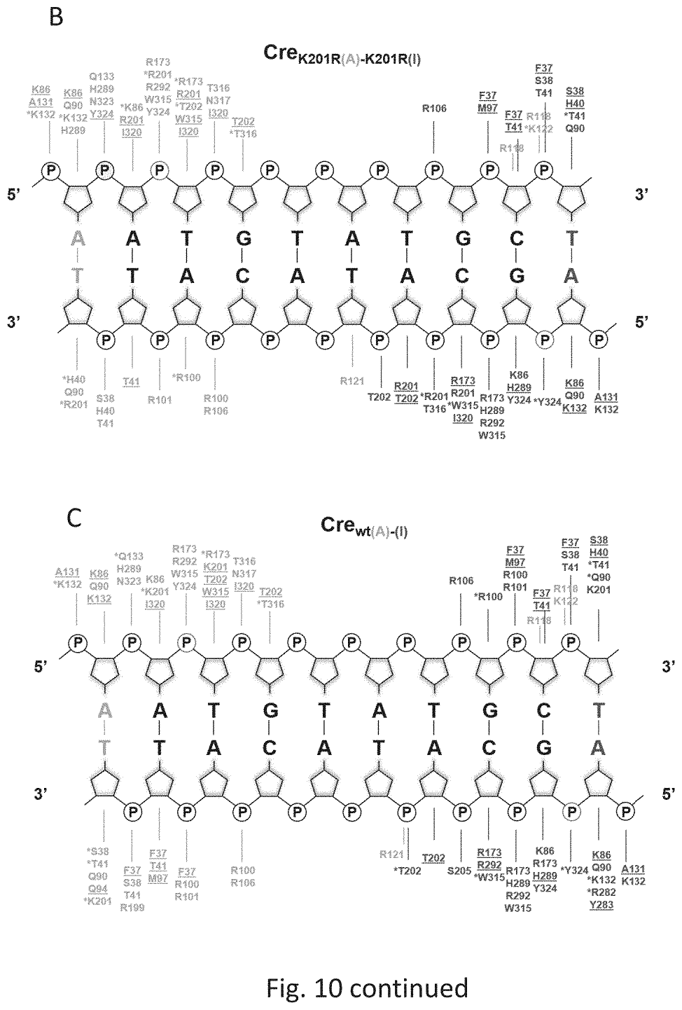

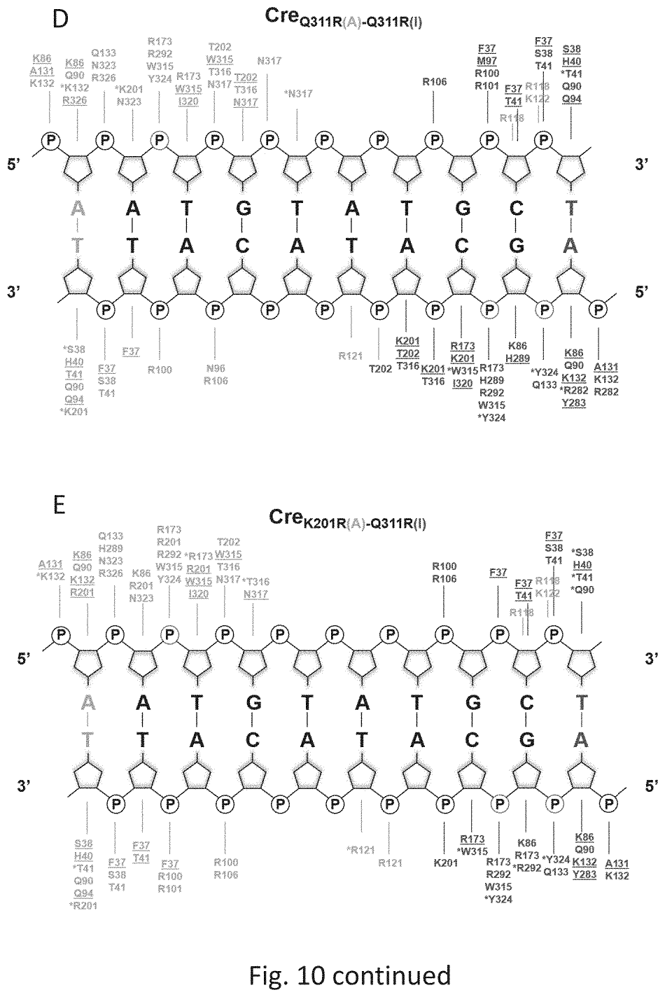

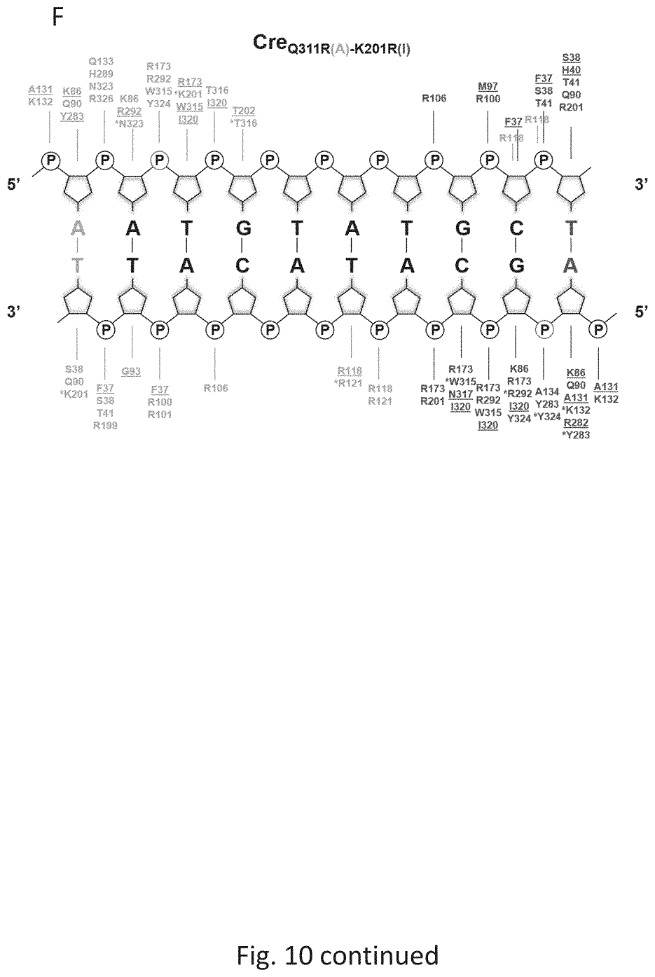

For the Cre^{Q311R} mutant, it was observed that the catalytic tyrosine 324 was displaced in both the active and the inactive subunit, while K201 lost important interactions with the DNA backbone. Furthermore, another residue known to play an important role in recombination catalysis (H289) was altered (FIG. 10D).

Next, the model where the K201R mutation was introduced into the active subunit was analyzed, while the Q311R mutations was placed into the inactive subunit (FIG. 10E). Surprisingly, this configuration resulted in a model incompatible with active recombination. Tyrosine 324 was displaced in the inactive subunit to now interact with base C3 on the bottom strand, while K201 had moved dramatically to also interact with base C3 on the bottom strand. Hence, this configuration does not constitute an active recombinase.

Lastly, the model where the K201R mutation was introduced into the inactive subunit was analyzed, while the Q311R mutations was placed into the active subunit. This configuration was in agreement with an active enzyme, where all catalytic residues were positioned to allow recombination.

6. Single Point Mutations in the Catalytic Region of Recombination Synapse Leads to Obligate Phenotype

The present inventors further show that an inactivating mutation in the in the catalytic region of a monomer can be rescued by an inactivating mutation in the in the catalytic region of another monomer. Specifically, single Cre monomer mutants (see Table 4) were inactive on the loxP target site when expressed in isolation (see FIG. 18, grey bars). However, the activity on loxP was rescued when two different inactive monomers were co-expressed on loxP (see FIG. 18, black bars). The principle that catalytic impaired monomers can form an obligate heterocomplex was shown for 186 combinations (Table 6). Moreover, as the catalytic regions are conserved in the family of T-SSRs (see e.g. the alignment in FIG. 19), this principle can be applied for other DNA recombinases and specifically to other DNA recombinases, e.g. T-SSRs such as Vika, Dre and Panto recombinases.

Discussion of the Results of the Working Examples

By altering the DNA-specificity of Cre through engineering and directed evolution, distinct SSR variants can be generated that together recombine asymmetric target sequences as heterotetramers (6, 8-10). The generation of such heterotetrameric SSR systems substantially broadens the potential sequences that can be targeted within genomes. However, possible combinations of subunits could lead to active SSR byproducts capable of catalyzing off-target recombination. Previously, prevention of homotetramer formation was achieved through structure-guided redesign of several residues implicated in the protein-protein interaction interface between the different recombinase monomers (16). Hence, this approach to generate obligate SSR systems is limited to enzymes with available crystal structures and is therefore not easily adaptable to engineered or distantly related recombinases. The present inventors show that obligate SSR systems can also be generated by mutating amino acid residues in the catalytic region. Importantly, this novel way of generating obligate SSRs only required the alteration of one single conserved residue within each distinct SSR monomer. This simplified approach can potentially be applied to many engineered or natural SSRs, without prior structural knowledge of the enzymes.

In summary, the invention provides a simplistic approach to reduce off-target recombination and improved specificity of engineered and wild-type SSRs. In particular, the enhanced specificity of the D7L^{K201R} and D7RQ³¹¹R system at off-target sites was demonstrated. The provided data further supports the general concept that catalytically inactive monomers can be rescued when co-expressed with another catalytically inactive monomer. Importantly, this novel way of generating obligate SSRs only requires alteration of one residue within the catalytic region of each distinct SSR monomer. This simplified approach can be applied to many engineered or natural DNA recombinases without prior structural knowledge of the enzymes.

### REFERENCES

1. Duyne, G. D. V. (2015) Cre Recombinase. Microbial Spectr, 3, 119-138.
2. Meinke, G., Bohm, A., Hauber, J., Pisabarro, M. T. and Buchholz, F. (2016) Cre Recombinase and Other Tyrosine Recombinases. Chemical reviews, 116: 12785-12820.
3. Anastassiadis, K., Schnütgen, F., Melchner, H. von and Stewart, A. F. (2013) Chapter Nine Gene Targeting and Site-Specific Recombination in Mouse ES Cells. Methods Enzymol, 533, 133-155.
4. Monetti, C., Nishino, K., Biechele, S., Zhang, P., Baba, T., Woltjen, K. and Nagy, A. (2011) PhiC31 integrase facilitates genetic approaches combining multiple recombinases. Methods, 53, 380-385.
5. Saraf-Levy, T., Santoro, S. W., Volpin, H., Kushnirsky, T., Eyal, Y., Schultz, P. G., Gidoni, D. and Carmi, N. (2006) Site-specific recombination of asymmetric lox sites mediated by a heterotetrameric Cre recombinase complex. Bioorgan Med Chem, 14, 3081-3089.
6, Buchholz, F. and Stewart, A. F. (2001) Alteration of Cre recombinase site specificity by substrate-linked protein evolution. Nat Biotechnol, 19, 1047-4052.
7. Sarkar, I., Hauber, I., Hauber, J., and Buchholz, F. (2007) HIV-1. Proviral DNA Excision Using an Evolved Recombinase. Science, 316, 1912-1915.
8. Karpinski, J., Hauber, I., Chemnitz, J., Schäfer, C., Paszkowski-Rogacz, M., Chakraborty, D., Beschorner, N., Hofmann-Sieber, H., Lange, U. C., Grundhoff, A., et a (2016) Directed evolution of a recombinase that excises the provirus of most HIV-1 primary isolates with high specificity. Nat Biotechnol, 34, 401-409.
9. Lansing, F., Paszkowski-Rogacz, M., Schmitt, L. T., Schneider, P. M., Romanos, T. R., Sonntag, J. and Buchholz, F. (2020) A heterodimer of evolved designer-recombinases precisely excises a human genomic DNA locus. Nucleic Acids Res, 48, 472-485.
10. Lansing, F., Mukhametzyanova, L, Rojo-Romanos, T., Iwasawa, K., Kimura, M., Paszkowski-Rogacz, M., Karpinski, J., Grass, T., Sonntag, J., Schneider, P. M., et al. Correction of a Factor VIII genomic inversion with designer-recombinases. Nat Commun 13, 422 (2022)
11. Hauber, I., Hofmann-Sieber, H., Chemnitz, J., Dubrau, D., Chusainow, J., Stucka, R., Hartjen, P., Schambach, A., Ziegler, P., Hackmann, K., et al. (2013) Highly Significant Antiviral Activity of HIV-1 LTR-Specific Tre-Recombinase in Humanized Mice. Plos Pathog, 9, e1003587.
12. Bolusani, S., Ma, C.-H., Paek, A., Konieczka, J. H., Jayaram, M. and Voziyanov, Y. (2006) Evolution of variants of yeast site-specific recombinase Flp that utilize native genomic sequences as recombination target sites. Nucleic Acids Res, 34, 5259-5269.
13. Soni, A., Augsburg, M., Buchholz, F. and Pisabarro, M. T, (2020) Nearest-neighbor amino acids of specificity-determining residues influence the activity of engineered Cre-type recombinases. Sci Rep-uk, 10, 13985.
14. Abi-Ghanem, J., Chusainow, J., Karimova, M., Spiegel, C., Hofmann-Sieber, H., Hauber, J., Buchholz, F. and Pisabarro, M. T. (2013) Engineering of a target site-specific recombinase by a combined evolution- and structure-guided approach. Nucleic Acids Res, 41, 2394-2403.
15. Shah, R., Li, F., Voziyanova, E. and Voziyanov, Y. (2015) Target-specific variants of Flp recombinase mediate genome engineering reactions in mammalian cells. Febs J, 282, 3323-3333.
16. Zhang, C., Myers, C. A., Qi, Z., Mitra, R. D., Corbo, J. C. and Havranek, J. J. (2015) Redesign of the monomer-monomer interface of Cre recombinase yields an obligate heterotetrameric complex. Nucleic Acids Res, 43, 9076-9085.
17. Chen, Y.-J., Nielsen, A. A. K., Brophy, J. A. N., Clancy, K., Peterson, T. and Voigt, C. A. (2013) Characterization of 582 natural and synthetic terminators and quantification of their design constraints. Nat Methods, 10, 659-664.
18. Herman, A. and Tawfik, D. S. (2007) Incorporating Synthetic Oligonucleotides via Gene Reassembly (ISOR): a versatile tool for generating targeted libraries. Protein Eng Des Se!, 20, 219-226.
19. Esposito, D. and Scocca, J. J, (1997) The integrase family of tyrosine recombinases: evolution of a conserved active site domain. Nucleic Acids Res, 25, 3605-3614.
20. Gibb, B., Gupta, K., Ghosh, K., Sharp, R., Chen, J. and Duyne, G. D. V. (2010) Requirements for catalysis in the Cre recombinase active site. Nucleic Acids Res, 38, 5817-5832.
21. Martin, S. S., Chu, V. C, and Baldwin, E. (2003) Modulation of the Active Complex Assembly and Turnover Rate by Protein-DNA Interactions in Cre-LoxP Recombination , †, ‡. Biochemistry-us, 42, 6814--6826.
22. Luo, J., Liu, O., Morihiro, K. and Deiters, A. (2016) Small-molecule control of protein function through Staudinger reduction. Nat Chem, 8, 1027-1034.
23. Abi-Ghanem, J., Samsonov, S. A, and Pisabarro, M. T. (2015) Insights into the preferential order of strand exchange in the Cre/loxP recombinase system: impact of the DNA spacer flanking sequence and flexibility. J Comput Aid Mol Des, 29, 271-282,
24. Park, C.-Y., Kim, D. H., Sung, J. J., Bae, S., Kim, D.-W. and Kim, J.-S. (2015) Functional Correction of Large Factor VIII Gene Chromosomal Inversions in Hemophilia A Patient-Derived iPSCs Using CRISPR-Cas9. Cell Stern Cell, 17, 213-220.
25. Lannoy, N. and Hermans, C. (2016) Principles of genetic variations and molecular diseases: applications in hemophilia A. Crit Rev Oncol Hemat, 104, 1-8.
26. Oldenburg, J., Pezeshkpoor, B. and Pavlova, A. (2014) Historical Review on Genetic Analysis in Hemophilia A*. Seminars Thrombosis Hemostasis, 40, 895-902.
27. Karimova, M., Abi-Ghanem, J., Berger, N., Surendranath, V., Pisabarro, M. T. and Buchholz, F. (2013) Vika/vox, a novel efficient and specific Cre/loxP-like site-specific recombination system. Nucleic Acids Res, 41, e37-e37.
28. Chandras, C., Zouberakis, M., Salimova, E., Smedley, D., Rosenthal, N. and Aidinis, V. (2012) CreZOO—the European virtual repository of Cre and other targeted conditional driver strains. Database, 2012, bas029.
29. Murray, S. A., Smedley, D., Simpson, E. M. and Rosenthal, N. (2012) Beyond knockouts: cre resources for conditional mutagenesis. Mamm Genome, 23, 587-599.
30. He, L., Li, Y., Li, Y., Pu, W., Huang, X., Tian, X., Wang, Y., Zhang, H., Liu, Q., Zhang, L., et al. (2017) Enhancing the precision of genetic lineage tracing using dual recombinases. Nat Med, 23, 1488-4498.
31, Lapique, N. and Benenson, Y. (2014) Digital switching in a biosensor circuit via programmable timing of gene availability. Nat Chem Biol, 10, 1020-4027.
32. Yamanishi, M. and Matsuyama, T. (2012) A Modified Cr& lox Genetic Switch To Dynamically Control Metabolic Flow in Saccharomyces cerevisiae. Acs Synth Biol, 1, 172-180.
33. Petyuk, V., McDermott, J., Cook, M. and Sauer, B. (2004) Functional Mapping of Cre Recornbinase by Pentapeptide Insertional Mutagenesis. J Biol Chem, 279, 37040-37048.
34. Carroll, D. (2014). Genome engineering with targetable nucleases. Annual Review of Biochemistry, 83: 409-439.
35. Wang, M., Glass, Z. A., Xu, Q. (2017). Non-viral delivery of genome-editing nucleases for gene therapy. Gene Therapy, 24: 144-150.
36. Tebas, P., Stein, D., Tang, W., Frank, I., Wang, S. Q., Lee, G., Spratt, S. K., Surosky, R. T., Giedlin, M. A., Nichol, G., Holmes, M. C., Gregory, P. D., Ando, D. G., Kalos, M., Collman, R. G., Binder-Scholl, G., Plesa, G., Hwang, W. T., Levine, B. L., June, C. H. (2014). Gene editing of CCR5 in autologous CD4 T cells of persons infected with HIV. The New England Journal of Medicine, 370: 901-910.
37. Qasim, W., Amrolia, P. J., Samarasinghe, S., Ghorashian, S., Zhan, H., Stafford, S., Butler, K., Ahsan, G., Gilmour, K., Adams, S., Pinner, D., Chiesa, R., Chatters, S., Swift, S., Goulden, N., Peggs, K., Thrasher, A. J., Veys, P., & Pule, M. (2015). First Clinical Application of Talen Engineered Universal CAR19 T Cells in B-ALL. Blood, 126: 2046.
38. Cyranoski, D. (2016). CRISPR gene-editing tested in a person for the first time. Nature, 539: 479.
39. Cox, D. B., Platt, R. J., Zhang, F. (2015). Therapeutic Genome Editing: Prospects and Challenges. Nature Medicine 21: 121-131.
40. Kosicki, M., Tomberg, K., Bradley, A. (2018). Repair of double-strand breaks induced by CRISPR—Cas9 leads to large deletions and complex rearrangements. Nature Biotechnology, 36: 765-771.

1. A genetically engineered DNA recombining enzyme comprising a complex of at least a first DNA recombinase enzyme and at least a second DNA recombinase enzyme,
wherein said first DNA recombinase enzyme and said second DNA recombinase enzyme specifically recognize a first half-site and a second half-site of an upstream target site and/or a downstream target site of a DNA recombinase,
wherein said first DNA recombinase enzyme and said second DNA recombinase enzyme each comprises a single amino acid substitution in their catalytic region,
wherein said first DNA recombinase enzyme and said second DNA recombinase enzyme carrying said single amino acid substitution when in isolation do not show the catalytic activity of a DNA recombinase, and
wherein said first DNA recombinase enzyme and said second DNA recombinase enzyme carrying said single amino acid substitution when forming a complex show the catalytic activity of a DNA recombinase.

2. The DNA recombining enzyme according to claim 1, wherein the at least one first recombinase and the at least one second recombinase are of the same type, optionally wherein the at least one first recombinase and the at least one second recombinase are both Cre-, Dre-, VCre-, SCre-, Vika-, lambda-Int-, Flp-, R-, Kw-, Kd-, B2-, B3-, Nigri- or Panto-recombinases.

3. (canceled)

4. The DNA recombining enzyme according to claim 1, wherein the DNA recombining enzyme is a complex of recombinases in form of a heterotetramer.

5. The DNA recombining enzyme according to claim 1, wherein the single amino acid substitution in the at least one first and in the at least one second recombinase enzyme is at a position of a conserved amino acid in the catalytic region.

6. The DNA recombining enzyme according to claim 1, wherein:
(i) the first recombinase enzyme comprises a Cre recombinase and the second recombinase enzyme comprises a Cre recombinase, wherein each Cre recombinase comprises a single amino acid substitution within an amino acid region selected from the group consisting of SEQ ID NO: 109 to SEQ ID NO: 114;
(ii) the first recombinase enzyme comprises a Vika recombinase and the second recombinase enzyme comprises a Vika recombinase, wherein each Vika recombinase comprises a single amino acid substitution within an amino acid region selected from the group consisting of SEQ ID NO: 115 to SEQ ID NO: 120;
(iii) the first recombinase enzyme comprises a Dre recombinase and the second recombinase enzyme comprises a Dre recombinase, wherein each Dre recombinase comprises a single amino acid substitution within an amino acid region selected from the group consisting of SEQ ID NO: 121 to SEQ ID NO: 126; or
(iv) the first recombinase enzyme comprises a Panto recombinase and the second recombinase enzyme comprises a Panto recombinase, wherein each Panto recombinase comprises a single amino acid substitution within an amino acid region selected from the group consisting of SEQ ID NO: 127 to SEQ ID NO: 132.

7. The DNA recombining enzyme according to any one of the preceding claims claim 1, wherein the single amino acid substitution in the at least one first and in the at least one second recombinase enzyme is selected from the group consisting of: E129R, Q133H, R173A, R173C, R173D, R173E, R173F, R173G, R173I, R173K, R173L, R173M, R173N, R173P, R173Q, R173S, R173T, R173V, R173W, R173Y, E176H, E176I, E176L, E176M, E176V, E176W, E176Y, K201A, K201C, K201C, K201D, K201F, K201G, K201H, K201I, K201L, K201M, K201N, K201P, K201Q, K201R, K201S, K201T, K201V, K201W, K201Y, H289D, H289E, H289I, H289K, H289R, H289W, R292A, R292C, R292E, R292F, R292G, R292H, R292I, R292L, R292M, R292N, R292P, R292Q, R292S, R292T, R292V, R292W, R292Y, Q311R, W315C, W315E, W315G, W315I, W315K, W315L, W315M, W315N, W315Q, W315R, W315S, W315T, W315V, Y324A, Y324C, Y324E, Y324F, Y324H, Y324I, Y324K, Y324L, Y324M, Y324N, Y324Q, Y324R, Y324S, Y324T, Y324V, and Y324W of SEQ ID NO: 1, or in a corresponding position of another recombinase, optionally wherein the corresponding position of another recombinase is relative to a corresponding position of SEQ ID NO: 14, SEQ ID NO: 17 or SEQ ID NO: 20.

8. (canceled)

9. The DNA recombining enzyme according to claim 1, wherein the single amino acid substitution in the at least one first and in the at least one second recombinase enzyme is at a position selected from the group consisting of:
(i) E129, Q133, R173, E176, K201, H289, R292, Q311, W315, and Y324 of SEQ ID NO: 1;
(ii) E146, Q151, R191, N194, K219, H308, R311, Q330, W334, and Y343 of SEQ ID NO: 14;
(iii) E130, Q134, R174, E177, K202, H290, R293, Q312, W316, and Y325 of SEQ ID NO: 17;
(iv) E131, Q135, R175, E178, K202, H290, R293, Q312, W316, and Y325 of SEQ ID NO: 20; or
(v) at an amino acid position in another recombinase, wherein said amino acid position in the other recombinase corresponding to position E129, Q133, R173, E176, K201, H289, R292, Q311, W315, or Y324 of SEQ ID NO: 1.

10. A genetically engineered DNA recombining enzyme comprising a complex of at least a first DNA recombinase enzyme and at least a second DNA recombinase enzyme, wherein
(i) the first recombinase enzyme is a polypeptide having an amino acid sequence with at least 70%, 80%, or 90%, sequence identity with a sequence according to SEQ ID NO: 7 and comprises the single mutation K201R in its catalytic region; and wherein the second recombinase enzyme is a polypeptide having an amino acid sequence with at least 70%, 80%, or 90%, sequence identity with a sequence according to SEQ ID NO: 11 and comprises the single mutation Q311R in its catalytic region;
(ii) the first recombinase enzyme is a polypeptide which has an amino acid sequence having at least 70%, 80%, or 90%, sequence identity with a sequence according to SEQ ID NO: 7 and comprises the single mutation K201R in its catalytic region; and wherein the second recombinase enzyme is a polypeptide having an amino acid sequence with at least 70%, 80%, or 90%, sequence identity with a sequence according to SEQ ID NO: 12 and comprises the single mutation Q311K in its catalytic region.

11. A DNA recombining enzyme comprising:
(i) a first Cre recombinase comprising the single mutation K201R in the catalytic region and having an amino acid sequence which is at least 70%, 80%, or 90%, identical with a polypeptide having the sequence according to SEQ ID NO: 2; and a second Cre recombinase comprising the single mutation Q311R in the catalytic region and having an amino acid sequence which is at least 70%, 80%, or 90%, identical with a polypeptide having the sequence according to SEQ ID NO: 5;
(ii) a first Cre recombinase comprising the single mutation K201R in the catalytic region and having an amino acid sequence which is at least 70%, 80%, or 90%, identical with a polypeptide having the sequence according to SEQ ID NO: 2; and a second Cre recombinase comprising the single mutation Q311K in the catalytic region and having an amino acid sequence which is at least 70%, 80%, or 90%, identical with a polypeptide having the sequence according to SEQ ID NO: 4;
(iii) a first Vika recombinase comprising the single mutation K219R in the catalytic region and having an amino acid sequence which is at least 70%, 80%, or 90%, identical with a polypeptide having the sequence according to SEQ ID NO: 15; and a second Vika recombinase comprising the single mutation Q330R in the catalytic region and having an amino acid sequence which is at least 70%, 80%, or 90%, identical with a polypeptide having the sequence according to SEQ ID NO: 16;
(iv) a first Panto recombinase comprising the single mutation K202R in the catalytic region and having an amino acid sequence which is at least 70%, 80%, or 90%, identical with a polypeptide having the sequence according to SEQ ID NO: 18; and a second Panto recombinase comprising the single mutation Q312R in the catalytic region and having an amino acid sequence which is at least 70%, 80%, or 90%, identical with a polypeptide having the sequence according to SEQ ID NO: 19;
(v) a first Dre recombinase comprising the single mutation K202R in the catalytic region and having an amino acid sequence which is at least 70%, 80%, or 90%, identical with a polypeptide having the sequence according to SEQ ID NO: 21; and a second Dre recombinase comprising the single mutation Q312R in the catalytic region and having an amino acid sequence which is at least 70%, 80%, or 90%, identical with a polypeptide having the sequence according to SEQ ID NO: 22; or
(vi) a first Vcre recombinase comprising the single mutation K221R in the catalytic region and having an amino acid sequence which is at least 70%, 80%, or 90%, identical with a polypeptide having the sequence according to SEQ ID NO: 24; and a second Vcre recombinase comprising the single mutation Q336R in the catalytic region and having an amino acid sequence which is at least 70%, 80%, or 90%, identical with a polypeptide having the sequence according to SEQ ID NO: 25.

12. A nucleic acid molecule or a plurality of nucleic acid molecules each comprising or consisting of a nucleic acid sequence encoding the genetically engineered DNA recombining enzyme, or the at least one first DNA recombinase enzyme and/or the at least one second DNA recombinase enzyme, according to claim 1.

13. An expression vector comprising the nucleic acid molecule or the plurality of nucleic acid molecules according to claim 12 and one or more expression-controlling elements operably linked with said nucleic acid to drive expression thereof.

14. A host cell comprising the expression vector according to claim 13.

15. A pharmaceutical composition comprising the at least one first DNA recombinase enzyme and/or the at least one second DNA recombinase enzyme according to claim 1, and one or more therapeutically acceptable diluents or carriers.

16. A method for generating obligate DNA recombinases for genome editing, wherein said method comprises the steps of:
(i) providing a nucleic acid molecule encoding a first recombinase enzyme and a nucleic acid molecule encoding a second recombinase enzyme, wherein said first recombinase enzyme binds to a first half site of an asymmetric recombinase target site and said second recombinase enzyme binds to a second half site of an asymmetric recombinase target site, wherein said first recombinase enzyme and said second recombinase enzyme form a heterodimer which is capable of inducing a site-specific DNA recombination of a sequence of interest at said asymmetric recombinase target site in a DNA sequence, wherein said asymmetric recombinase target site comprises a first half site and a second half site of an upstream target site and/or a downstream target site of a DNA recombinase an upstream and a downstream half site which wherein said first half site and a second half site are not identical and which are not palindromic;
(ii) mutagenesis to create libraries of nucleic acid molecules encoding mutant first recombinase enzymes and of nucleic acid molecules encoding mutant second recombinase enzymes, wherein mutations are introduced in said first recombinase enzyme and said second recombinase enzyme;
(iii) creating expression vectors, by cloning the library of the nucleic acid molecules encoding a first mutant recombinase enzyme and the library of nucleic acid molecules encoding a second mutant recombinase enzyme into expression vectors, wherein said expression vectors carry a DNA sequence of interest, which is to be recombined;
(iv) transfecting a cell with the expression vectors of step iii) and expressing the libraries of said mutant first recombinase enzyme and said mutant second recombinase enzyme in the same cell resulting in the formation of recombinase heterodimers comprising a mutant first recombinase enzyme and a mutant second recombinase enzyme;
(v) performing positive selection screens for heterodimers obtained in step iv. that are capable to induce a site-specific DNA recombination of a sequence of interest at an asymmetric recombinase target site in a DNA;
(vi) performing negative selection screens for heterodimers obtained in step iv. or v. that are not capable to induce a site-specific DNA recombination of a sequence of interest at an off-target, preferably symmetric recombinase target site in a DNA;
(vii) selecting an obligate DNA recombinase which is capable of recombining a DNA sequence of interest at a recombinase target site in a DNA comprising a first half site and a second half site of an upstream target site and/or a downstream target site of a DNA recombinase, and which is not capable of recombining a DNA sequence of interest at an off-target, preferably symmetric recombinase target site in a DNA;
wherein in said obligate DNA recombinase obtained in step (vii), said first mutant recombinase enzyme and said second mutant recombinase enzyme each comprises at least one mutation in a catalytic site which renders said first recombinase enzyme and said second recombinase enzyme catalytically inactive when expressed in isolation.

17. A method for generating a complex of obligate DNA recombinase enzymes, said method comprising the steps of:
(i) introducing a single amino acid substitution into the catalytic region of a first DNA recombinase enzyme, wherein said single amino acid substitution renders the first DNA recombinase enzyme catalytically inactive;
(ii) introducing a single amino acid substitution into the catalytic region of a second DNA recombinase enzyme, wherein said single amino acid substitution renders the second DNA recombinase enzyme catalytically inactive;
(iii) co-expressing both the mutated first and the mutated second DNA recombinase enzymes in a host cell;
(iv) isolating the mutated first and the mutated second DNA recombinase enzymes from the host cell.

18. A DNA recombining enzyme obtained by the method of claim 16.

19. An in vitro method for inversion of a DNA sequence on genomic level in a cell, comprising the steps of:
(i) providing a nucleic acid molecule encoding a first recombinase enzyme of claim 1, wherein said first recombinase enzyme specifically recognizes a first half-site of a recombinase target site;
(ii) providing a nucleic acid molecule encoding a second recombinase enzyme of claim 1, wherein said second recombinase enzyme specifically recognizes a second half-site of a recombinase target site;
(iii) creating an expression vector by cloning the nucleic acid molecule encoding the first recombinase enzyme and the nucleic acid molecule encoding the second recombinase enzyme into an expression vector;
(iv) delivering said expression vector to a cell, which comprises a DNA sequence to be inverted;
(v) expressing the first recombinase enzyme and the second recombinase enzyme in said cell;
(vi) allowing formation of a DNA recombinase complex comprising the first recombinase enzyme and the second recombinase enzyme; and
(vii) allowing inversion of the DNA sequence to be inverted in said cell.

20. A method for treating or preventing a disease, comprising administering to a subject in need thereof the genetically engineered DNA recombining enzyme according to claim 1, optionally wherein the disease comprises hemophilia A, optionally wherein the hemophilia A is severe hemophilia A.

21. (canceled)

22. A method for recombination of a target DNA sequence in a cell, comprising introducing into the cell:
(a) a nucleic acid molecule encoding the first recombinase enzyme and a nucleic acid molecule encoding the second recombinase enzyme of claim 1; and/or
(b) the first recombinase enzyme and the second recombinase of claim 1,
thereby recombining the target DNA sequence in the cell, optionally wherein the nucleic acid molecule encoding the first recombinase enzyme and/or the nucleic acid molecule encoding the second recombinase enzyme is an mRNA or an expression vector.

23-24. (canceled)
